(19) United States
(12) Patent Application Publication
(10) Pub. No.: US 2004/0043953 A1
(43) Pub. Date: Mar. 4, 2004

(54) Genes of corynebacterium

(76) Inventors: Markus Pompejus, Freinsheim (DE); Burkhard Kroger, Limburgerhof (DE); Oskar Zelder, Speyer (DE); Hartwig Schroder, Nubloch (DE)

Correspondence Address: NIXON & VANDERHYE, PC, 1100 N GLEBE ROAD, 8TH FLOOR, ARLINGTON, VA, 22201-4714, US

(21) Appl. No.: 10/450,055

(22) PCT Filed: Dec. 22, 2000
(86) PCT No.: PCT/EP00/13143

(51) Int. Cl. C12Q 1/68; C07H 21/04; C07K 14/195; C12P 21/02; C12N 1/21

(52) U.S. Cl. 514/44; 536/23.7; 435/69.3; 435/252.3; 435/6; 435/320.1; 530/350

(57) ABSTRACT

Isolated nucleic acid molecules, designated MP nucleic acid molecules, which encode novel MP proteins from Corynebacterium glutamicum are described. The invention also provides antisense nucleic acid molecules, recombinant expression vectors containing MP nucleic acid molecules, and host cells into which the expression vectors have been introduced. The invention still further provides isolated MP proteins, mutated MP proteins, fusion proteins, antigenic peptides and methods for the improvement of production of a desired compound from C. glutamicum based on genetic engineering of MP genes in this organism.

Isolated nucleic acid molecules, designated MP nucleic acid molecules, which encode novel MP proteins from Corynebacterium glutamicum are described. The invention also provides antisense nucleic acid molecules, recombinant expression vectors containing MP nucleic acid molecules, and host cells into which the expression vectors have been introduced. The invention still further provides isolated MP proteins, mutated MP proteins, fusion proteins, antigenic peptides and methods for the improvement of production of a desired compound from C. glutamicum based on genetic engineering of MP genes in this organism.

Certain products and by-products of naturally-occurring metabolic processes in cells have utility in a wide array of industries, including the food, feed, cosmetics, and pharmaceutical industries. These molecules, collectively termed ‘fine chemicals’, include organic acids, both proteinogenic and non-proteinogenic amino acids, nucleotides and nucleosides, lipids and fatty acids, diols, carbohydrates, aromatic compounds, vitamins and cofactors, and enzymes. Their production is most conveniently performed through large-scale culture of bacteria developed to produce and secrete large quantities of a particular desired molecule. One particularly useful organism for this purpose is Corynebacterium glutamicum, a gram positive, nonpathogenic bacterium. Through strain selection, a number of mutant strains have been developed which produce an array of desirable compounds. However, selection of strains improved for the production of a particular molecule is a time-consuming and difficult process.

The invention provides novel bacterial nucleic acid molecules which have a variety of uses. These uses include the identification of microorganisms which can be used to produce fine chemicals, the modulation of fine chemical production in C. glutamicum or related bacteria, the typing or identification of C. glutamicum or related bacteria, as reference points for mapping the C. glutamicum genome, and as markers for transformation. These novel nucleic acid molecules encode proteins, referred to herein as metabolic pathway (MP) proteins.

C. glutamicum is a gram positive, aerobic bacterium which is commonly used in industry for the large-scale production of a variety of fine chemicals, and also for the degradation of hydrocarbons (such as in petroleum spills) and for the oxidation of terpenoids. The MP nucleic acid molecules of the invention, therefore, can be used to identify microorganisms which can be used to produce fine chemicals, e.g., by fermentation processes. Modulation of the expression of the MP nucleic acids of the invention, or modification of the sequence of the MP nucleic acid molecules of the invention, can be used to modulate the production of one or more fine chemicals from a microorganism (e.g., to improve the yield or production of one or more fine chemicals from a Corynebacterium or Brevibacterium species).

The MP nucleic acids of the invention may also be used to identify an organism as being Corynebacterium glutamicum or a close relative thereof, or to identify the presence of C. glutamicum or a relative thereof in a mixed population of microorganisms. The invention provides the nucleic acid sequences of a number of C. glutamicum genes; by probing the extracted genomic DNA of a culture of a unique or mixed population of microorganisms under stringent conditions with a probe spanning a region of a C. glutamicum gene which is unique to this organism, one can ascertain whether this organism is present. Although Corynebacterium glutamicum itself is nonpathogenic, it is related to species pathogenic in humans, such as Corynebacterium diphtheriae (the causative agent of diphtheria); the detection of such organisms is of significant clinical relevance.

The MP nucleic acid molecules of the invention may also serve as reference points for mapping of the C. glutamicum genome, or of genomes of related organisms. Similarly, these molecules, or variants or portions thereof, may serve as markers for genetically engineered Corynebacterium or Brevibacterium species.

The MP proteins encoded by the novel nucleic acid molecules of the invention are capable of, for example, performing an enzymatic step involved in the metabolism of certain fine chemicals, including amino acids, vitamins, cofactors, nutraceuticals, nucleotides, nucleosides, and trehalose. Given the availability of cloning vectors for use in Corynebacterium glutamicum, such as those disclosed in Sinskey et al., U.S. Pat. No. 4,649,119, and techniques for genetic manipulation of C. glutamicum and the related Brevibacterium species (e.g., lactofermentum) (Yoshihama et al, J. Bacteriol. 162: 591-597 (1985); Katsumata et al., J. Bacteriol. 159: 306-311 (1984); and Santamaria et al., J. Gen. Microbiol. 130: 2237-2246 (1984)), the nucleic acid molecules of the invention may be utilized in the genetic engineering of this organism to make it a better or more efficient producer of one or more fine chemicals.

This improved production or efficiency of production of a fine chemical may be due to a direct effect of manipulation of a gene of the invention, or it may be due to an indirect effect of such manipulation. Specifically, alterations in C. glutamicum metabolic pathways for amino acids, vitamins, cofactors, nucleotides, and trehalose may have a direct impact on the overall production of one or more of these desired compounds from this organism. For example, optimizing the activity of a trehalose or a lysine or a methionine biosynthetic pathway protein or decreasing the activity of a trehalose or a lysine or methionine degradative pathway protein may result in an increase in the yield or efficiency of production of trehalose or lysine or methionine from such an engineered organism. Alterations in the proteins involved in these metabolic pathways may also have an indirect impact on the production or efficiency of production of a desired fine chemical. For example, a reaction which is in competition for an intermediate necessary for the production of a desired molecule may be eliminated, or a pathway necessary for the production of a particular intermediate for a desired compound may be optimized. Further, modulations in the biosynthesis or degradation of, for example, an amino acid, a vitamin, or a nucleotide may increase the overall ability of the microorganism to rapidly grow and divide, thus increasing the number and/or production capacities of the microorganism in culture and thereby increasing the possible yield of the desired fine chemical.

The nucleic acid and protein molecules of the invention may be utilized to directly improve the production or efficiency of production of one or more desired fine chemicals from Corynebacterium glutamicum. Using recombinant genetic techniques well known in the art, one or more of the biosynthetic or degradative enzymes of the invention for amino acids, vitamins, cofactors, nutraceuticals, nucleotides, nucleosides, or trehalose may be manipulated such that its function is modulated. For example, a biosynthetic enzyme may be improved in efficiency, or its allosteric control region destroyed such that feedback inhibition of production of the compound is prevented. Similarly, a degradative enzyme may be deleted or modified by substitution, deletion, or addition such that its degradative activity is lessened for the desired compound without impairing the viability of the cell. In each case, the overall yield or rate of production of the desired fine chemical may be increased.

It is also possible that such alterations in the protein and nucleotide molecules of the invention may improve the production of other fine chemicals besides the amino acids, vitamins, cofactors, nutraceuticals, nucleotides, nucleosides, and trehalose through indirect mechanisms. Metabolism of any one compound is necessarily intertwined with other biosynthetic and degradative pathways within the cell, and necessary cofactors, intermediates, or substrates in one pathway are likely supplied or limited by another such pathway. Therefore, by modulating the activity of one or more of the proteins of the invention, the production or efficiency of activity of another fine chemical biosynthetic or degradative pathway may be impacted. For example, amino acids serve as the structural units of all proteins, yet may be present intracellularly in levels which are limiting for protein synthesis; therefore, by increasing the efficiency of production or the yields of one or more amino acids within the cell, proteins, such as biosynthetic or degradative proteins, may be more readily synthesized. Likewise, an alteration in a metabolic pathway enzyme such that a particular side reaction becomes more or less favored may result in the over- or under-production of one or more compounds which are utilized as intermediates or substrates for the production of a desired fine chemical.

This invention provides novel nucleic acid molecules which encode proteins, referred to herein as metabolic pathway proteins (MP), which are capable of, for example, performing an enzymatic step involved in the metabolism of molecules important for the normal functioning of cells, such as amino acids, vitamins, cofactors, nucleotides and nucleosides, or trehalose. Nucleic acid molecules encoding an MP protein are referred to herein as MP nucleic acid molecules. In a preferred embodiment, the MP protein performs an enzymatic step related to the metabolism of one or more of the following: amino acids, vitamins, cofactors, nutraceuticals, nucleotides, nucleosides, and trehalose. Examples of such proteins include those encoded by the genes set forth in Table 1.

TABLE 1
Genes in the Application

| Nucleic Acid | Amino Acid | Gene Function | |
|---|---|---|---|
| SEQ ID NO | SEQ ID NO | (identifier) | Function |
| 1 | 2 | metH | 5-Methyltetrahydrofolate-homocysteine |
| | | | methyltransferase (EC 2.1.1.13) |
| 3 | 4 | treS | Trehalose Synthase |

Accordingly, one aspect of the invention pertains to isolated nucleic acid molecules (e.g., cDNAs, DNAs, or RNAs) comprising a nucleotide sequence encoding an MP protein or biologically active portions thereof, as well as nucleic acid fragments suitable as primers or hybridization probes for the detection or amplification of MP-encoding nucleic acid (e.g., DNA or mRNA). In particularly preferred embodiments, the isolated nucleic acid molecule comprises one of the nucleotide sequences set forth as the odd-numbered SEQ ID NOs in the Sequence Listing (SEQ ID NO:1, SEQ ID NO:3), or the coding region or a complement thereof of one of these nucleotide sequences. In other particularly preferred embodiments, the isolated nucleic acid molecule of the invention comprises a nucleotide sequence which hybridizes to or is at least about 63%, preferably at least about 71%, more preferably at least about 75%, 80% or 90%, and even more preferably at least about 95%, 96%, 97%, 98%, 99% or more homologous to a nucleotide sequence which encodes a proteine sequence set forth as an even-numbered SEQ ID NO in the Sequence Listing (SEQ ID NO:2, SEQ ID NO:4), or a portion thereof. In other preferred embodiments, the isolated nucleic acid molecule encodes one of the amino acid sequences set forth as an even-numbered SEQ ID NO in the Sequence Listing (SEQ ID NO:2, SEQ ID NO:4). The preferred MP proteins of the present invention also preferably possess at least one of the MP activities described herein.

In another embodiment, the isolated nucleic acid molecule encodes a protein or portion thereof wherein the protein or portion thereof includes an amino acid sequence which is sufficiently homologous to an amino acid sequence of the invention (e.g., a sequence having an even-numbered SEQ ID NO: in the Sequence Listing), e.g., sufficiently homologous to an amino acid sequence of the invention such that the protein or portion thereof maintains an MP activity. Preferably, the protein or portion thereof encoded by the nucleic acid molecule maintains the ability to perform an enzymatic reaction in a amino acid, vitamin, cofactor, nutraceutical, nucleotide, nucleoside, or trehalose metabolic pathway. In one embodiment, the protein encoded by the nucleic acid molecule is at least about 63%, preferably at least about 71%, and more preferably at least about 75%, 80%, or 90% and most preferably at least about 95%, 96%, 97%, 98%, or 99% or more homologous to an amino acid sequence of the invention (e.g., an entire amino acid sequence selected from those having an even-numbered SEQ ID NO in the Sequence Listing). In another preferred embodiment, the protein is a full length C. glutamicum protein which is substantially homologous to an entire amino acid sequence of the invention (encoded by an open reading frame shown in the corresponding odd-numbered SEQ ID NOs in the Sequence Listing (SEQ ID NO:2, SEQ ID NO:4).

In another preferred embodiment, the isolated nucleic acid molecule is derived from C. glutamicum and encodes a protein (e.g., an MP fusion protein) which includes a biologically active domain which is at least about 50% or more homologous to one of the amino acid sequences of the invention (e.g., a sequence of one of the even-numbered SEQ ID NOs in the Sequence Listing) and is able to catalyze a reaction in a metabolic pathway for an amino acid, vitamin, cofactor, nutraceutical, nucleotide, nucleoside, or trehalose, or one or more of the activities set forth in Table 1, and which also includes heterologous nucleic acid sequences encoding a heterologous polypeptide or regulatory regions.

In another embodiment, the isolated nucleic acid molecule is at least 15 nucleotides in length and hybridizes under stringent conditions to a nucleic acid molecule comprising a nucleotide sequence of the invention (e.g., a sequence of an odd-numbered SEQ ID NO in the Sequence Listing). Preferably, the isolated nucleic acid molecule corresponds to a naturally-occurring nucleic acid molecule. More preferably, the isolated nucleic acid encodes a naturally-occurring C. glutamicum MP protein, or a biologically active portion thereof.

Another aspect of the invention pertains to vectors, e.g., recombinant expression vectors, containing the nucleic acid molecules of the invention, and host cells into which such vectors have been introduced. In one embodiment, such a host cell is used to produce an MP protein by culturing the host cell in a suitable medium. The MP protein can be then isolated from the medium or the host cell.

Yet another aspect of the invention pertains to a genetically altered microorganism in which an MP gene has been introduced or altered. In one embodiment, the genome of the microorganism has been altered by introduction of a nucleic acid molecule of the invention encoding wild-type or mutated MP sequence as a transgene. In another embodiment, an endogenous MP gene within the genome of the microorganism has been altered, e.g., functionally disrupted, by homologous recombination with an altered MP gene. In another embodiment, an endogenous or introduced MP gene in a microorganism has been altered by one or more point mutations, deletions, or inversions, but still encodes a functional MP protein. In still another embodiment, one or more of the regulatory regions (e.g., a promoter, repressor, or inducer) of an MP gene in a microorganism has been altered (e.g., by deletion, truncation, inversion, or point mutation) such that the expression of the MP gene is modulated. In a preferred embodiment, the microorganism belongs to the genus Corynebacterium or Brevibacterium, with Corynebacterium glutamicum being particularly preferred. In a preferred embodiment, the microorganism is also utilized for the production of a desired compound, such as trehalose or an amino acid, with lysine and methionine being particularly preferred.

In another aspect, the invention provides a method of identifying the presence or activity of Cornyebacterium diphtheriae in a subject. This method includes detection of one or more of the nucleic acid or amino acid sequences of the invention (e.g., the sequences set forth in the Sequence Listing as SEQ ID NOs 1 through 4) in a subject, thereby detecting the presence or activity of Corynebacterium diphtheriae in the subject.

Still another aspect of the invention pertains to an isolated MP protein or a portion, e.g., a biologically active portion, thereof. In a preferred embodiment, the isolated MP protein or portion thereof can catalyze an enzymatic reaction involved in one or more pathways for the metabolism of an amino acid, a vitamin, a cofactor, a nutraceutical, a nucleotide, a nucleoside, or trehalose. In another preferred embodiment, the isolated MP protein or portion thereof is sufficiently homologous to an amino acid sequence of the invention (e.g., a sequence of an even-numbered SEQ ID NO: in the Sequence Listing) such that the protein or portion thereof maintains the ability to catalyze an enzymatic reaction involved in one or more pathways for the metabolism of an amino acid, a vitamin, a cofactor, a nutraceutical, a nucleotide, a nucleoside, or trehalose.

The invention also provides an isolated preparation of an MP protein. In preferred embodiments, the MP protein comprises an amino acid sequence of the invention (e.g., a sequence of an even-numbered SEQ ID NO: of the Sequence Listing). In another preferred embodiment, the invention pertains to an isolated full length protein which is substantially homologous to an entire amino acid sequence of the invention (e.g., a sequence of an even-numbered SEQ ID NO: of the Sequence Listing) (encoded by an open reading frame set forth in a corresponding odd-numbered SEQ ID NO: of the Sequence Listing). In yet another embodiment, the protein is at least about 63%, preferably at least about 71%, and more preferably at least about 75%, 80%, or 90%, and most preferably at least about 95%, 96%, 97%, 98%, or 99% or more homologous to an entire amino acid sequence of the invention (e.g., a sequence of an even-numbered SEQ ID NO: of the Sequence Listing). In other embodiments, the isolated MP protein comprises an amino acid sequence which is at least about 63% or more homologous to one of the amino acid sequences of the invention (e.g., a sequence of an even-numbered SEQ ID NO: of the Sequence Listing) and is able to catalyze an enzymatic reaction in an amino acid, vitamin, cofactor, nutraceutical, nucleotide, nucleoside, or trehalose metabolic pathway, or has one or more of the activities set forth in Table 1.

Alternatively, the isolated MP protein can comprise an amino acid sequence which is encoded by a nucleotide sequence which hybridizes, e.g., hybridizes under stringent conditions, or is at least about 63%, preferably at least about 71%, more preferably at least about 70%, 80%, or 90%, and even more preferably at least about 95%, 96%, 97%, 98,%, or 99% or more homologous to a nucleotide sequence encoding a proteine of one of the even-numbered SEQ ID NOs set forth in the Sequence Listing. It is also preferred that the preferred forms of MP proteins also have one or more of the MP bioactivities described herein.

The MP polypeptide, or a biologically active portion thereof, can be operatively linked to a non-MP polypeptide to form a fusion protein. In preferred embodiments, this fusion protein has an activity which differs from that of the MP protein alone. In other preferred embodiments, this fusion protein, when introduced into a C. glutamicum pathway for the metabolism of an amino acid, vitamin, cofactor, nutraceutical, results in increased yields and/or efficiency of production of a desired fine chemical from C. glutamicum. In particularly preferred embodiments, integration of this fusion protein into an amino acid, vitamin, cofactor, nutraceutical, nucleotide, nucleoside, or trehalose metabolic pathway of a host cell modulates production of a desired compound from the cell.

In another aspect, the invention provides methods for screening molecules which modulate the activity of an MP protein, either by interacting with the protein itself or a substrate or binding partner of the MP protein, or by modulating the transcription or translation of an MP nucleic acid molecule of the invention.

Another aspect of the invention pertains to a method for producing a fine chemical. This method involves the culturing of a cell containing a vector directing the expression of an MP nucleic acid molecule of the invention, such that a fine chemical is produced. In a preferred embodiment, this method further includes the step of obtaining a cell containing such a vector, in which a cell is transfected with a vector directing the expression of an MP nucleic acid. In another preferred embodiment, this method further includes the step of recovering the fine chemical from the culture. In a particularly preferred embodiment, the cell is from the genus Corynebacterium or Brevibacterium, or is selected from those strains set forth in Table 2.

TABLE 2
Corynebacterium and Brevibacterium strains which may
be used in the practice of the invention

| Genus | Species | ATCC | FERM | NRRL | CECT | NCIMB | CBS | NCTC | DSMZ |
|---|---|---|---|---|---|---|---|---|---|
| Brevibacterium | Ammoniagenes | 21054 | | | | | | | |
| Brevibacterium | Ammoniagenes | 19350 | | | | | | | |
| Brevibacterium | Ammoniagenes | 19351 | | | | | | | |
| Brevibacterium | Ammoniagenes | 19352 | | | | | | | |
| Brevibacterium | Ammoniagenes | 19353 | | | | | | | |
| Brevibacterium | Ammoniagenes | 19354 | | | | | | | |
| Brevibacterium | Ammoniagenes | 19355 | | | | | | | |
| Brevibacterium | Ammoniagenes | 19356 | | | | | | | |
| Brevibacterium | Ammoniagenes | 21055 | | | | | | | |
| Brevibacterium | Ammoniagenes | 21077 | | | | | | | |
| Brevibacterium | Ammoniagenes | 21553 | | | | | | | |
| Brevibacterium | Ammoniagenes | 21580 | | | | | | | |
| Brevibacterium | Ammoniagenes | 39101 | | | | | | | |
| Brevibacterium | Butanicum | 21196 | | | | | | | |
| Brevibacterium | Divaricatum | 21792 | P928 | | | | | | |
| Brevibacterium | Flavum | 21474 | | | | | | | |
| Brevibacterium | Flavum | 21129 | | | | | | | |
| Brevibacterium | Flavum | 21518 | | | | | | | |
| Brevibacterium | Flavum | | | B11474 | | | | | |
| Brevibacterium | Flavum | | | B11472 | | | | | |
| Brevibacterium | Flavum | 21127 | | | | | | | |
| Brevibacterium | Flavum | 21128 | | | | | | | |
| Brevibacterium | Flavum | 21427 | | | | | | | |
| Brevibacterium | Flavum | 21475 | | | | | | | |
| Brevibacterium | Flavum | 21517 | | | | | | | |
| Brevibacterium | Flavum | 21528 | | | | | | | |
| Brevibacterium | Flavum | 21529 | | | | | | | |
| Brevibacterium | Flavum | | | B11477 | | | | | |
| Brevibacterium | Flavum | | | B11478 | | | | | |
| Brevibacterium | Flavum | 21127 | | | | | | | |
| Brevibacterium | Flavum | | | B11474 | | | | | |
| Brevibacterium | Healii | 15527 | | | | | | | |
| Brevibacterium | Ketoglutamicum | | 21004 | | | | | | |
| Brevibacterium | Ketoglutamicum | 21089 | | | | | | | |
| Brevibacterium | Ketosoreductum | 21914 | | | | | | | |
| Brevibacterium | Lactofermentum | | | | 70 | | | | |
| Brevibacterium | Lactofermentum | | | | 74 | | | | |
| Brevibacterium | Lactofermentum | | | | 77 | | | | |
| Brevibacterium | Lactofermentum | 21798 | | | | | | | |
| Brevibacterium | Lactofermentum | 21799 | | | | | | | |
| Brevibacterium | Lactofermentum | 21800 | | | | | | | |
| Brevibacterium | Lactofermentum | 21801 | | | | | | | |
| Brevibacterium | Lactofermentum | | | B11470 | | | | | |
| Brevibacterium | Lactofermentum | | | B11471 | | | | | |
| Brevibacterium | Lactofermentum | 21086 | | | | | | | |
| Brevibacterium | Lactofermentum | 21420 | | | | | | | |
| Brevibacterium | Lactofermentum | 21086 | | | | | | | |
| Brevibacterium | Lactofermentum | 31269 | | | | | | | |
| Brevibacterium | Linens | 9174 | | | | | | | |
| Brevibacterium | Linens | 19391 | | | | | | | |
| Brevibacterium | Linens | 8377 | | | | | | | |
| Brevibacterium | Paraffinolyticum | | | | | 11160 | | | |
| Brevibacterium | spec. | | | | | | 717.73 | | |
| Brevibacterium | spec. | | | | | | 717.73 | | |
| Brevibacterium | spec. | 14604 | | | | | | | |
| Brevibacterium | spec. | 21860 | | | | | | | |
| Brevibacterium | spec. | 21864 | | | | | | | |
| Brevibacterium | spec. | 21865 | | | | | | | |
| Brevibacterium | spec. | 21866 | | | | | | | |
| Brevibacterium | spec. | 19240 | | | | | | | |
| Coryne- | Acetoacido- | 21476 | | | | | | | |
| bacterium | philum | | | | | | | | |
| Coryne- | Acetoacido- | 13870 | | | | | | | |
| bacterium | philum | | | | | | | | |
| Coryne- | Aceto- | | B11473 | | | | | | |
| bacterium | glutamicum | | | | | | | | |
| Coryne- | Aceto- | | B11475 | | | | | | |
| bacterium | glutamicum | | | | | | | | |
| Coryne- | Aceto- | | | | | | | | |
| bacterium | glutamicum | 15806 | | | | | | | |
| Coryne- | Aceto- | | | | | | | | |
| bacterium | glutamicum | 21491 | | | | | | | |
| Coryne- | Aceto- | | | | | | | | |
| bacterium | glutamicum | 31270 | | | | | | | |
| Coryne- | Acetophilum | | | B3671 | | | | | |
| bacterium | | | | | | | | | |
| Coryne- | Ammoniagenes | 6872 | | | | | | 2399 | |
| bacterium | | | | | | | | | |
| Coryne- | Ammoniagenes | 15511 | | | | | | | |
| bacterium | | | | | | | | | |
| Coryne- | Fujiokense | 21496 | | | | | | | |
| bacterium | | | | | | | | | |
| Coryne- | Glutamicum | 14067 | | | | | | | |
| bacterium | | | | | | | | | |
| Coryne- | Glutamicum | 39137 | | | | | | | |
| bacterium | | | | | | | | | |
| Coryne- | Glutamicum | 21254 | | | | | | | |
| bacterium | | | | | | | | | |
| Coryne- | Glutamicum | 21255 | | | | | | | |
| bacterium | | | | | | | | | |
| Coryne- | Glutamicum | 31830 | | | | | | | |
| bacterium | | | | | | | | | |
| Coryne- | Glutamicum | 13032 | | | | | | | |
| bacterium | | | | | | | | | |
| Coryne- | Glutamicum | 14305 | | | | | | | |
| bacterium | | | | | | | | | |
| Coryne- | Glutamicum | 15455 | | | | | | | |
| bacterium | | | | | | | | | |
| Coryne- | Glutamicum | 13058 | | | | | | | |
| bacterium | | | | | | | | | |
| Coryne | Glutamicum | 13059 | | | | | | | |
| bacterium | | | | | | | | | |
| Coryne- | Glutamicum | 13060 | | | | | | | |
| bacterium | | | | | | | | | |
| Coryne- | Glutamicum | 21492 | | | | | | | |
| bacterium | | | | | | | | | |
| Coryne- | Glutamicum | 21513 | | | | | | | |
| bacterium | | | | | | | | | |
| Coryne- | Glutamicum | 21526 | | | | | | | |
| bacterium | | | | | | | | | |
| Coryne- | Glutamicum | 21543 | | | | | | | |
| bacterium | | | | | | | | | |
| Coryne- | Glutamicum | 13287 | | | | | | | |
| bacterium | | | | | | | | | |
| Coryne- | Glutamicum | 21851 | | | | | | | |
| bacterium | | | | | | | | | |
| Coryne- | Glutamicum | 21253 | | | | | | | |
| bacterium | | | | | | | | | |
| Coryne- | glutamicum | 21514 | | | | | | | |
| bacterium | | | | | | | | | |
| Coryne- | glutamicum | 21516 | | | | | | | |
| bacterium | | | | | | | | | |
| Coryne- | glutamicum | 21299 | | | | | | | |
| bacterium | | | | | | | | | |
| Coryne- | glutamicum | 21300 | | | | | | | |
| bacterium | | | | | | | | | |
| Coryne- | glutamicum | 39684 | | | | | | | |
| bacterium | | | | | | | | | |
| Coryne- | glutamicum | 21488 | | | | | | | |
| bacterium | | | | | | | | | |
| Coryne- | glutamicum | 21649 | | | | | | | |
| bacterium | | | | | | | | | |
| Coryne- | glutamicum | 21650 | | | | | | | |
| bacterium | | | | | | | | | |
| Coryne- | glutamicum | 19223 | | | | | | | |
| bacterium | | | | | | | | | |
| Coryne- | glutamicum | 13869 | | | | | | | |
| bacterium | | | | | | | | | |
| Coryne- | glutamicum | 21157 | | | | | | | |
| bacterium | | | | | | | | | |
| Coryne- | glutamicum | 21158 | | | | | | | |
| bacterium | | | | | | | | | |
| Coryne- | glutamicum | 21159 | | | | | | | |
| bacterium | | | | | | | | | |
| Coryne- | glutamicum | 21355 | | | | | | | |
| bacterium | | | | | | | | | |
| Coryne- | glutamicum | 31808 | | | | | | | |
| bacterium | | | | | | | | | |
| Coryne- | glutamicum | 21674 | | | | | | | |
| bacterium | | | | | | | | | |
| Coryne- | glutamicum | 21562 | | | | | | | |
| bacterium | | | | | | | | | |
| Coryne- | glutamicum | 21563 | | | | | | | |
| bacterium | | | | | | | | | |
| Coryne- | glutamicum | 21564 | | | | | | | |
| bacterium | | | | | | | | | |
| Coryne- | glutamicum | 21565 | | | | | | | |
| bacterium | | | | | | | | | |
| Coryne- | glutamicum | 21566 | | | | | | | |
| bacterium | | | | | | | | | |
| Coryne- | glutamicum | 21567 | | | | | | | |
| bacterium | | | | | | | | | |
| Coryne- | glutamicum | 21568 | | | | | | | |
| bacterium | | | | | | | | | |
| Coryne- | glutamicum | 21569 | | | | | | | |
| bacterium | | | | | | | | | |
| Coryne- | glutamicum | 21570 | | | | | | | |
| bacterium | | | | | | | | | |
| Coryne- | glutamicum | 21571 | | | | | | | |
| bacterium | | | | | | | | | |
| Coryne- | glutamicum | 21572 | | | | | | | |
| bacterium | | | | | | | | | |
| Coryne- | glutamicum | 21573 | | | | | | | |
| bacterium | | | | | | | | | |
| Coryne- | glutamicum | 21579 | | | | | | | |
| bacterium | | | | | | | | | |
| Coryne- | glutamicum | 19049 | | | | | | | |
| bacterium | | | | | | | | | |
| Coryne- | glutamicum | 19050 | | | | | | | |
| bacterium | | | | | | | | | |
| Coryne- | glutamicum | 19051 | | | | | | | |
| bacterium | | | | | | | | | |
| Coryne- | glutamicum | 19052 | | | | | | | |
| bacterium | | | | | | | | | |
| Coryne- | glutamicum | 19053 | | | | | | | |
| bacterium | | | | | | | | | |
| Coryne- | glutamicum | 19054 | | | | | | | |
| bacterium | | | | | | | | | |
| Coryne- | glutamicum | 19055 | | | | | | | |
| bacterium | | | | | | | | | |
| Coryne- | glutamicum | 19056 | | | | | | | |
| bacterium | | | | | | | | | |
| Coryne- | glutamicum | 19057 | | | | | | | |
| bacterium | | | | | | | | | |
| Coryne- | glutamicum | 19058 | | | | | | | |
| bacterium | | | | | | | | | |
| Coryne- | glutamicum | 19059 | | | | | | | |
| bacterium | | | | | | | | | |
| Coryne- | glutamicum | 19060 | | | | | | | |
| bacterium | | | | | | | | | |
| Coryne- | glutamicum | 19185 | | | | | | | |
| bacterium | | | | | | | | | |
| Coryne- | glutamicum | 13286 | | | | | | | |
| bacterium | | | | | | | | | |
| Coryne- | glutamicum | 21515 | | | | | | | |
| bacterium | | | | | | | | | |
| Coryne- | glutamicum | 21527 | | | | | | | |
| bacterium | | | | | | | | | |
| Coryne- | glutamicum | 21544 | | | | | | | |
| bacterium | | | | | | | | | |
| Coryne- | glutamicum | 21492 | | | | | | | |
| bacterium | | | | | | | | | |
| Coryne | glutamicum | | | B8183 | | | | | |
| bacterium | | | | | | | | | |
| Coryne- | glutamicum | | | B8182 | | | | | |
| bacterium | | | | | | | | | |
| Coryne- | glutamicum | | | B12416 | | | | | |
| bacterium | | | | | | | | | |
| Coryne- | glutamicum | | | B12417 | | | | | |
| bacterium | | | | | | | | | |
| Coryne- | glutamicum | | | B12418 | | | | | |
| bacterium | | | | | | | | | |
| Coryne- | glutamicum | | | B11476 | | | | | |
| bacterium | | | | | | | | | |
| Coryne- | glutamicum | 21608 | | | | | | | |
| bacterium | | | | | | | | | |
| Coryne- | lilium | | P973 | | | | | | |
| bacterium | | | | | | | | | |
| Coryne- | nitrilophilus | 21419 | | | | 11594 | | | |
| bacterium | | | | | | | | | |
| Coryne- | spec. | | P4445 | | | | | | |
| bacterium | | | | | | | | | |
| Coryne- | spec. | | P4446 | | | | | | |
| bacterium | | | | | | | | | |
| Coryne- | spec. | 31088 | | | | | | | |
| bacterium | | | | | | | | | |
| Coryne- | spec. | 31089 | | | | | | | |
| bacterium | | | | | | | | | |
| Coryne- | spec. | 31090 | | | | | | | |
| bacterium | | | | | | | | | |
| Coryne- | spec. | 31090 | | | | | | | |
| bacterium | | | | | | | | | |
| Coryne- | spec. | 31090 | | | | | | | |
| bacterium | | | | | | | | | |
| Coryne- | spec. | 15954 | | | | | | | 20145 |
| bacterium | | | | | | | | | |
| Coryne- | spec. | 21857 | | | | | | | |
| bacterium | | | | | | | | | |
| Coryne- | spec. | 21862 | | | | | | | |
| bacterium | | | | | | | | | |
| Coryne- | spec. | 21863 | | | | | | | |
| bacterium | | | | | | | | | |

ATCC: American Type Culture Collection, Rockville, ND, U.S.A.
FERM: Fermentation Research Institute, Chiba, Japan
NRRL: ARS Culture Collection, Northern Regional Research Laboratory, Peoria, IL, U.S.A.
CECT: Coleccion Espanola de Cultivos Tipo, Valencia, Spain
NCIMB: National Collection of Industrial and Marine Bacteria Ltd., Aberdeen, U.K.
CBS: Centraalbureau voor Schimmelcultures, Baarn, NL
NCTC: National Collection of Type Cultures, London, U.K.
DSMZ: Deutsche Saromlung von Mikroorganismen und Zellkulturen, Braunschweig, Germany

Another aspect of the invention pertains to methods for modulating production of a molecule from a microorganism. Such methods include contacting the cell with an agent which modulates MP protein activity or MP nucleic acid expression such that a cell associated activity is altered relative to this same activity in the absence of the agent. In a preferred embodiment, the cell is modulated for one or more C. glutamicum amino acid, vitamin, cofactor, nutraceutical, nucleotide, nucleoside, or trehalose metabolic pathways, such that the yields or rate of production of a desired fine chemical by this microorganism is improved. The agent which modulates MP protein activity can be an agent which stimulates MP protein activity or MP nucleic acid expression. Examples of agents which stimulate MP protein activity or MP nucleic acid expression include small molecules, active MP proteins, and nucleic acids encoding MP proteins that have been introduced into the cell. Examples of agents which inhibit MP activity or expression include small molecules, and antisense MP nucleic acid molecules.

Another aspect of the invention pertains to methods for modulating yields of a desired compound from a cell, involving the introduction of a wild-type or mutant MP gene into a cell, either maintained on a separate plasmid or integrated into the genome of the host cell. If integrated into the genome, such integration can be random, or it can take place by homologous recombination such that the native gene is replaced by the introduced copy, causing the production of the desired compound from the cell to be modulated. In a preferred embodiment, said yields are increased. In another preferred embodiment, said chemical is a fine chemical. In a particularly preferred embodiment, said fine chemical is trehalose or an amino acid. In especially preferred embodiments, said amino acid are L-lysine and L-methionine.

### DETAILED DESCRIPTION OF THE INVENTION

The present invention provides MP nucleic acid and protein molecules which are involved in the metabolism of certain fine chemicals in Corynebacterium glutamicum, including amino acids, vitamins, cofactors, nutraceuticals, nucleotides, nucleosides, and trehalose. The molecules of the invention may be utilized in the modulation of production of fine chemicals from microorganisms, such as C. glutamicum, either directly (e.g., where modulation of the activity of a trehalose or a lysine or methionine biosynthesis protein has a direct impact on the production or efficiency of production of trehalose or lysine or methionine from that organism), or may have an indirect impact which nonetheless results in an increase of yield or efficiency of production of the desired compound (e.g., where modulation of the activity of a nucleotide biosynthesis protein has an impact on the production of an organic acid or a fatty acid from the bacterium, perhaps due to improved growth or an increased supply of necessary co-factors, energy compounds, or precursor molecules). Aspects of the invention are further explicated below.

1. Fine Chemicals

The term ‘fine chemical’ is art-recognized and includes molecules produced by an organism which have applications in various industries, such as, but not limited to, the pharmaceutical, agriculture, and cosmetics industries. Such compounds include organic acids, such as tartaric acid, itaconic acid, and diaminopimelic acid, both proteinogenic and non-proteinogenic amino acids, purine and pyrimidine bases, nucleosides, and nucleotides (as described e.g. in Kuninaka, A. (1996) Nucleotides and related compounds, p. 561-612, in Biotechnology vol. 6, Rehm et al., eds. VCH: Weinheim, and references contained therein), lipids, both saturated and unsaturated fatty acids (e.g., arachidonic acid), diols (e.g., propane diol, and butane diol), carbohydrates (e.g., hyaluronic acid and trehalose), aromatic compounds (e.g., aromatic amines, vanillin, and indigo), vitamins and cofactors (as described in Ullmann's Encyclopedia of Industrial Chemistry, vol. A27, “Vitamins”, p. 443-613 (1996) VCH: Weinheim and references therein; and Ong, A. S., Niki, E. & Packer, L. (1995) “Nutrition, Lipids, Health, and Disease” Proceedings of the UNESCO/Confederation of Scientific and Technological Associations in Malaysia, and the Society for Free Radical Research—Asia, held Sep. 1-3, 1994 at Penang, Malaysia, AOCS Press, (1995)), enzymes, polyketides (Cane et al. (1998) Science 282: 63-68), and all other chemicals described in Gutcho (1983) Chemicals by Fermentation, Noyes Data Corporation, ISBN: 0818805086 and references therein. The metabolism and uses of certain of these fine chemicals are further explicated below.

A. Amino Acid Metabolism and Uses

Amino acids comprise the basic structural units of all proteins, and as such are essential for normal cellular functioning in all organisms. The term “amino acid” is art-recognized. The proteinogenic amino acids, of which there are 20 species, serve as structural units for proteins, in which they are linked by peptide bonds, while the nonproteinogenic amino acids (hundreds of which are known) are not normally found in proteins (see Ulmann's Encyclopedia of Industrial Chemistry, vol. A2, p. 57-97 VCH: Weinheim (1985)). Amino acids may be in the D- or L-optical configuration, though L-amino acids are generally the only type found in naturally-occurring proteins. Biosynthetic and degradative pathways of each of the 20 proteinogenic amino acids have been well characterized in both prokaryotic and eukaryotic cells (see, for example, Stryer, L. Biochemistry, 3^{rd} edition, pages 578-590 (1988)). The ‘essential’ amino acids (histidine, isoleucine, leucine, lysine, methionine, phenylalanine, threonine, tryptophan, and valine), so named because they are generally a nutritional requirement due to the complexity of their biosyntheses, are readily converted by simple biosynthetic pathways to the remaining 11 ‘nonessential’ amino acids (alanine, arginine, asparagine, aspartate, cysteine, glutamate, glutamine, glycine, proline, serine, and tyrosine). Higher animals do retain the ability to synthesize some of these amino acids, but the essential amino acids must be supplied from the diet in order for normal protein synthesis to occur.

Aside from their function in protein biosynthesis, these amino acids are interesting chemicals in their own right, and many have been found to have various applications in the food, feed, chemical, cosmetics, agriculture, and pharmaceutical industries. Lysine is an important amino acid in the nutrition not only of humans, but also of monogastric animals such as poultry and swine. Glutamate is most commonly used as a flavor additive (mono-sodium glutamate, MSG) and is widely used throughout the food industry, as are aspartate, phenylalanine, glycine, and cysteine. Glycine, L-methionine and tryptophan are all utilized in the pharmaceutical industry. Glutamine, valine, leucine, isoleucine, histidine, arginine, proline, serine and alanine are of use in both the pharmaceutical and cosmetics industries. Threonine, tryptophan, and D/L-methionine are common feed additives. (Leuchtenberger, W. (1996) Amino aids—technical production and use, p. 466-502 in Rehm et al. (eds.) Biotechnology vol. 6, chapter 14a, VCH: Weinheim). Additionally, these amino acids have been found to be useful as precursors for the synthesis of synthetic amino acids and proteins, such as N-acetylcysteine, S-carboxymethyl-L-cysteine, (S)-5-hydroxytryptophan, and others described in Ulmann's Encyclopedia of Industrial Chemistry, vol. A2, p. 57-97, VCH: Weinheim, 1985.

The biosynthesis of these natural amino acids in organisms capable of producing them, such as bacteria, has been well characterized (for review of bacterial amino acid biosynthesis and regulation thereof, see Umbarger, H. E. (1978) Ann. Rev. Biochem. 47: 533-606). Glutamate is synthesized by the reductive amination of á-ketoglutarate, an intermediate in the citric acid cycle. Glutamine, proline, and arginine are each subsequently produced from glutamate. The biosynthesis of serine is a three-step process beginning with 3-phosphoglycerate (an intermediate in glycolysis), and resulting in this amino acid after oxidation, transamination, and hydrolysis steps. Both cysteine and glycine are produced from serine; the former by the condensation of homocysteine with serine, and the latter by the transferal of the side-chain â-carbon atom to tetrahydrofolate, in a reaction catalyzed by serine transhydroxymethylase. Phenylalanine, and tyrosine are synthesized from the glycolytic and pentose phosphate pathway precursors erythrose 4-phosphate and phosphoenolpyruvate in a 9-step biosynthetic pathway that differ only at the final two steps after synthesis of prephenate. Tryptophan is also produced from these two initial molecules, but its synthesis is an 11-step pathway. Tyrosine may also be synthesized from phenylalanine, in a reaction catalyzed by phenylalanine hydroxylase. Alanine, valine, and leucine are all biosynthetic products of pyruvate, the final product of glycolysis. Aspartate is formed from oxaloacetate, an intermediate of the citric acid cycle. Asparagine, methionine, threonine, and lysine are each produced by the conversion of aspartate. Isoleucine is formed from threonine. The biosynthetic pathways leading to methionine have been studied in diverse organisms and show similarity as well as differences. The first step, acylation of homoserine, is common to all the organisms, even though the source of the transferred acyl groups is different. Escherichia coli and the related species use succinyl-CoA (Michaeli, S. and Ron, E. Z. (1981) Construction and physical mapping of plasmids containing the metA gene of Escherichia coli K12, Mol. Gen. Genet. 182, 349-354). Construction and physical mapping of plasmids containing the metA gene of Escherichia coli K12, Mol. Gen. Genet. 182, 349-354), while Saccharomyces cerevisiae (Langin, T., Faugeron, G., Goyon, C., Nicolas, A., and Rossignol, J. (1986) The MET2 gene of Saccharomyces cerevisiae: molecular cloning and nucleotide sequence. Gene 49, 283-293), Brevibacterium flavum (Miyajima, R. and Shiio, I. (1973) Regulation of aspartate family of amino acid biosynthesis in Brevibacterium flavum: properties of homoserine O-transacetylase. J. Biochem. 73, 1061-1068; Ozaki, H. and Shiio, I. (1982) Methionine biosynthesis in Brevibacterium flavum: properties and essential role of O-acetylhomoserine sulfhydrylase. J. Biochem. 91, 1163-1171), C. glutamicum (Park, S.-D., Lee, J.-Y., Kim, Y., Kim, J.-H., and Lee, H.-S. (1998) Isolation and analysis of metA, a methionine biosynthetic gene encoding homoserine acetyltransferase in Corynebacterium glutamicum. Mol. Cells 8, 286-294), and Leptospira meyeri (Belfaiza, J., Martel, A., Maegarita. D., and Saint Girons, I. (1998) Direct sulfhydrylation for methionine biosynthesis in Leptospira meyeri. J. Bacteriol. 180, 250-255; Bourhy, P., Martel, A., Margarita, D., Saint Girons, I., and Belfaiza, J. (1997) Homoserine O-acetyltransferase, involved in the Leptospira meyeri methionine biosynthetic pathway, is not feedback inhibited. J. Bacteriol. 179, 4396-4398) use acetyl-CoA as the acyl donor. Formation of homocysteine from acylhomoserine can occur in two different ways. E. coli uses the transsulfuration pathway which is catalyzed by cystathionine γ-synthase (the product of metB) and cystathionine β-lyase (the product of metC). S. cerevisiae (Cherest, H. and Surdin-Kerjan, Y. (1992) Genetic analysis of a new mutation conferring cysteine auxotrophy in Saccharomyces cerevisiae: updating of the sulfur metabolism pathway. Genetics 130, 51-58), B. flavum (Ozaki, H. and Shiio, I. (1982) Methionine biosynthesis in Brevibacterium flavum: properties and essential role of O-acetylhomoserine sulfhydrylase. J. Biochem. 91, 1163-1171), Pseudomonas aeruginosa (Foglino, M., Borne, F., Bally, M., Ball, G., and Patte, J. C. (1995) A direct sulfhydrylation pathway is used for methionine biosynthesis in Pseudomonas aeruginosa. Microbiology 141, 431-439), and L. meyeri (Belfaiza, J., Martel, A., Maegarita. D., and Saint Girons, I. (1998) Direct sulfhydrylation for methionine biosynthesis in Leptospira meyeri. J. Bacteriol. 180, 250-255) utilize the direct sulfhydrylation pathway which is catalyzed by acylhomoserine sulfhydrylase. Unlike closely related B. flavum which uses only the direct sulfhydrylation pathway, enzyme activities of the transsulfuration pathway have been detected in the extracts of the C. glutamicum cells and the pathway has been proposed to be the route for methionine biosynthesis in the organism (Hwang, B-J., Kim, Y., Kim, H.-B., Kim, J., Hwang, H.-J., and Lee, H.-S. (1999) Analysis of Corynebacterium glutamicum methionine biosynthetic pathway: Isolation and analysis of metB encoding cystathionine ã-synthase. Mol. Cells 9, 300-308; Kase, H. and Nakayama, K. (1974) Production of O-acetyl-L-homoserine by methionine analog resistant mutants and regulation of homoserine-O-transacetylase in Corynebacterium glutamicum. Agr. Biol. Chem. 38, 2021-2030; Park, S.-D., Lee, J.-Y., Kim, Y., Kim, J.-H., and Lee, H.-S. (1998) Isolation and analysis of metA, a methionine biosynthetic gene encoding homoserine acetyltransferase in Corynebacterium glutamicum. Mol. Cells 8, 286-294).

Even though some genes involved in methionine biosynthesis in C. glutamicum were isolated in recent years, the information on the biosynthesis of methionine in C. glutamicum is still limited. The metA and metB genes have been isolated from the organism and also the metC and the metZ gene are known (table 4), but the final step of the biosynthesis remained unclear. In this invention, the biosynthetic pathway leading to methionine in C. glutamicum is deciphered in total and the biosynthetic gene responsible for the last step of the biosynthesis is defined with the metH gene encoding the enzyme methionine synthase.

A complex 9-step pathway results in the production of histidine from 5-phosphoribosyl-1-pyrophosphate, an activated sugar.

Amino acids in excess of the protein synthesis needs of the cell cannot be stored, and are instead degraded to provide intermediates for the major metabolic pathways of the cell (for review see Stryer, L. Biochemistry 3^{rd} ed. Ch. 21 “Amino Acid Degradation and the Urea Cycle” p. 495-516 (1988)). Although the cell is able to convert unwanted amino acids into useful metabolic intermediates, amino acid production is costly in terms of energy, precursor molecules, and the enzymes necessary to synthesize them. Thus it is not surprising that amino acid biosynthesis is regulated by feedback inhibition, in which the presence of a particular amino acid serves to slow or entirely stop its own production (for overview of feedback mechanisms in amino acid biosynthetic pathways, see Stryer, L. Biochemistry, 3^{rd} ed. Ch. 24: “Biosynthesis of Amino Acids and Heme” p. 575-600 (1988)). Thus, the output of any particular amino acid is limited by the amount of that amino acid present in the cell.

2.1 Vitamin, Cofactor, and Nutraceutical Metabolism and Uses

Vitamins, cofactors, and nutraceuticals comprise another group of molecules which the higher animals have lost the ability to synthesize and so must ingest, although they are readily synthesized by other organisms, such as bacteria. These molecules are either bioactive substances themselves, or are precursors of biologically active substances which may serve as electron carriers or intermediates in a variety of metabolic pathways. Aside from their nutritive value, these compounds also have significant industrial value as coloring agents, antioxidants, and catalysts or other processing aids. (For an overview of the structure, activity, and industrial applications of these compounds, see, for example, Ullman's Encyclopedia of Industrial Chemistry, “Vitamins” vol. A27, p. 443-613, VCH: Weinheim, 1996.) The term “vitamin” is art-recognized, and includes nutrients which are required by an organism for normal functioning, but which that organism cannot synthesize by itself. The group of vitamins may encompass cofactors and nutraceutical compounds. The language “cofactor” includes nonproteinaceous compounds required for a normal enzymatic activity to occur. Such compounds may be organic or inorganic; the cofactor molecules of the invention are preferably organic. The term “nutraceutical” includes dietary supplements having health benefits in plants and animals, particularly humans. Examples of such molecules are vitamins, antioxidants, and also certain lipids (e.g., polyunsaturated fatty acids).

The biosynthesis of these molecules in organisms capable of producing them, such as bacteria, has been largely characterized (Ullman's Encyclopedia of Industrial Chemistry, “Vitamins” vol. A27, p. 443-613, VCH: Weinheim, 1996; Michal, G. (1999) Biochemical Pathways: An Atlas of Biochemistry and Molecular Biology, John Wiley & Sons; Ong, A. S., Niki, E. & Packer, L. (1995) “Nutrition, Lipids, Health, and Disease” Proceedings of the UNESCO/Confederation of Scientific and Technological Associations in Malaysia, and the Society for Free Radical Research—Asia, held Sep. 1-3, 1994 at Penang, Malaysia, AOCS Press: Champaign, Ill. X, 374 S).

Thiamin (vitamin B₁) is produced by the chemical coupling of pyrimidine and thiazole moieties. Riboflavin (vitamin B₂) is synthesized from guanosine-5′-triphosphate (GTP) and ribose-5′-phosphate. Riboflavin, in turn, is utilized for the synthesis of flavin mononucleotide (FMN) and flavin adenine dinucleotide (FAD). The family of compounds collectively termed ‘vitamin B₆’ (e.g., pyridoxine, pyridoxamine, pyridoxa-5′-phosphate, and the commercially used pyridoxin hydrochloride) are all derivatives of the common structural unit, 5-hydroxy-6-methylpyridine. Pantothenate (pantothenic acid, (R)-(+)—N-(2,4-dihydroxy-3,3-dimethyl-1-oxobutyl)-â-alanine) can be produced either by chemical synthesis or by fermentation. The final steps in pantothenate biosynthesis consist of the ATP-driven condensation of â-alanine and pantoic acid. The enzymes responsible for the biosynthesis steps for the conversion to pantoic acid, to â-alanine and for the condensation to panthotenic acid are known. The metabolically active form of pantothenate is Coenzyme A, for which the biosynthesis proceeds in 5 enzymatic steps. Pantothenate, pyridoxal-5′-phosphate, cysteine and ATP are the precursors of Coenzyme A. These enzymes not only catalyze the formation of panthothante, but also the production of (R)-pantoic acid, (R)-pantolacton, (R)-panthenol (provitamin B₅), pantetheine (and its derivatives) and coenzyme A.

Biotin biosynthesis from the precursor molecule pimeloyl-CoA in microorganisms has been studied in detail and several of the genes involved have been identified. Many of the corresponding proteins have been found to also be involved in Fe-cluster synthesis and are members of the nifS class of proteins. Lipoic acid is derived from octanoic acid, and serves as a coenzyme in energy metabolism, where it becomes part of the pyruvate dehydrogenase complex and the á-ketoglutarate dehydrogenase complex. The folates are a group of substances which are all derivatives of folic acid, which is turn is derived from L-glutamic acid, p-amino-benzoic acid and 6-methylpterin. The biosynthesis of folic acid and its derivatives, starting from metabolism intermediates guanosine-5′-triphosphate (GTP), L-glutamic acid and p-amino-benzoic acid has been studied in detail in certain microorganisms.

Corrinoids (such as the cobalamines and particularly vitamin B₁₂) and porphyrines belong to a group of chemicals characterized by a tetrapyrole ring system. The biosynthesis of vitamin B₁₂ is sufficiently complex that it has not yet been completely characterized, but many of the enzymes and substrates involved are now known. Nicotinic acid (nicotinate), and nicotinamide are pyridine derivatives which are also termed ‘niacin’. Niacin is the precursor of the important coenzymes NAD (nicotinamide adenine dinucleotide) and NADP (nicotinamide adenine dinucleotide phosphate) and their reduced forms.

The large-scale production of these compounds has largely relied on cell-free chemical syntheses, though some of these chemicals have also been produced by large-scale culture of microorganisms, such as riboflavin, Vitamin B₆, pantothenate, and biotin. Only Vitamin B₁₂ is produced solely by fermentation, due to the complexity of its synthesis. In vitro methodologies require significant inputs of materials and time, often at great cost.

C. Purine, Pyrimidine, Nucleoside and Nucleotide Metabolism and Uses

Purine and pyrimidine metabolism genes and their corresponding proteins are important targets for the therapy of tumor diseases and viral infections. The language “purine” or “pyrimidine” includes the nitrogenous bases which are constituents of nucleic acids, co-enzymes, and nucleotides. The term “nucleotide” includes the basic structural units of nucleic acid molecules, which are comprised of a nitrogenous base, a pentose sugar (in the case of RNA, the sugar is ribose; in the case of DNA, the sugar is D-deoxyribose), and phosphoric acid. The language “nucleoside” includes molecules which serve as precursors to nucleotides, but which are lacking the phosphoric acid moiety that nucleotides possess. By inhibiting the biosynthesis of these molecules, or their mobilization to form nucleic acid molecules, it is possible to inhibit RNA and DNA synthesis; by inhibiting this activity in a fashion targeted to cancerous cells, the ability of tumor cells to divide and replicate may be inhibited. Additionally, there are nucleotides which do not form nucleic acid molecules, but rather serve as energy stores (i.e., AMP) or as coenzymes (i.e., FAD and NAD).

Several publications have described the use of these chemicals for these medical indications, by influencing purine and/or pyrimidine metabolism (e.g. Christopherson, R. I. and Lyons, S. D. (1990) “Potent inhibitors of de novo pyrimidine and purine biosynthesis as chemotherapeutic agents.” Med. Res. Reviews 10: 505-548). Studies of enzymes involved in purine and pyrimidine metabolism have been focused on the development of new drugs which can be used, for example, as immunosuppressants or anti-proliferants (Smith, J. L., (1995) “Enzymes in nucleotide synthesis.” Curr. Opin. Struct. Biol. 5: 752-757; (1995) Biochem Soc. Transact. 23: 877-902). However, purine and pyrimidine bases, nucleosides and nucleotides have other utilities: as intermediates in the biosynthesis of several fine chemicals (e.g., thiamine, S-adenosyl-methionine, folates, or riboflavin), as energy carriers for the cell (e.g., ATP or GTP), and for chemicals themselves, commonly used as flavor enhancers (e.g., IMP or GMP) or for several medicinal applications (see, for example, Kuninaka, A. (1996) Nucleotides and Related Compounds in Biotechnology vol. 6, Rehm et al., eds. VCH: Weinheim, p. 561-612). Also, enzymes involved in purine, pyrimidine, nucleoside, or nucleotide metabolism are increasingly serving as targets against which chemicals for crop protection, including fungicides, herbicides and insecticides, are developed.

The metabolism of these compounds in bacteria has been characterized (for reviews see, for example, Zalkin, H. and Dixon, J. E. (1992) “de novo purine nucleotide biosynthesis”, in: Progress in Nucleic Acid Research and Molecular Biology, vol. 42, Academic Press:, p. 259-287; and Michal, G. (1999) “Nucleotides and Nucleosides”, Chapter 8 in: Biochemical Pathways: An Atlas of Biochemistry and Molecular Biology, Wiley: New York). Purine metabolism has been the subject of intensive research, and is essential to the normal functioning of the cell. Impaired purine metabolism in higher animals can cause severe disease, such as gout. Purine nucleotides are synthesized from ribose-5-phosphate, in a series of steps through the intermediate compound inosine-5′-phosphate (IMP), resulting in the production of guanosine-5′-monophosphate (GMP) or adenosine-5′-monophosphate (AMP), from which the triphosphate forms utilized as nucleotides are readily formed. These compounds are also utilized as energy stores, so their degradation provides energy for many different biochemical processes in the cell. Pyrimidine biosynthesis proceeds by the formation of uridine-5′-monophosphate (UMP) from ribose-5-phosphate. UMP, in turn, is converted to cytidine-5′-triphosphate (CTP) The deoxy-forms of all of these nucleotides are produced in a one step reduction reaction from the diphosphate ribose form of the nucleotide to the diphosphate deoxyribose form of the nucleotide. Upon phosphorylation, these molecules are able to participate in DNA synthesis.

D. Trehalose Metabolism and Uses

Trehalose consists of two glucose molecules, bound in á, á-1,1 linkage. It is commonly used in the food industry as a sweetener, an additive for dried or frozen foods, and in beverages. However, it also has applications in the pharmaceutical, cosmetics and biotechnology industries (see, for example, Nishimoto et al., (1998) U.S. Pat. No. 5,759,610; Singer, M. A. and Lindquist, S. (1998) Trends Biotech. 16: 460-467; Paiva, C. L. A. and Panek, A. D. (1996) Biotech. Ann. Rev. 2: 293-314; and Shiosaka, M. (1997) J. Japan 172: 97-102). Trehalose is produced by enzymes from many microorganisms and is naturally released into the surrounding medium, from which it can be collected using methods known in the art.

II. Elements and Methods of the Invention

The present invention is based, at least in part, on the discovery of novel molecules, referred to herein as MP nucleic acid and protein molecules, which play a role in or function in one or more cellular metabolic pathways. In one embodiment, the MP molecules catalyze an enzymatic reaction involving one or more amino acid, vitamin, cofactor, nutraceutical, nucleotide, nucleoside, or trehalose metabolic pathways. In a preferred embodiment, the activity of the MP molecules of the present invention in one or more C. glutamicum metabolic pathways for amino acids, vitamins, cofactors, nutraceuticals, nucleotides, nucleosides or trehalose has an impact on the production of a desired fine chemical by this organism. In a particularly preferred embodiment, the MP molecules of the invention are modulated in activity, such that the C. glutamicum metabolic pathways in which the MP proteins of the invention are involved are modulated in efficiency or output, which either directly or indirectly modulates the production or efficiency of production of a desired fine chemical by C. glutamicum. The MP molecules may be combined with other MP molecules of the same or different metabolic pathway to increase the yield of a desired fine chemical, preferred trehalose or an amino acid, more preferred lysine or methionine. Alternatively or in addition a byproduct which is not desired may be reduced by combination of disruption of MP molecules or other metabolic molecules. The MP molecules combined with other MP molecules of the same or a different pathway may be altered in their nucleotide and in the corresponding amino acid sequence in such a way that their activity is altered under physiological conditions which leads to an increase in productivity and/or yield of a desired fine chemical. In a further embodiment the MP molecule in its original or in its above described altered form may be combined with other MP molecules of the same or a different pathway wich are altered in their nucleotide sequence in such a way that their activity is altered under physiological conditions which leads to an increase in productivity and/or yield of a desired fine chemical. Preferred combinations are such which combine one ore both MP molecules of table1 with one ore more single or multipe copies of MP proteins of tables 4 and 5 or the respective published MP molecules of the same metabolic pathway (Methionine biosyntesis or trehalose/phosphoenolpyruvat way).

The language, “MP protein” or “MP polypeptide” includes proteins which play a role in, e.g., catalyze an enzymatic reaction, in one or more amino acid, vitamin, cofactor, nutraceutical, nucleotide, nucleoside or trehalose metabolic pathways. Examples of MP proteins include those encoded by the MP genes set forth in Table 1 and by the odd-numbered SEQ ID NOs. The terms “MP gene” or “MP nucleic acid sequence” include nucleic acid sequences encoding an MP protein, which consist of a coding region and also corresponding untranslated 5′ and 3′ sequence regions. Examples of MP genes include those set forth in Table 1. The terms “production” or “productivity” are art-recognized and include the concentration of the fermentation product (for example, the desired fine chemical) formed within a given time and a given fermentation volume (e.g., kg product per hour per liter). The term “efficiency of production” includes the time required for a particular level of production to be achieved (for example, how long it takes for the cell to attain a particular rate of output of a fine chemical). The term “yield” or “product/carbon yield” is art-recognized and includes the efficiency of the conversion of the carbon source into the product (i.e., fine chemical). This is generally written as, for example, kg product per kg carbon source. By increasing the yield or production of the compound, the quantity of recovered molecules, or of useful recovered molecules of that compound in a given amount of culture over a given amount of time is increased. The terms “biosynthesis” or a “biosynthetic pathway” are art-recognized and include the synthesis of a compound, preferably an organic compound, by a cell from intermediate compounds in what may be a multistep and highly regulated process. The terms “degradation” or a “degradation pathway” are art-recognized and include the breakdown of a compound, preferably an organic compound, by a cell to degradation products (generally speaking, smaller or less complex molecules) in what may be a multistep and highly regulated process. The language “metabolism” is art-recognized and includes the totality of the biochemical reactions that take place in an organism. The metabolism of a particular compound, then, (e.g., the metabolism of an amino acid such as glycine) comprises the overall biosynthetic, modification, and degradation pathways in the cell related to this compound.

In another embodiment, the MP molecules of the invention are capable of modulating the production of a desired molecule, such as a fine chemical, in a microorganism such as C. glutamicum. Using recombinant genetic techniques, one or more of the biosynthetic or degradative enzymes of the invention for amino acids, vitamins, cofactors, nutraceuticals, nucleotides, nucleosides, or trehalose may be manipulated such that its function is modulated. For example, a biosynthetic enzyme may be improved in efficiency, or its allosteric control region destroyed such that feedback inhibition of production of the compound is prevented. Similarly, a degradative enzyme may be deleted or modified by substitution, deletion, or addition such that its degradative activity is lessened for the desired compound without impairing the viability of the cell. In each case, the overall yield or rate of production of one of these desired fine chemicals may be increased.

It is also possible that such alterations in the protein and nucleotide molecules of the invention may improve the production of other fine chemicals besides the amino acids, vitamins, cofactors, nutraceuticals, nucleotides, nucleosides, and trehalose. Metabolism of any one compound is necessarily intertwined with other biosynthetic and degradative pathways within the cell, and necessary cofactors, intermediates, or substrates in one pathway are likely supplied or limited by another such pathway. Therefore, by modulating the activity of one or more of the proteins of the invention, the production or efficiency of activity of another fine chemical biosynthetic or degradative pathway may be impacted. For example, amino acids serve as the structural units of all proteins, yet may be present intracellularly in levels which are limiting for protein synthesis; therefore, by increasing the efficiency of production or the yields of one or more amino acids within the cell, proteins, such as biosynthetic or degradative proteins, may be more readily synthesized. Likewise, an alteration in a metabolic pathway enzyme such that a particular side reaction becomes more or less favored may result in the over- or under-production of one or more compounds which are utilized as intermediates or substrates for the production of a desired fine chemical.

The isolated nucleic acid sequences of the invention are contained within the genome of a Corynebacterium glutamicum strain available through the American Type Culture Collection, given designation ATCC 13032. The nucleotide sequence of the isolated C. glutamicum MP DNAs and the predicted amino acid sequences of the C. glutamicum MP proteins are shown in the Sequence Listing as odd-numbered SEQ ID NOs and even-numbered SEQ ID NOs, respectively. Computational analyses were performed which classified and/or identified these nucleotide sequences as sequences which encode metabolic pathway proteins.

The present invention also pertains to proteins which have an amino acid sequence which is substantially homologous to an amino acid sequence of the invention (e.g., the sequence of an even-numbered SEQ ID NO of the Sequence Listing). As used herein, a protein which has an amino acid sequence which is substantially homologous to a selected amino acid sequence is least about 50% homologous to the selected amino acid sequence, e.g., the entire selected amino acid sequence. A protein which has an amino acid sequence which is substantially homologous to a selected amino acid sequence can also be least about 50-60%, preferably at least about 60-70%, and more preferably at least about 70-80%, 80-90%, or 90-95%, and most preferably at least about 96%, 97%, 98%, 99% or more homologous to the selected amino acid sequence.

The MP protein or a biologically active portion or fragment thereof of the invention can catalyze an enzymatic reaction in one or more amino acid, vitamin, cofactor, nutraceutical, nucleotide, nucleoside, or trehalose metabolic pathways, or have one or more of the activities set forth in Table 1.

Various aspects of the invention are described in further detail in the following subsections:

A. Isolated Nucleic Acid Molecules

One aspect of the invention pertains to isolated nucleic acid molecules that encode MP polypeptides or biologically active portions thereof, as well as nucleic acid fragments sufficient for use as hybridization probes or primers for the identification or amplification of MP-encoding nucleic acid (e.g., MP DNA). As used herein, the term “nucleic acid molecule” is intended to include DNA molecules (e.g., cDNA or genomic DNA) and RNA molecules (e.g., mRNA) and analogs of the DNA or RNA generated using nucleotide analogs. This term also encompasses untranslated sequence located at both the 3′ and 5′ ends of the coding region of the gene: at least about 100 nucleotides of sequence upstream from the 5′ end of the coding region and at least about 20 nucleotides of sequence downstream from the 3′end of the coding region of the gene. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated MP nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g, a C. glutamicum cell). Moreover, an “isolated” nucleic acid molecule, such as a DNA molecule, can be substantially free of other cellular material, or culture medium when produced by recombinant techniques, or chemical precursors or other chemicals when chemically synthesized.

A nucleic acid molecule of the present invention, e.g., a nucleic acid molecule having a nucleotide sequence of an odd-numbered SEQ ID NO of the Sequence Listing, or a portion thereof, can be isolated using standard molecular biology techniques and the sequence information provided herein. For example, a C. glutamicum MP DNA can be isolated from a C. glutamicum library using all or portion of one of the odd-numbered SEQ ID NO sequences of the Sequence Listing as a hybridization probe and standard hybridization techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Moreover, a nucleic acid molecule encompassing all or a portion of one of the nucleic acid sequences of the invention (e.g., an odd-numbered SEQ ID NO:) can be isolated by the polymerase chain reaction using oligonucleotide primers designed based upon this sequence (e.g., a nucleic acid molecule encompassing all or a portion of one of the nucleic acid sequences of the invention (e.g., an odd-numbered SEQ ID NO of the Sequence Listing) can be isolated by the polymerase chain reaction using oligonucleotide primers designed based upon this same sequence). For example, mRNA can be isolated from normal endothelial cells (e.g., by the guanidinium-thiocyanate extraction procedure of Chirgwin et al. (1979) Biochemistry 18: 5294-5299) and DNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for polymerase chain reaction amplification can be designed based upon one of the nucleotide sequences shown in the Sequence Listing. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to an MP nucleotide sequence can be prepared by standard synthetic techniques, e.g., using an automated DNA synthesizer.

In a preferred embodiment, an isolated nucleic acid molecule of the invention comprises one of the nucleotide sequences shown in the Sequence Listing. The nucleic acid sequences of the invention, as set forth in the Sequence Listing, correspond to the Corynebacterium glutamicum MP DNAs of the invention. This DNA comprises sequences encoding MP proteins (i.e., the “coding region”, indicated in each odd-numbered SEQ ID NO: sequence in the Sequence Listing), as well as 5′ untranslated sequences and 3′ untranslated sequences, also indicated in each odd-numbered SEQ ID NO: in the Sequence Listing. Alternatively, the nucleic acid molecule can comprise only the coding region of any of the nucleic acid sequences of the Sequence Listing.

In another preferred embodiment, an isolated nucleic acid molecule of the invention comprises a nucleic acid molecule which is a complement of one of the nucleotide sequences of the invention (e.g., a sequence of an odd-numbered SEQ ID NO: of the Sequence Listing), or a portion thereof. A nucleic acid molecule which is complementary to one of the nucleotide sequences of the invention is one which is sufficiently complementary to one of the nucleotide sequences shown in the Sequence Listing (e.g., the sequence of an odd-numbered SEQ ID NO:) such that it can hybridize to one of the nucleotide sequences of the invention, thereby forming a stable duplex.

In still another preferred embodiment, an isolated nucleic acid molecule of the invention comprises a nucleotide sequence which is at least about 63%, 64%, 65%, 66%, 67%, 68%, 69%, or 70%%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more homologous to a nucleotide sequence of the invention (e.g., a sequence of an odd-numbered SEQ ID NO: of the Sequence Listing), or a portion thereof. Ranges and identity values intermediate to the above-recited ranges, (e.g., 70-90% identical or 80-95% identical) are also intended to be encompassed by the present invention. For example, ranges of identity values using a combination of any of the above values recited as upper and/or lower limits are intended to be included. In an additional preferred embodiment, an isolated nucleic acid molecule of the invention comprises a nucleotide sequence which hybridizes, e.g., hybridizes under stringent conditions, to one of the nucleotide sequences of the invention, or a portion thereof.

Moreover, the nucleic acid molecule of the invention can comprise only a portion of the coding region of the sequence of one of the odd-numbered SEQ ID NOs of the Sequence Listing, for example a fragment which can be used as a probe or primer or a fragment encoding a biologically active portion of an MP protein. The nucleotide sequences determined from the cloning of the MP genes from C. glutamicum allows for the generation of probes and primers designed for use in identifying and/or cloning MP homologues in other cell types and organisms, as well as MP homologues from other Corynebacteria or related species. The probe/primer typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of a sense strand of one of the nucleotide sequences of the invention (e.g., a sequence of one of the odd-numbered SEQ ID NOs of the Sequence Listing), an anti-sense sequence of one of these sequences, or naturally occurring mutants thereof. Primers based on a nucleotide sequence of the invention can be used in PCR reactions to clone MP homologues. Probes based on the MP nucleotide sequences can be used to detect transcripts or genomic sequences encoding the same or homologous proteins. In preferred embodiments, the probe further comprises a label group attached thereto, e.g. the label group can be a radioisotope, a fluorescent compound, an enzyme, or an enzyme co-factor. Such probes can be used as a part of a diagnostic test kit for identifying cells which misexpress an MP protein, such as by measuring a level of an MP-encoding nucleic acid in a sample of cells from a subject e.g., detecting MP mRNA levels or determining whether a genomic MP gene has been mutated or deleted.

In one embodiment, the nucleic acid molecule of the invention encodes a protein or portion thereof which includes an amino acid sequence which is sufficiently homologous to an amino acid sequence of the invention (e.g., a sequence of an even-numbered SEQ ID NO of the Sequence Listing) such that the protein or portion thereof maintains the ability to catalyze an enzymatic reaction in an amino acid, vitamin, cofactor, nutraceutical, nucleotide, nucleoside, or trehalose metabolic pathway. As used herein, the language “sufficiently homologous” refers to proteins or portions thereof which have amino acid sequences which include a minimum number of identical or equivalent (e.g., an amino acid residue which has a similar side chain as an amino acid residue in a sequence of one of the even-numbered SEQ ID NOs of the Sequence Listing) amino acid residues to an amino acid sequence of the invention such that the protein or portion thereof is able to catalyze an enzymatic reaction in a C. glutamicum amino acid, vitamin, cofactor, nutraceutical, nucleotide, nucleoside or trehalose metabolic pathway. Protein members of such metabolic pathways, as described herein, function to catalyze the biosynthesis or degradation of one or more of: amino acids, vitamins, cofactors, nutraceuticals, nucleotides, nucleosides, or trehalose. Examples of such activities are also described herein. Thus, “the function of an MP protein” contributes to the overall functioning of one or more such metabolic pathway and contributes, either directly or indirectly, to the yield, production, and/or efficiency of production of one or more fine chemicals. Examples of MP protein activities are set forth in Table 1.

In another embodiment, the protein is at least about 50-60%, preferably at least about 60-70%, and more preferably at least about 70-80%, 80-90%, 90-95%, and most preferably at least about 96%, 97%, 98%, 99% or more homologous to an entire amino acid sequence of the invention (e.g., a sequence of an even-numbered SEQ ID NO: of the Sequence Listing).

Portions of proteins encoded by the MP nucleic acid molecules of the invention are preferably biologically active portions of one of the MP proteins. As used herein, the term “biologically active portion of an MP protein” is intended to include a portion, e.g., a domain/motif, of an MP protein that catalyzes an enzymatic reaction in one or more C. glutamicum amino acid, vitamin, cofactor, nutraceutical, nucleotide, nucleoside, or trehalose metabolic pathways, or has an activity as set forth in Table 1. To determine whether an MP protein or a biologically active portion thereof can catalyze an enzymatic reaction in an amino acid, vitamin, cofactor, nutraceutical, nucleotide, nucleoside, or trehalose metabolic pathway, an assay of enzymatic activity may be performed. Such assay methods are well known to those of ordinary skill in the art, as detailed in Example 8 of the Exemplification.

Additional nucleic acid fragments encoding biologically active portions of an MP protein can be prepared by isolating a portion of one of the amino acid sequences of the invention (e.g., a sequence of an even-numbered SEQ ID NO: of the Sequence Listing), expressing the encoded portion of the MP protein or peptide (e.g., by recombinant expression in vitro) and assessing the activity of the encoded portion of the MP protein or peptide.

The invention further encompasses nucleic acid molecules that differ from one of the nucleotide sequences of the invention (e.g., a sequence of an odd-numbered SEQ ID NO: of the Sequence Listing) (and portions thereof) due to degeneracy of the genetic code and thus encode the same MP protein as that encoded by the nucleotide sequences of the invention. In another embodiment, an isolated nucleic acid molecule of the invention has a nucleotide sequence encoding a protein having an amino acid sequence shown in the Sequence Listing (e.g., an even-numbered SEQ ID NO:). In a still further embodiment, the nucleic acid molecule of the invention encodes a full length C. glutamicum protein which is substantially homologous to an amino acid sequence of the invention (encoded by an open reading frame shown in an odd-numbered SEQ ID NO: of the Sequence Listing).

It will be understood by one of ordinary skill in the art that in one embodiment the sequences of the invention are not meant to include the sequences of the prior art, such as those Genbank sequences set forth in Table 3 which were available prior to the present invention. In one embodiment, the invention includes nucleotide and amino acid sequences having a percent identity to a nucleotide or amino acid sequence of the invention which is greater than that of a sequence of the prior art, i.e the invention includes a nucleotide sequence which encodes a proteine sequence which is greater than and/or at least 71% identical to the proteine sequence designated SEQ ID NO:2 and/or a nucleotide sequence which encodes a proteine sequence which is greater than and/or at least 63% identical to the proteine sequence designated SEQ ID NO: 4.

TABLE 3
Alignment results

| Gene name | | | |
|---|---|---|---|
| (identifier) | Genbank hit | Homology | Reference |
| metH | GB_BA2:MTCY261 | 70.3% | Cole et al. (1998) |
| | Mycobacterium | | Nature 393, |
| | tuberculosis H37Rv | | 537-544 |
| | Complete genome | | |
| treS | GB_BA2:MTCY261 | 62.4% | Cole et al. (1998) |
| | Mycobacterium | | Nature 393, |
| | tuberculosis H37Rv | | 537-544 |
| | complete genome | | |

Homology: CLUSTAL-calculated percent identity (Open reading frames from the genome, translated into amino acid sequence)

One of ordinary skill in the art would be able to calculate the lower threshold of percent identity for any given sequence of the invention by examining the CLUSTAL-calculated percent identity scores set forth in Table 3 for each of the three top hits for the given sequence. One of ordinary skill in the art will also appreciate that nucleic acid and amino acid sequences having percent identities greater than the lower threshold so calculated (e.g., preferably at least about 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more identical) are also encompassed by the invention.

In addition to the C. glutamicum MP nucleotide sequences set forth in the Sequence Listing as odd-numbered SEQ ID NOs, it will be appreciated by one of ordinary skill in the art that DNA sequence polymorphisms that lead to changes in the amino acid sequences of MP proteins may exist within a population (e.g., the C. glutamicum population). Such genetic polymorphism in the MP gene may exist among individuals within a population due to natural variation. As used herein, the terms “gene” and “recombinant gene” refer to nucleic acid molecules comprising an open reading frame encoding an MP protein, preferably a C. glutamicum MP protein. Such natural variations can typically result in 1-5% variance in the nucleotide sequence of the MP gene. Any and all such nucleotide variations and resulting amino acid polymorphisms in MP that are the result of natural variation and that do not alter the functional activity of MP proteins are intended to be within the scope of the invention.

Nucleic acid molecules corresponding to natural variants and non-C. glutamicum homologues of the C. glutamicum MP DNA of the invention can be isolated based on their homology to the C. glutamicum MP nucleic acid disclosed herein using the C. glutamicum DNA, or a portion thereof, as a hybridization probe according to standard hybridization techniques under stringent hybridization conditions. Accordingly, in another embodiment, an isolated nucleic acid molecule of the invention is at least 15 nucleotides in length and hybridizes under stringent conditions to the nucleic acid molecule comprising a nucleotide sequence of an odd-numbered SEQ ID NO: of the Sequence Listing. In other embodiments, the nucleic acid is at least 30, 50, 100, 250 or more nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other. Such stringent conditions are known to one of ordinary skill in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to a nucleotide sequence of the invention corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein). In one embodiment, the nucleic acid encodes a natural C. glutamicum MP protein.

In addition to naturally-occurring variants of the MP sequence that may exist in the population, one of ordinary skill in the art will further appreciate that changes can be introduced by mutation into a nucleotide sequence of the invention, thereby leading to changes in the amino acid sequence of the encoded MP protein, without altering the functional ability of the MP protein. For example, nucleotide substitutions leading to amino acid substitutions at “non-essential” amino acid residues can be made in a nucleotide sequence of the invention. A “non-essential” amino acid residue is a residue that can be altered from the wild-type sequence of one of the MP proteins (e.g., an even-numbered SEQ ID NO: of the Sequence Listing) without altering the activity of said MP protein, whereas an “essential” amino acid residue is required for MP protein activity. Other amino acid residues, however, (e.g., those that are not conserved or only semi-conserved in the domain having MP activity) may not be essential for activity and thus are likely to be amenable to alteration without altering MP activity.

Accordingly, another aspect of the invention pertains to nucleic acid molecules encoding MP proteins that contain changes in amino acid residues that are not essential for MP activity. Such MP proteins differ in amino acid sequence from a sequence of an even-numbered SEQ ID NO: of the Sequence Listing yet retain at least one of the MP activities described herein. In one embodiment, the isolated nucleic acid molecule comprises a nucleotide sequence encoding a protein, wherein the protein comprises an amino acid sequence at least about 50% homologous to an amino acid sequence of the invention and is capable of catalyzing an enzymatic reaction in an amino acid, vitamin, cofactor, nutraceutical, nucleotide, nucleoside, or trehalose metabolic pathway, or has one or more activities set forth in Table 1. Preferably, the protein encoded by the nucleic acid molecule is at least about 50-60% homologous to the amino acid sequence of one of the odd-numbered SEQ ID NOs of the Sequence Listing, more preferably at least about 60-70% homologous to one of these sequences, even more preferably at least about 70-80%, 80-90%, 90-95% homologous to one of these sequences, and most preferably at least about 96%, 97%, 98%, or 99% homologous to one of the amino acid sequences of the invention.

To determine the percent homology of two amino acid sequences (e.g., one of the amino acid sequences of the invention and a mutant form thereof) or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence (e.g., one of the amino acid sequences of the invention) is occupied by the same amino acid residue or nucleotide as the corresponding position in the other sequence (e.g., a mutant form of the amino acid sequence), then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100).

An isolated nucleic acid molecule encoding an MP protein homologous to a protein sequence of the invention (e.g., a sequence of an even-numbered SEQ ID NO: of the Sequence Listing) can be created by introducing one or more nucleotide substitutions, additions or deletions into a nucleotide sequence of the invention such that one or more amino acid substitutions, additions or deletions are introduced into the encoded protein. Mutations can be introduced into one of the nucleotide sequences of the invention by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an MP protein is preferably replaced with another amino acid residue from the same side chain family. Alternatively, in another embodiment, mutations can be introduced randomly along all or part of an MP coding sequence, such as by saturation mutagenesis, and the resultant mutants can be screened for an MP activity described herein to identify mutants that retain MP activity. Following mutagenesis of the nucleotide sequence of one of the odd-numbered SEQ ID NOs of the Sequence Listing, the encoded protein can be expressed recombinantly and the activity of the protein can be determined using, for example, assays described herein (see Example 8 of the Exemplification).

In addition to the nucleic acid molecules encoding MP proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded DNA molecule or complementary to an mRNA sequence. Accordingly, an antisense nucleic acid can hydrogen bond to a sense nucleic acid. The antisense nucleic acid can be complementary to an entire MP coding strand, or to only a portion thereof. In one embodiment, an antisense nucleic acid molecule is antisense to a “coding region” of the coding strand of a nucleotide sequence encoding an MP protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding MP. The term “noncoding region” refers to 5′ and 3′ sequences which flank the coding region that are not translated into amino acids (i.e., also referred to as 5′ and 3′ untranslated regions).

Given the coding strand sequences encoding MP disclosed herein (e.g., the sequences set forth as odd-numbered SEQ ID NOs in the Sequence Listing), antisense nucleic acids of the invention can be designed according to the rules of Watson and Crick base pairing. The antisense nucleic acid molecule can be complementary to the entire coding region of MP mRNA, but more preferably is an oligonucleotide which is antisense to only a portion of the coding or noncoding region of MP mRNA. For example, the antisense oligonucleotide can be complementary to the region surrounding the translation start site of MP mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a cell or generated in situ such that they hybridize with or bind to cellular mRNA and/or genomic DNA encoding an MP protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. The antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong prokaryotic, viral, or eukaryotic promoter are preferred.

In yet another embodiment, the antisense nucleic acid molecule of the invention is an α-anomeric nucleic acid molecule. An anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave MP mRNA transcripts to thereby inhibit translation of MP mRNA. A ribozyme having specificity for an MP-encoding nucleic acid can be designed based upon the nucleotide sequence of an MP DNA disclosed herein (i.e., SEQ ID NO: 1 (RXA02229). For example, a derivative of a Tetrahymena L-19 IVS RNA can be constructed in which the nucleotide sequence of the active site is complementary to the nucleotide sequence to be cleaved in an MP-encoding mRNA. See, e.g., Cech et al. U.S. Pat. No. 4,987,071 and Cech et al. U.S. Pat. No. 5,116,742. Alternatively, MP mRNA can be used to select a catalytic RNA having a specific ribonuclease activity from a pool of RNA molecules. See, e.g., Bartel, D. and Szostak, J. W. (1993) Science 261:1411-1418.

Alternatively, MP gene expression can be inhibited by targeting nucleotide sequences complementary to the regulatory region of an MP nucleotide sequence (e.g., an MP promoter and/or enhancers) to form triple helical structures that prevent transcription of an MP gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

Another aspect of the invention pertains combinations of gene in the methionine and/or lysine metabolism. Preferred combinations are the combination of metZ with metC, metB (encoding Cystathionine-Synthase), metA (encoding homoserine-o-acetyltransferase), metE (encoding Methionine Synthase), metH (encoding Methionine Synthase, herein designated as SEQ ID No: 1), hom (encoding homoserine dehydrogenase), asd (encoding aspartatesemialdehyd dehydrogenase), ask (encoding aspartokinase) and rxa00657 (table 4).

TABLE 4
Genes in the Application

| Nucleic Acid | Amino Acid | Gene Function | |
|---|---|---|---|
| SEQ ID NO | SEQ ID NO | (identifier) | Function |
| 5 | 6 | MetZ | Acetylhomoserine |
| | | | sulfhydrolase |
| 7 | 8 | RXA00657 | |

It may be that all of the genes are expressed in a host strain. But it is also possible that only a part of the mentioned genes is chosen, e.g. metZ and metA, or metZ, metA, metH and hom or any other of the possible combinations. The genes may be altered in their nucleotide and in the corresponding amino acid sequence resulting in derivatives in such a way that their activity is altered under physiological conditions which leads to an increase in productivity and/or yield of a desired fine chemical. One class of such alterations or derivatives is well known for the nucleotide sequence of the ask gene encoding aspartokinase. These alterations lead to removal of feed back inhibition by the amino acids lysine and threonine and subsequently to lysine overproduction. In a preferred embodiment the metH gene or altered forms of the metH gene are used in a Corynebacterium strain in combination with ask, hom, metA and metZ or derivatives of these genes. In another preferred embodiment metH or altered forms of the metH gene are used in a Corynebacterium strain in combination with ask, hom, metA, metZ and metE or derivatives of these genes. In a more preferred embodiment the gene combinations metH or altered forms of the metH gene are combined with ask, hom, metA and metZ or derivatives of these genes, or metH is combined with ask, hom, metA, metZ and metE or derivatives of these genes in a Corynebacterium strain and sulfur sources like sulfates, thiosulfates, sulfites and also more reduced sulfur sources like H₂S and sulfides and derivatives are used in the growth medium. Also sulfur sources like methyl mercaptan, methanesulfonic acid, thioglycolates, thiocyanates, thiourea, sulfur containing amino acids like cysteine and other sulfur containing compounds can be fed. Another aspect of the invention pertains to the use of the above mentioned gene combinations in a Corynebacterium strain wich is before or after introduction of the genes mutagenized by radiation or by well known mutagenic chemicals and selected for resistancy against high concentrations of the fine chmical of interest, e.g. lysine or methionine or anologes of the desired fine chemical like the methionine analogons ethionine or methyl methionine or others. In another embodiment the gene combinations mentioned above can be expressed in a Corynebacterium strain having particular gene disruptions. Preferred are gene diruptions that encode proteins that favor carbon flux to undesired metabolites. In case methionine is the desired fine chemical the formation of lysine may be unfavorable. In such a case the combination of the above mentioned genes should proceed in a Corynebacterium strain bearing a gene disruption of the lysA gene (encoding diaminopimelate decarboxylase) or the ddh gene (encoding the meso-diaminopimelate dehydrogenase catalysing the conversion of tetrahydropicolinate to meso-diaominopimelate). In a preferred embodiment a favorable combination of the above mentioned genes are all altered in such a way that their gene products are not feed back inhibited by endproducts or metabolites of the biosynthetic pathway leading to the desired fine chemical. In the case that the desired fine chemical is methionine, the gene combinations may be expressed in a strain previously treated with mutagenic agents or radiation and selected for the above mentioned resistancies. Additionally the strain should be grown in a growth medium containing one or ore of the above mentioned sulfur sources.

Another aspect of the invention pertains combinations of genes involved in the metabolism of trehalose and the combination of genes involved in the metabolism of trehalose and other mono-, oligo- or polymeric saccharides. Preferred are combinations of the gene for trehalose synthase (herein designated as SEQ ID No: 3) with genes disclosed in table 5.

Another aspect of the invention is the combination of the gene for trehalose synthase with genes involved in saccharide import, as e.g. the genes for the PTS system (as disclosed in table 5), other saccharide transport systems or proteins facilitating saccharide efflux from the cell into the surrounding environment.

TABLE 5
PHOSPHOENOLPYRUVATE: SUGAR PHOSPHOTRANSFERASE SYSTEM

| | Amino | | |
|---|---|---|---|
| Nucleotide | Acid | | |
| SEQ | SEQ | Identification | |
| ID NO | ID NO | code | Function |
| 9 | 10 | RXS00315 | PTS SYSTEM, SUCROSE-SPECIFIC IIABC COMPONENT (EIIABC |
| | | | SCR) (SUCROSE-PERMEASE IIABC COMPONENT(PHOSPHO- |
| | | | TRANSFERASE ENZYME II, ABC COMPONENT) (EC 2.7.1.69) |
| 11 | 12 | RXN01299 | PTS SYSTEM, FRUCTOSE-SPECIFIC IIBC COMPONENT (EC |
| | | | 2.7.1.69) |
| 13 | 14 | RXA00951 | PTS SYSTEM, MANNITOL (CRYPTIC)-SPECIFIC IIA COMPO- |
| | | | NENT (EIIA-(C)MTL) (MANNITOL (CRYPTIC)-PERMEASE IIA |
| | | | COMPONENT) (PHOSPHOTRANSFERASE ENZYME II, A COM- |
| | | | PONENT) (EC 2.7.1.69) |
| 15 | 16 | RXN01244 | PHOSPHOENOLPYRUVATE-PROTEIN PHOSPHOTRANSFE- |
| | | | RASE (EC 2.7.3.9) |
| 17 | 18 | RXA01300 | PHOSPHOCARRIERPROTEIN HPR |
| 19 | 20 | RXN03002 | PTS SYSTEM, MANNITOL (CRYPTIC)-SPECIFIC IIA COMPO- |
| | | | NENT (EHA-(C)MTL) (MANNITOL (CRYPTIC)-PERMEASE IIA |
| | | | COMPONENT) (PHOSPHOTRANSFERASE ENZYME II, A COM- |
| | | | PONENT) (EC 2.7.1.69) |
| 21 | 22 | RXC00953 | Membrane Spanning Protein involved in PTS system |
| 23 | 24 | RXC03001 | Membrane Spanning Protein involved in PTS system |
| 25 | 26 | RXN01943 | PTS SYSTEM, GLUCOSE-SPECIFIC IIABC COMPONENT (EC |
| | | | 2.7.1.69) |
| 27 | 28 | RXA01503 | PTS SYSTEM, BETA-GLUCOSIDES-SPECIFIC IIABC COMPO- |
| | | | NENT (EHABC-BGL) (BETA-GLUCOSIDES-PERMEASE IIABC |
| | | | COMPONENT) (PHOSPHOTRANSFERASE ENZYME II, ABC |
| | | | COMPONENT) (EC 2.7.1.69) |

Trehalose

| Nucleic | Amino | | |
|---|---|---|---|
| Acid | Acid | | |
| SEQ | SEQ | Identification | |
| ID NO | ID NO | code | Function |
| 29 | 30 | RXN00351 | ALPHA,ALPHA-TREHALOSE-PHOSPHATE SYNTHASE |
| | | | (UDP-FORMING) 56 KD SUBUNIT (EC 2.4.1.15) |
| 31 | 32 | RXA00347 | TREHALOSE-PHOSPHATASE (EC 3.1.3.12) |
| 33 | 34 | RXN01239 | maltooligosyltrehalose synthase |
| 35 | 36 | RXA02645 | maltooligosyltrehalose trehalohydrolase |
| 37 | 38 | RXN02355 | TREHALOSE/MALTOSE BINDING PROTEIN |
| 39 | 40 | RXN02909 | Hypothetical Trehalose-Binding Protein |
| 41 | 42 | RX500349 | Hypothetical Trehalose Transport Protein |
| 43 | 44 | RXS03183 | TREHALOSE/MALTOSE BINDING PROTEIN |
| 45 | 46 | RXC00874 | transmebrane protein involved in trehalose metabolism |

Another aspect of the invention pertains to the use of the above mentioned gene combinations in a Corynebacterium strain wich is before or after introduction of the genes mutagenized by radiation or by well known mutagenic chemicals and selected for resistancy against high concentrations of feedstock (as e.g. glucose or other saccharides) or the fine chemical of interest, e.g. trehalose or other saccharides.

In another embodiment the gene combinations mentioned above can be expressed in a Corynebacterium strain having particular gene disruptions or gene attenuations (i.e. genes which biological activity is reduced compared to the normal level). Preferred are disruptions or attenuations of genes that encode proteins that favor carbon flux to metabolic pathways which do not lead to the desired fine chemical. In case of trehalose being the desired fine chemical, such less desired metabolic pathways may be e.g. glycolysis or pentose phosphate cycle.

B. Recombinant Expression Vectors and Host Cells

Another aspect of the invention pertains to vectors, preferably expression vectors, containing a nucleic acid encoding an MP protein (or a portion thereof) or combinations of genes wherein at least one gene encodes for an MP protein. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, repressor binding sites, activator binding sites, enhancers and other expression control elements (e.g., terminators, polyadenylation signals, or other elements of mRNA secondary structure). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells. Preferred regulatory sequences are, for example, promoters such as cos-, tac-, trp-, tet-, trp-tet-, lpp-, lac-, lpp-lac-, laciq-, T7-, T5-, T3-, gal-, trc-, ara-, SP6-, arny, SPO₂, ë-P_{R}- or ë P_{L}, which are used preferably in bacteria. Additional regulatory sequences are, for example, promoters from yeasts and fungi, such as ADCl, MFá, AC, P-60, CYC1, GAPDH, TEF, rp28, ADH, promoters from plants such as CaMV/35S, SSU, OCS, lib4, usp, STLS1, B33, nos or ubiquitin- or phaseolin-promoters. It is also possible to use artificial promoters. It will be appreciated by one of ordinary skill in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., MP proteins, mutant forms of MP proteins, fusion proteins, etc.).

The recombinant expression vectors of the invention can be designed for expression of MP proteins in prokaryotic or eukaryotic cells. For example, MP genes can be expressed in bacterial cells such as C. glutamicum, insect cells (using baculovirus expression vectors), yeast and other fungal cells (see Romanos, M. A. et al. (1992) “Foreign gene expression in yeast: a review”, Yeast 8: 423-488; van den Hondel, C. A. M. J. J. et al. (1991) “Heterologous gene expression in filamentous fungi” in: More Gene Manipulations in Fungi, J. W. Bennet & L. L. Lasure, eds., p. 396-428: Academic Press: San Diego; and van den Hondel, C. A. M. J. J. & Punt, P. J. (1991) “Gene transfer systems and vector development for filamentous fungi, in: Applied Molecular Genetics of Fungi, Peberdy, J. F. et al., eds., p. 1-28, Cambridge University Press: Cambridge), algae and multicellular plant cells (see Schmidt, R. and Willmitzer, L. (1988) High efficiency Agrobacterium tumefaciens-mediated transformation of Arabidopsis thaliana leaf and cotyledon explants” Plant Cell Rep.: 583-586), or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out with vectors containing constitutive or inducible promoters directing the expression of either fusion or non-fusion proteins. Fusion vectors add a number of amino acids to a protein encoded therein, usually to the amino terminus of the recombinant protein but also to the C-terminus or fused within suitable regions in the proteins. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include PGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. In one embodiment, the coding sequence of the MP protein is cloned into a PGEX expression vector to create a vector encoding a fusion protein comprising, from the N-terminus to the C-terminus, GST-thrombin cleavage site-X protein. The fusion protein can be purified by affinity chromatography using glutathione-agarose resin. Recombinant MP protein unfused to GST can be recovered by cleavage of the fusion protein with thrombin.

Examples of suitable inducible non-fusion E. coli expression vectors include pTrc (Amann et al., (1988) Gene 69:301-315) pLG338, pACYC184, pBR322, pUC18, pUC19, pKC30, pRep4, pHS1, pHS2, pPLc236, pMBL24, pLG200, pUR290, pIN-III113-B1, ëgt11, pBdCl, and pET 11d (Studier et al., Gene Expression Technology: Methods in Enzymology 185, Academic Press, San Diego, Calif. (1990) 60-89; and Pouwels et al., eds. (1985) Cloning Vectors. Elsevier: New York IBSN 0 444 904018). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter. For transformation of other varieties of bacteria, appropriate vectors may be selected. For example, the plasmids pIJ101, pIJ364, pIJ702 and pIJ361 are known to be useful in transforming Streptomyces, while plasmids pUB110, pC194, or pBD214 are suited for transformation of Bacillus species. Several plasmids of use in the transfer of genetic information into Corynebacterium include pHM1519, pBL1, pSA77, or pAJ667 (Pouwels et al., eds. (1985) Cloning Vectors. Elsevier: New York IBSN 0 444 904018).

One strategy to maximize recombinant protein expression is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in the bacterium chosen for expression, such as C. glutamicum (Wada et al. (1992) Nucleic Acids Res. 20:2111-2118). Such alteration of nucleic acid sequences of the invention can be carried out by standard DNA synthesis techniques.

In another embodiment, the MP protein expression vector is a yeast expression vector. Examples of vectors for expression in yeast S. cerevisiae include pYepSec1 (Baldari, et al., (1987) Embo J. 6:229-234), , 2 i, pAG-1, Yep6, Yep13, pEMBLYe23, pMFa (Kurjan and Herskowitz, (1982) Cell 30:933-943), pJRY88 (Schultz et al., (1987) Gene 54:113-123), and pYES2 (Invitrogen Corporation, San Diego, Calif.). Vectors and methods for the construction of vectors appropriate for use in other fungi, such as the filamentous fungi, include those detailed in: van den Hondel, C. A. M. J. J. & Punt, P. J. (1991) “Gene transfer systems and vector development for filamentous fungi, in: Applied Molecular Genetics of Fungi, J. F. Peberdy, et al., eds., p. 1-28, Cambridge University Press: Cambridge, and Pouwels et al., eds. (1985) Cloning Vectors. Elsevier: New York (IBSN 0 444 904018).

Alternatively, the MP proteins of the invention can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In another embodiment, the MP proteins of the invention may be expressed in unicellular plant cells (such as algae) or in plant cells from higher plants (e.g., the spermatophytes, such as crop plants). Examples of plant expression vectors include those detailed in: Becker, D., Kemper, E., Schell, J. and Masterson, R. (1992) “New plant binary vectors with selectable markers located proximal to the left border”, Plant Mol. Biol. 20: 1195-1197; and Bevan, M. W. (1984) “Binary Agrobacterium vectors for plant transformation”, Nucl. Acid. Res. 12: 8711-8721, and include LGV23, pGHlac+, pBIN19, pAK2004, and pDH51 (Pouwels et al., eds. (1985) Cloning Vectors. Elsevier: New York IBSN 0 444 904018).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to MP mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an MP protein can be expressed in bacterial cells such as C. glutamicum, insect cells, yeast or mammalian cells (such as Chinese hamster ovary cells (CHO) or COS cells). Other suitable host cells are known to those of ordinary skill in the art. Microorganisms related to Corynebacterium glutamicum which may be conveniently used as host cells for the nucleic acid and protein molecules of the invention are set forth in Table 2.

Vector DNA can be introduced into prokaryotic or eukaryotic cells via conventional transformation or transfection techniques. As used herein, the terms “transformation” and “transfection”, “conjugation” and “transduction” are intended to refer to a variety of art-recognized techniques for introducing foreign nucleic acid (e.g., linear DNA or RNA (e.g., a linearized vector or a gene construct alone without a vector) or nucleic acid in the form of a vector (e.g., a plasmid, phage, phasmid, phagemid, transposon or other DNA) into a host cell, including calcium phosphate or calcium chloride co-precipitation, DEAE-dextran-mediated transfection, lipofection, natural competence, chemical-mediated transfer, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an MP protein or can be introduced on a separate vector. Cells stably transfected with the introduced nucleic acid can be identified by drug selection (e.g., cells that have incorporated the selectable marker gene will survive, while the other cells die).

To create a homologous recombinant microorganism, a vector is prepared which contains at least a portion of an MP gene into which a deletion, addition or substitution has been introduced to thereby alter, e.g., functionally disrupt, the MP gene.

Preferably, this MP gene is a Corynebacterium glutamicum MP gene, but it can be a homologue from a related bacterium or even from a mammalian, yeast, or insect source. In a preferred embodiment, the vector is designed such that, upon homologous recombination, the endogenous MP gene is functionally disrupted (i.e., no longer encodes a functional protein; also referred to as a “knock out” vector). Alternatively, the vector can be designed such that, upon homologous recombination, the endogenous MP gene is mutated or otherwise altered but still encodes functional protein (e.g., the upstream regulatory region can be altered to thereby alter the expression of the endogenous MP protein). In the homologous recombination vector, the altered portion of the MP gene is flanked at its 5′ and 3′ ends by additional nucleic acid of the MP gene to allow for homologous recombination to occur between the exogenous MP gene carried by the vector and an endogenous MP gene in a microorganism. The additional flanking MP nucleic acid is of sufficient length for successful homologous recombination with the endogenous gene. Typically, several kilobases of flanking DNA (both at the 5′ and 3′ ends) are included in the vector (see e.g., Thomas, K. R., and Capecchi, M. R. (1987) Cell 51: 503 for a description of homologous recombination vectors). The vector is introduced into a microorganism (e.g., by electroporation) and cells in which the introduced MP gene has homologously recombined with the endogenous MP gene are selected, using art-known techniques.

In another embodiment, recombinant microorganisms can be produced which contain selected systems which allow for regulated expression of the introduced gene. For example, inclusion of an MP gene on a vector placing it under control of the lac operon permits expression of the MP gene only in the presence of IPTG. Such regulatory systems are well known in the art.

In another embodiment, an endogenous MP gene in a host cell is disrupted (e.g., by homologous recombination or other genetic means known in the art) such that expression of its protein product does not occur. In another embodiment, an endogenous or introduced MP gene in a host cell has been altered by one or more point mutations, deletions, or inversions, but still encodes a functional MP protein. In still another embodiment, one or more of the regulatory regions (e.g., a promoter, repressor, or inducer) of an MP gene in a microorganism has been altered (e.g., by deletion, truncation, inversion, or point mutation) such that the expression of the MP gene is modulated. One of ordinary skill in the art will appreciate that host cells containing more than one of the described MP gene and protein modifications may be readily produced using the methods of the invention, and are meant to be included in the present invention.

A host cell of the invention, such as a prokaryotic or eukaryotic host cell in culture, can be used to produce (i.e., express) an MP protein. Accordingly, the invention further provides methods for producing MP proteins using the host cells of the invention. In one embodiment, the method comprises culturing the host cell of invention (into which a recombinant expression vector encoding an MP protein has been introduced, or into which genome has been introduced a gene encoding a wild-type or altered MP protein) in a suitable medium until MP protein is produced. In another embodiment, the method further comprises isolating MP proteins from the medium or the host cell.

C. Isolated MP Proteins

Another aspect of the invention pertains to isolated MP proteins, and biologically active portions thereof. An “isolated” or “purified” protein or biologically active portion thereof is substantially free of cellular material when produced by recombinant DNA techniques, or chemical precursors or other chemicals when chemically synthesized. The language “substantially free of cellular material” includes preparations of MP protein in which the protein is separated from cellular components of the cells in which it is naturally or recombinantly produced. In one embodiment, the language “substantially free of cellular material” includes preparations of MP protein having less than about 30% (by dry weight) of non-MP protein (also referred to herein as a “contaminating protein”), more preferably less than about 20% of non-MP protein, still more preferably less than about 10% of non-MP protein, and most preferably less than about 5% non-MP protein. When the MP protein or biologically active portion thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other chemicals” includes preparations of MP protein in which the protein is separated from chemical precursors or other chemicals which are involved in the synthesis of the protein. In one embodiment, the language “substantially free of chemical precursors or other chemicals” includes preparations of MP protein having less than about 30% (by dry weight) of chemical precursors or non-MP chemicals, more preferably less than about 20% chemical precursors or non-MP chemicals, still more preferably less than about 10% chemical precursors or non-MP chemicals, and most preferably less than about 5% chemical precursors or non-MP chemicals. In preferred embodiments, isolated proteins or biologically active portions thereof lack contaminating proteins from the same organism from which the MP protein is derived. Typically, such proteins are produced by recombinant expression of, for example, a C. glutamicum MP protein in a microorganism such as C. glutamicum.

An isolated MP protein or a portion thereof of the invention can catalyze an enzymatic reaction in an amino acid, vitamin, cofactor, nutraceutical, nucleotide, nucleoside, or trehalose metabolic pathway, or has one or more of the activities set forth in Table 1. In preferred embodiments, the protein or portion thereof comprises an amino acid sequence which is sufficiently homologous to an amino acid sequence of the invention (e.g., a sequence of an even-numbered SEQ ID NO: of the Sequence Listing) such that the protein or portion thereof maintains the ability to catalyze an enzymatic reaction in an amino acid, vitamin, cofactor, nutraceutical, nucleotide, nucleoside, or trehalose metabolic pathway. The portion of the protein is preferably a biologically active portion as described herein. In another preferred embodiment, an MP protein of the invention has an amino acid sequence set forth as an even-numbered SEQ ID NO: of the Sequence Listing. In yet another preferred embodiment, the MP protein has an amino acid sequence which is encoded by a nucleotide sequence which hybridizes, e.g., hybridizes under stringent conditions, to a nucleotide sequence of the invention (e.g., a sequence of an odd-numbered SEQ ID NO: of the Sequence Listing). In still another preferred embodiment, the MP protein has an amino acid sequence which is encoded by a nucleotide sequence that is preferably at least about 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more homologous to one of the nucleic acid sequences of the invention, or a portion thereof. Ranges and identity values intermediate to the above-recited values, (e.g., 70-90% identical or 80-95% identical) are also intended to be encompassed by the present invention. For example, ranges of identity values using a combination of any of the above values recited as upper and/or lower limits are intended to be included. The preferred MP proteins of the present invention also preferably possess at least one of the MP activities described herein. For example, a preferred MP protein of the present invention includes an amino acid sequence encoded by a nucleotide sequence which hybridizes, e.g., hybridizes under stringent conditions, to a nucleotide sequence of the invention, and which can catalyze an enzymatic reaction in an amino acid, vitamin, cofactor, nutraceutical, nucleotide, nucleoside, or trehalose metabolic pathway, or which has one or more of the activities set forth in Table 1.

In other embodiments, the MP protein is substantially homologous to an amino acid sequence of the invention (e.g., a sequence of an even-numbered SEQ ID NO: of the Sequence Listing) and retains the functional activity of the protein of one of the amino acid sequences of the invention yet differs in amino acid sequence due to natural variation or mutagenesis, as described in detail in subsection I above. Accordingly, in another embodiment, the MP protein is a protein which comprises an amino acid sequence which is preferably at least about 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more homologous to an entire amino acid sequence of the invention and which has at least one of the MP activities described herein. Ranges and identity values intermediate to the above-recited values, (e.g., 70-90% identical or 80-95% identical) are also intended to be encompassed by the present invention. For example, ranges of identity values using a combination of any of the above values recited as upper and/or lower limits are intended to be included. In another embodiment, the invention pertains to a full length C. glutamicum protein which is substantially homologous to an entire amino acid sequence of the invention.

Biologically active portions of an MP protein include peptides comprising amino acid sequences derived from the amino acid sequence of an MP protein, e.g., an amino acid sequence of an even-numbered SEQ ID NO: of the Sequence Listing or the amino acid sequence of a protein homologous to an MP protein, which include fewer amino acids than a full length MP protein or the full length protein which is homologous to an MP protein, and exhibit at least one activity of an MP protein. Typically, biologically active portions (peptides, e.g., peptides which are, for example, 5, 10, 15, 20, 30, 35, 36, 37, 38, 39, 40, 50, 100 or more amino acids in length) comprise a domain or motif with at least one activity of an MP protein. Moreover, other biologically active portions, in which other regions of the protein are deleted, can be prepared by recombinant techniques and evaluated for one or more of the activities described herein. Preferably, the biologically active portions of an MP protein include one or more selected domains/motifs or portions thereof having biological activity.

MP proteins are preferably produced by recombinant DNA techniques. For example, a nucleic acid molecule encoding the protein is cloned into an expression vector (as described above), the expression vector is introduced into a host cell (as described above) and the MP protein is expressed in the host cell. The MP protein can then be isolated from the cells by an appropriate purification scheme using standard protein purification techniques. Alternative to recombinant expression, an MP protein, polypeptide, or peptide can be synthesized chemically using standard peptide synthesis techniques. Moreover, native MP protein can be isolated from cells (e.g., endothelial cells), for example using an anti-MP antibody, which can be produced by standard techniques utilizing an MP protein or fragment thereof of this invention.

The invention also provides MP chimeric or fusion proteins. As used herein, an MP “chimeric protein” or “fusion protein” comprises an MP polypeptide operatively linked to a non-MP polypeptide. An “MP polypeptide” refers to a polypeptide having an amino acid sequence corresponding to MP, whereas a “non-MP polypeptide” refers to a polypeptide having an amino acid sequence corresponding to a protein which is not substantially homologous to the MP protein, e.g., a protein which is different from the MP protein and which is derived from the same or a different organism. Within the fusion protein, the term “operatively linked” is intended to indicate that the MP polypeptide and the non-MP polypeptide are fused in-frame to each other. The non-MP polypeptide can be fused to the N-terminus or C-terminus of the MP polypeptide. For example, in one embodiment the fusion protein is a GST-MP fusion protein in which the MP sequences are fused to the C-terminus of the GST sequences. Such fusion proteins can facilitate the purification of recombinant MP proteins. In another embodiment, the fusion protein is an MP protein containing a heterologous signal sequence at its N-terminus. In certain host cells (e.g., mammalian host cells), expression and/or secretion of an MP protein can be increased through use of a heterologous signal sequence.

Preferably, an MP chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An MP-encoding nucleic acid can be cloned into such an expression vector such that the fusion moiety is linked in-frame to the MP protein.

Homologues of the MP protein can be generated by mutagenesis, e.g., discrete point mutation or truncation of the MP protein. As used herein, the term “homologue” refers to a variant form of the MP protein which acts as an agonist or antagonist of the activity of the MP protein. An agonist of the MP protein can retain substantially the same, or a subset, of the biological activities of the MP protein. An antagonist of the MP protein can inhibit one or more of the activities of the naturally occurring form of the MP protein, by, for example, competitively binding to a downstream or upstream member of the MP cascade which includes the MP protein. Thus, the C. glutamicum MP protein and homologues thereof of the present invention may modulate the activity of one or more metabolic pathways in which MP proteins play a role in this microorganism.

In an alternative embodiment, homologues of the MP protein can be identified by screening combinatorial libraries of mutants, e.g., truncation mutants, of the MP protein for MP protein agonist or antagonist activity. In one embodiment, a variegated library of MP variants is generated by combinatorial mutagenesis at the nucleic acid level and is encoded by a variegated gene library. A variegated library of MP variants can be produced by, for example, enzymatically ligating a mixture of synthetic oligonucleotides into gene sequences such that a degenerate set of potential MP sequences is expressible as individual polypeptides, or alternatively, as a set of larger fusion proteins (e.g., for phage display) containing the set of MP sequences therein. There are a variety of methods which can be used to produce libraries of potential MP homologues from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential MP sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of the MP protein coding can be used to generate a variegated population of MP fragments for screening and subsequent selection of homologues of an MP protein. In one embodiment, a library of coding sequence fragments can be generated by treating a double stranded PCR fragment of an MP coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the MP protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of MP homologues. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify MP homologues (Arkin and Yourvan (1992) PNAS 89:7811-7815; Delgrave et al. (1993) Protein Engineering 6(3):327-331).

In another embodiment, cell based assays can be exploited to analyze a variegated MP library, using methods well known in the art.

D. Uses and Methods of the Invention

The nucleic acid molecules, proteins, protein homologues, fusion proteins, primers, vectors, and host cells described herein can be used in one or more of the following methods: identification of C. glutamicum and related organisms; mapping of genomes of organisms related to C. glutamicum; identification and localization of C. glutamicum sequences of interest; evolutionary studies; determination of MP protein regions required for function; modulation of an MP protein activity; modulation of the activity of an MP pathway; and modulation of cellular production of a desired compound, such as a fine chemical. The MP nucleic acid molecules of the invention have a variety of uses. First, they may be used to identify an organism as being Corynebacterium glutamicum or a close relative thereof. Also, they may be used to identify the presence of C. glutamicum or a relative thereof in a mixed population of microorganisms. The invention provides the nucleic acid sequences of a number of C. glutamicum genes; by probing the extracted genomic DNA of a culture of a unique or mixed population of microorganisms under stringent conditions with a probe spanning a region of a C. glutamicum gene which is unique to this organism, one can ascertain whether this organism is present. Although Corynebacterium glutamicum itself is not pathogenic to humans, it is related to species which are human pathogens, such as Corynebacterium diphtheriae. Corynebacterium diphtheriae is the causative agent of diphtheria, a rapidly developing, acute, febrile infection which involves both local and systemic pathology. In this disease, a local lesion develops in the upper respiratory tract and involves necrotic injury to epithelial cells; the bacilli secrete toxin which is disseminated through this lesion to distal susceptible tissues of the body. Degenerative changes brought about by the inhibition of protein synthesis in these tissues, which include heart, muscle, peripheral nerves, adrenals, kidneys, liver and spleen, result in the systemic pathology of the disease. Diphtheria continues to have high incidence in many parts of the world, including Africa, Asia, Eastern Europe and the independent states of the former Soviet Union. An ongoing epidemic of diphtheria in the latter two regions has resulted in at least 5,000 deaths since 1990.

In one embodiment, the invention provides a method of identifying the presence or activity of Cornyebacterium diphtheriae in a subject. This method includes detection of one or more of the nucleic acid or amino acid sequences of the invention (e.g., the sequences set forth as odd-numbered or even-numbered SEQ ID NOs, respectively, in the Sequence Listing) in a subject, thereby detecting the presence or activity of Corynebacterium diphtheriae in the subject. C. glutamicum and C. diphtheriae are related bacteria, and many of the nucleic acid and protein molecules in C. glutamicum are homologous to C. diphtheriae nucleic acid and protein molecules, and can therefore be used to detect C. diphtheriae in a subject.

The nucleic acid and protein molecules of the invention may also serve as markers for specific regions of the genome. This has utility not only in the mapping of the genome, but also for functional studies of C. glutamicum proteins. For example, to identify the region of the genome to which a particular C. glutamicum DNA-binding protein binds, the C. glutamicum genome could be digested, and the fragments incubated with the DNA-binding protein. Those which bind the protein may be additionally probed with the nucleic acid molecules of the invention, preferably with readily detectable labels; binding of such a nucleic acid molecule to the genome fragment enables the localization of the fragment to the genome map of C. glutamicum, and, when performed multiple times with different enzymes, facilitates a rapid determination of the nucleic acid sequence to which the protein binds. Further, the nucleic acid molecules of the invention may be sufficiently homologous to the sequences of related species such that these nucleic acid molecules may serve as markers for the construction of a genomic map in related bacteria, such as Brevibacterium lactofermentum.

The MP nucleic acid molecules of the invention are also useful for evolutionary and protein structural studies. The metabolic processes in which the molecules of the invention participate are utilized by a wide variety of prokaryotic and eukaryotic cells; by comparing the sequences of the nucleic acid molecules of the present invention to those encoding similar enzymes from other organisms, the evolutionary relatedness of the organisms can be assessed. Similarly, such a comparison permits an assessment of which regions of the sequence are conserved and which are not, which may aid in determining those regions of the protein which are essential for the functioning of the enzyme. This type of determination is of value for protein engineering studies and may give an indication of what the protein can tolerate in terms of mutagenesis without losing function.

Manipulation of the MP nucleic acid molecules of the invention may result in the production of MP proteins having functional differences from the wild-type MP proteins. These proteins may be improved in efficiency or activity, may be present in greater numbers in the cell than is usual, or may be decreased in efficiency or activity.

The invention also provides methods for screening molecules which modulate the activity of an MP protein, either by interacting with the protein itself or a substrate or binding partner of the MP protein, or by modulating the transcription or translation of an MP nucleic acid molecule of the invention. In such methods, a microorganism expressing one or more MP proteins of the invention is contacted with one or more test compounds, and the effect of each test compound on the activity or level of expression of the MP protein is assessed.

When the desired fine chemical to be isolated from large-scale fermentative culture of C. glutamicum is an amino acid, a vitamin, a cofactor, a nutraceutical, a nucleotide, a nucleoside, or trehalose, modulation of the activity or efficiency of activity of one or more of the proteins of the invention by recombinant genetic mechanisms may directly impact the production of one of these fine chemicals. For example, in the case of an enzyme in a biosynthetic pathway for a desired amino acid, improvement in efficiency or activity of the enzyme (including the presence of multiple copies of the gene) should lead to an increased production or efficiency of production of that desired amino acid. In the case of an enzyme in a biosynthetic pathway for an amino acid whose synthesis is in competition with the synthesis of a desired amino acid, any decrease in the efficiency or activity of this enzyme (including deletion of the gene) should result in an increase in production or efficiency of production of the desired amino acid, due to decreased competition for intermediate compounds and/or energy. In the case of an enzyme in a degradation pathway for a desired amino acid, any decrease in efficiency or activity of the enzyme should result in a greater yield or efficiency of production of the desired product due to a decrease in its degradation. Lastly, mutagenesis of an enzyme involved in the biosynthesis of a desired amino acid such that this enzyme is no longer is capable of feedback inhibition should result in increased yields or efficiency of production of the desired amino acid. The same should apply to the biosynthetic and degradative enzymes of the invention involved in the metabolism of vitamins, cofactors, nutraceuticals, nucleotides, nucleosides and trehalose.

Similarly, when the desired fine chemical is not one of the aforementioned compounds, the modulation of activity of one of the proteins of the invention may still impact the yield and/or efficiency of production of the compound from large-scale culture of C. glutamicum. The metabolic pathways of any organism are closely interconnected; the intermediate used by one pathway is often supplied by a different pathway. Enzyme expression and function may be regulated based on the cellular levels of a compound from a different metabolic process, and the cellular levels of molecules necessary for basic growth, such as amino acids and nucleotides, may critically affect the viability of the microorganism in large-scale culture. Thus, modulation of an amino acid biosynthesis enzyme, for example, such that it is no longer responsive to feedback inhibition or such that it is improved in efficiency or turnover may result in increased cellular levels of one or more amino acids. In turn, this increased pool of amino acids provides not only an increased supply of molecules necessary for protein synthesis, but also of molecules which are utilized as intermediates and precursors in a number of other biosynthetic pathways. If a particular amino acid had been limiting in the cell, its increased production might increase the ability of the cell to perform numerous other metabolic reactions, as well as enabling the cell to more efficiently produce proteins of all kinds, possibly increasing the overall growth rate or survival ability of the cell in large scale culture. Increased viability improves the number of cells capable of producing the desired fine chemical in fermentative culture, thereby increasing the yield of this compound. Similar processes are possible by the modulation of activity of a degradative enzyme of the invention such that the enzyme no longer catalyzes, or catalyzes less efficiently, the degradation of a cellular compound which is important for the biosynthesis of a desired compound, or which will enable the cell to grow and reproduce more efficiently in large-scale culture. It should be emphasized that optimizing the degradative activity or decreasing the biosynthetic activity of certain molecules of the invention may also have a beneficial effect on the production of certain fine chemicals from C. glutamicum. For example, by decreasing the efficiency of activity of a biosynthetic enzyme in a pathway which competes with the biosynthetic pathway of a desired compound for one or more intermediates, more of those intermediates should be available for conversion to the desired product. A similar situation may call for the improvement of degradative ability or efficiency of one or more proteins of the invention.

This aforementioned list of mutagenesis strategies for MP proteins to result in increased yields of a desired compound is not meant to be limiting; variations on these mutagenesis strategies will be readily apparent to one of ordinary skill in the art. By these mechanisms, the nucleic acid and protein molecules of the invention may be utilized to generate C. glutamicum or related strains of bacteria expressing mutated MP nucleic acid and protein molecules such that the yield, production, and/or efficiency of production of a desired compound is improved. This desired compound may be any natural product of C. glutamicum, which includes the final products of biosynthesis pathways and intermediates of naturally-occurring metabolic pathways, as well as molecules which do not naturally occur in the metabolism of C. glutamicum, but which are produced by a C. glutamicum strain of the invention. Preferred compounds to be produced by Corynebacterium glutamicum strains are trehalose and/or the amino acids L-lysine and L-methionine.

In one embodiment the metC gene encoding cystathionine â-lyase, the third enzyme in the methionine biosynthetic pathway, was isolated from Corynebacterium glutamicum. The translational product of the gene showed no significant homology with that of metC gene from other organisms. Introduction of the plasmid containing the metC gene into C. glutamicum resulted in 5-fold increase in the activity of cystathionine â-lyase. The protein product now designated MetC encoding a protein product of 35,574 Dalton consisted of 325 amino acids was identical to the previously reported aecD gene except the existence of two different amino acids. Like aecD gene, when present in multiple copies, metC gene conferred resistance to S-(â-aminoethyl)-cysteine which is a toxic lysine analog. However, genetic and biochemical evidences suggest that the natural activity of metC gene product is to mediate methionine biosynthesis in C. glutamicum. Mutant strains of metC were constructed and the strains showed methionine prototrophy. The mutant strains completely lost their ability to show resistance to S-(ã-aminoethyl)-cysteine. These results show that, in addition to the transsulfuration, another biosynthetic pathway—the direct sulfhydrylation pathway is functional in C. glutamicum as a parallel biosynthetic route for methionine.

In yet another embodiment it is also shown that the additional sulfhydrylation pathway is catalyzed by O-acetylhomoserine sulfhydrylase. The presence of the pathway is demonstrated by the isolation of the corresponding metZ (or metY) gene and enzyme. Among the eukaryotes, fungi and yeast species have been reported to have both the transsulfuration and direct sulfhydrylation pathway (Marzluf, 1997). So far, no prokaryotic organism which possesses both pathways has been found. Unlike E. coli which only possesses single biosynthetic route for lysine, C. glutamicum possesses two parallel biosynthetic pathways for the amino acid. The biosynthetic pathway for methionine in C. glutamicum is analogous to that of lysine in that aspect.

The Gene metZ was found because it was located in the upstream region of metA. We sequenced regions upstream and downstream of metA—the gene encoding the enzyme catalysing the first step of methionine biosynthesis (Park, S.-D., Lee, J.-Y., Kim, Y., Kim, J.-H., and Lee, H.-S. (1998) Isolation and analysis of metA, a methionine biosynthetic gene encoding homoserine acetyltransferase in Corynebacterium glutamicum. Mol. Cells 8, 286-294)—to find possible other met genes. It appears that metZ and metA form an operon. Expression of the genes encoding MetA and MetZ leads to overproduction of the corresponding polypeptides as can shown by gel electrophoresis.

Surprisingly, metZ clones can complement methiononine auxotrophic Escherichia coli metB mutant strains. This shows that the protein product of metZ catalyzes a step that can bypass the step catalyzed by the protein product of metB.

MetZ was also disrupted and the mutant strain showed methionine prototrophy. Corynebacterium glutamicum metB and metZ double mutants were also constructed. The double mutant is auxotrophic for methionine. Thus, metZ encodes a protein catalysing the reaction from O-Acetyl-Homoserine to Homocysteine, which is one step in the sulfhydrylation pathway of methionine biosynthesis. Corynebacterium glutamicum contains both, the transsulfuration and the sulfhydrylation pathway of methionine biosynthesis.

Introduction of metZ into C. glutamicum resulted in the expression of a 47,000 Dalton protein. Combined introduction of metZ and metA in C. glutamicum resulted in the appearance of metA and metZ proteins as showed by gel electrophoresis. If the Corynebacterium strain is a lysine overproducer, introduction of a plasmid containing metZ and metA resulted in a lower lysine titer but accumulation of homocysteine and methionine is detected.

In another embodiment metZ and metA were introduced into Corynebacterium glutamicum strains together with the hom gene, encoding the homoserine dehydrogenase, catalysing the conversion from aspartate semialdehyde to homoserine. Different hom genes from different organisms were chosen for this experiment. The Corynebacterium glutamicum hom gene can be used as well as hom genes from other procaryotes like Escherichia coli or Bacillus subtilis or even the hom gene of eukaryotes like Saccharomyces cerevisiae, Shizosaccharomyces pombe, Ashbya gossypii or algae, higher plants or animals. It may be that the hom gene is insensitive against feed back inhibition mediated by any metabolites that occur in the biosynthetic routes of the amino acids of the aspartate familiy, like aspatrate, lysine, threonine or methionine. Such metabolites are for example aspartate, lysine, methionine, threonine, aspartyl-phosphate, aspartate semialdehyd, homoserine, cystathionine, homocysteine or any other metabolite that occurs in this biosynthetic routes. In addition to the metabolites the homoserine dehydrogenase may be insensitive against inhibition by anologes of all those metabolites or even against other compunds involved in this metabolism as there are other amino acids like cysteine or cofactors like vitamin B12 and all of its derivatives and S-adenosylmethionine and its metabolites and derivatives and anologons. The insensitivity of the homoserine dehydrogenase against all these, a part of these or only one of these compounts may either be its natural attitude or it may be the result from one or more mutations that resulted from classical mutation and selection using chemicals or irradiation or other mutagens. The mutations could also be introduced into the hom gene using gene technology, for example the introduction of site specific point mutations or by any method afore mentioned for the MP ore MP encoding DNA-sequences.

When a hom gene was combined with the metZ and metA genes and introduced into a Corynebacterium glutamicum strain that is a lysine overproducer, lysine accumulation was reduced and homocysteine and methionine accumulation was enhanced. A further enhancement of homocysteine and methionine concentrations can be achieved, if a lysine overproducing Corynebacterium glutamicum strain is used and a disruption of the ddh gene or the lysA gene was introduced prior to the transformation with DNA containing a hom gene and metZ and metA in combination. The overproduction of homocysteine and methionine was possible using different sulfur sources. Sulfates, thiosulfates, sulfites and also more reduced sulfur sources like H₂S and sulfides and derivatives could be used. Also organic sulfur sources like methyl mercaptan, thioglycolates, thiocyanates, thiourea, sulfur containing amino acids like cysteine and other sulfur containing compounds can be used to achieve homocysteine and methionine overproduction.

In another embodiment the metC gene was introduced into a Corynebacterium glutamicum strain using methods wich are afore mentioned. The metC gene can be transformed into the strain in combination with other genes like metB, metA and metA. Even the hom gene can be added. If the hom gene, the met C, metA and metB genes were combined on a vector and introduced into a Corynebacterium glutamicum strain homocysteine and methionine overproduction was achieved. The overproduction of homocysteine and methionine was possible using different sulfur sources. Sulfates, thiosulfates, sulfites and also more reduced sulfur sources like H₂S and sulfides and derivatives could be used. Also organic sulfur sources like methyl mercaptan, thioglycolates, thiocyanates, thiourea, sulfur containing amino acids like cysteine and other sulfur containing compounds can be used to achieve homocysteine and methionine overproduction.

This invention is further illustrated by the following examples which should not be construed as limiting. The contents of all references, patent applications, patents, published patent applications, Tables, and the sequence listing cited throughout this application are hereby incorporated by reference.

### EXAMPLE 1

### Preparation of Total Genomic DNA of Corynebacterium glutamicum ATCC 13032

A culture of Corynebacterium glutamicum (ATCC 13032) was grown overnight at 30° C. with vigorous shaking in BHI medium (Difco). The cells were harvested by centrifugation, the supernatant was discarded and the cells were resuspended in 5 ml buffer-I (5% of the original volume of the culture—all indicated volumes have been calculated for 100 ml of culture volume). Composition of buffer-I: 140.34 g/l sucrose, 2.46 g/l MgSO₄×7H₂O, 10 ml/l KH₂PO₄ solution (100 g/l, adjusted to pH 6.7 with KOH), 50 ml/l M12 concentrate (10 g/l (NH₄)₂SO₄, 1 g/l NaCl, 2 g/l MgSO₄×7H₂O, 0.2 g/l CaCl₂, 0.5 g/l yeast extract (Difco), 10 ml/l trace-elements-mix (200 mg/l FeSO₄×H₂O, 10 mg/l ZnSO₄×7H₂O, 3 mg/l MnCl₂×4H₂O, 30 mg/l H₃BO₃ 20 mg/l CoCl₂×6H₂₀, 1 mg/l NiCl₂×6H₂O, 3 mg/l Na₂MoO₄×2H₂O, 500 mg/l complexing agent (EDTA or critic acid), 100 ml/l vitamins-mix (0.2 mg/l biotin, 0.2 mg/l folic acid, 20 mg/l p-amino benzoic acid, 20 mg/l riboflavin, 40 mg/l ca-panthothenate, 140 mg/l nicotinic acid, 40 mg/l pyridoxole hydrochloride, 200 mg/l myo-inositol). Lysozyme was added to the suspension to a final concentration of 2.5 mg/ml. After an approximately 4 h incubation at 37° C., the cell wall was degraded and the resulting protoplasts are harvested by centrifugation. The pellet was washed once with 5 ml buffer-I and once with 5 ml TE-buffer (10 mM Tris-HCl, 1 mM EDTA, pH 8). The pellet was resuspended in 4 ml TE-buffer and 0.5 ml SDS solution (10%) and 0.5 ml NaCl solution (5 M) are added. After adding of proteinase K to a final concentration of 200 μg/ml, the suspension is incubated for ca.18 h at 37° C. The DNA was purified by extraction with phenol, phenol-chloroform-isoamylalcohol and chloroform-isoamylalcohol using standard procedures. Then, the DNA was precipitated by adding 1/50 volume of 3 M sodium acetate and 2 volumes of ethanol, followed by a 30 min incubation at −20° C. and a 30 min centrifugation at 12,000 rpm in a high speed centrifuge using a SS34 rotor (Sorvall). The DNA was dissolved in 1 ml TE-buffer containing 20 μg/ml RNaseA and dialysed at 4° C. against 1000 ml TE-buffer for at least 3 hours. During this time, the buffer was exchanged 3 times. To aliquots of 0.4 ml of the dialysed DNA solution, 0.4 ml of 2 M LiCl and 0.8 ml of ethanol are added. After a 30 min incubation at −20° C., the DNA was collected by centrifugation (13,000 rpm, Biofuge Fresco, Heraeus, Hanau, Germany). The DNA pellet was dissolved in TE-buffer. DNA prepared by this procedure could be used for all purposes, including southern blotting or construction of genomic libraries.

### EXAMPLE 2

### Construction of Genomic Libraries in Escherichia coli of Corynebacterium glutamicum ATCC13032.

Using DNA prepared as described in Example 1, cosmid and plasmid libraries were constructed according to known and well established methods (see e.g., Sambrook, J. et al. (1989) “Molecular Cloning: A Laboratory Manual”, Cold Spring Harbor Laboratory Press, or Ausubel, F. M. et al. (1994) “Current Protocols in Molecular Biology”, John Wiley & Sons.)

Any plasmid or cosmid could be used. Of particular use were the plasmids pBR322 (Sutcliffe, J. G. (1979) Proc. Natl. Acad. Sci. USA, 75:3737-3741); pACYC177 (Change & Cohen (1978) J. Bacteriol 134:1141-1156), plasmids of the pBS series (pBSSK+, pBSSK− and others; Stratagene, LaJolla, USA), or cosmids as SuperCos1 (Stratagene, LaJolla, USA) or Lorist6 (Gibson, T. J., Rosenthal A. and Waterson, R. H. (1987) Gene 53:283-286. Gene libraries specifically for use in C. glutamicum may be constructed using plasmid pSL109 (Lee, H.-S. and A. J. Sinskey (1994) J. Microbiol. Biotechnol. 4: 256-263).

### EXAMPLE 3

### DNA Sequencing and Computational Functional Analysis

Genomic libraries as described in Example 2 were used for DNA sequencing according to standard methods, in particular by the chain termination method using ABI377 sequencing machines (see e.g., Fleischman, R. D. et al. (1995) “Whole-genome Random Sequencing and Assembly of Haemophilus Influenzae Rd., Science, 269:496-512). Sequencing primers with the following nucleotide sequences were used: 5′-GGAAACAGTATGACCATG-3′ or 5′-GTAAAACGACGGCCAGT-3′.

### EXAMPLE 4

### In Vivo Mutagenesis

In vivo mutagenesis of Corynebacterium glutamicum can be performed by passage of plasmid (or other vector) DNA through E. coli or other microorganisms (e.g. Bacillus spp. or yeasts such as Saccharomyces cerevisiae) which are impaired in their capabilities to maintain the integrity of their genetic information. Typical mutator strains have mutations in the genes for the DNA repair system (e.g., mutHLS, mutD, mutT, etc.; for reference, see Rupp, W. D. (1996) DNA repair mechanisms, in: Escherichia coli and Salmonella, p. 2277-2294, ASM: Washington.) Such strains are well known to those of ordinary skill in the art. The use of such strains is illustrated, for example, in Greener, A. and Callahan, M. (1994) Strategies 7: 32-34.

### EXAMPLE 5

### DNA Transfer Between Escherichia coli and Corynebacterium glutamicum

Several Corynebacterium and Brevibacterium species contain endogenous plasmids (as e.g., pHM1519 or pBL1) which replicate autonomously (for review see, e.g., Martin, J. F. et al. (1987) Biotechnology, 5:137-146). Shuttle vectors for Escherichia coli and Corynebacterium glutamicum can be readily constructed by using standard vectors for E. coli (Sambrook, J. et al. (1989), “Molecular Cloning: A Laboratory Manual”, Cold Spring Harbor Laboratory Press or Ausubel, F. M. et al. (1994) “Current Protocols in Molecular Biology”, John Wiley & Sons) to which a origin or replication for and a suitable marker from Corynebacterium glutamicum is added. Such origins of replication are preferably taken from endogenous plasmids isolated from Corynebacterium and Brevibacterium species. Of particular use as transformation markers for these species are genes for kanamycin resistance (such as those derived from the Tn5 or Tn903 transposons) or chloramphenicol (Winnacker, E. L. (1987) “From Genes to Clones—Introduction to Gene Technology, VCH, Weinheim). There are numerous examples in the literature of the construction of a wide variety of shuttle vectors which replicate in both E. coli and C. glutamicum, and which can be used for several purposes, including gene over-expression (for reference, see e.g., Yoshihama, M. et al. (1985) J. Bacteriol. 162:591-597, Martin J. F. et al. (1987) Biotechnology, 5:137-146 and Eikmanns, B. J. et al. (1991) Gene, 102:93-98).

Using standard methods, it is possible to clone a gene of interest into one of the shuttle vectors described above and to introduce such a hybrid vectors into strains of Corynebacterium glutamicum. Transformation of C. glutamicum can be achieved by protoplast transformation (Kastsumata, R. et al. (1984) J. Bacteriol. 159306-311), electroporation (Liebl, E. et al. (1989) FEMS Microbiol. Letters, 53:399-303) and in cases where special vectors are used, also by conjugation (as described e.g. in Schafer, A et al. (1990) J. Bacteriol. 172:1663-1666). It is also possible to transfer the shuttle vectors for C. glutamicum to E. coli by preparing plasmid DNA from C. glutamicum (using standard methods well-known in the art) and transforming it into E. coli. This transformation step can be performed using standard methods, but it is advantageous to use an Mcr-deficient E. coli strain, such as NM522 (Gough & Murray (1983) J. Mol. Biol. 166:1-19).

Genes may be overexpressed in C. glutamicum strains using plasmids which comprise pCG1 (U.S. Pat. No. 4,617,267) or fragments thereof, and optionally the gene for kanamycin resistance from TN903 (Grindley, N. D. and Joyce, C. M. (1980) Proc. Natl. Acad. Sci. USA 77(12): 7176-7180). In addition, genes may be overexpressed in C. glutamicum strains using plasmid pSL109 (Lee, H.-S. and A. J. Sinskey (1994) J. Microbiol. Biotechnol. 4: 256-263).

Aside from the use of replicative plasmids, gene overexpression can also be achieved by integration into the genome. Genomic integration in C. glutamicum or other Corynebacterium or Brevibacterium species may be accomplished by well-known methods, such as homologous recombination with genomic region(s), restriction endonuclease mediated integration (REMI) (see, e.g., DE Patent 19823834), or through the use of transposons. It is also possible to modulate the activity of a gene of interest by modifying the regulatory regions (e.g., a promoter, a repressor, and/or an enhancer) by sequence modification, insertion, or deletion using site-directed methods (such as homologous recombination) or methods based on random events (such as transposon mutagenesis or REMI). Nucleic acid sequences which function as transcriptional terminators may also be inserted 3′ to the coding region of one or more genes of the invention; such terminators are well-known in the art and are described, for example, in Winnacker, E. L. (1987) From Genes to Clones—Introduction to Gene Technology. VCH: Weinheim.

### EXAMPLE 6

### Assessment of the Expression of the Mutant Protein

Observations of the activity of a mutated protein in a transformed host cell rely on the fact that the mutant protein is expressed in a similar fashion and in a similar quantity to that of the wild-type protein. A useful method to ascertain the level of transcription of the mutant gene (an indicator of the amount of mRNA available for translation to the gene product) is to perform a Northern blot (for reference see, for example, Ausubel et al. (1988) Current Protocols in Molecular Biology, Wiley: New York), in which a primer designed to bind to the gene of interest is labeled with a detectable tag (usually radioactive or chemiluminescent), such that when the total RNA of a culture of the organism is extracted, run on gel, transferred to a stable matrix and incubated with this probe, the binding and quantity of binding of the probe indicates the presence and also the quantity of mRNA for this gene. This information is evidence of the degree of transcription of the mutant gene. Total cellular RNA can be prepared from Corynebacterium glutamicum by several methods, all well-known in the art, such as that described in Bormann, E. R. et al. (1992) Mol. Microbiol. 6: 317-326.

To assess the presence or relative quantity of protein translated from this mRNA, standard techniques, such as a SDS-Polyacrylamide Gelelectrophoresis and Western blot, may be employed (see, for example, Ausubel et al. (1988) Current Protocols in Molecular Biology, Wiley: New York). In this process, total cellular proteins are extracted, separated by gel electrophoresis, transferred to a matrix such as nitrocellulose, and incubated with a probe, such as an antibody, which specifically binds to the desired protein. This probe is generally tagged with a chemiluminescent or calorimetric label which may be readily detected. The presence and quantity of label observed indicates the presence and quantity of the desired mutant protein present in the cell.

### EXAMPLE 7

### Growth of Escherichia coli and Genetically Modified Corynebacterium glutamicum—Media and Culture Conditions

E. coli strains are routinely grown in MB and LB broth, respectively (Follettie, M. T., Peoples, O., Agoropoulou, C., and Sinskey, A J. (1993) Gene structure and expression of the Corynebacterium flavum N13 ask-asd operon. J. Bacteriol. 175, 4096-4103). Minimal media for E. coli is M9 and modified MCGC (Yoshihama, M., Higashiro, K., Rao, E. A., Akedo, M., Shanabruch, W G., Follettie, M. T., Walker, G. C., and Sinskey, A. J. (1985) Cloning vector system for Corynebacterium glutamicum. J. Bacteriol. 162, 591-507), respectively. Glucose was added a final concentration of 1%. Antibiotics were added in the following amounts (micrograms per milliliter): ampicillin, 50; kanamycin, 25; nalidixic acid, 25. Amino acids, vitamins, and other supplements were added in the following amounts: methionine, 9.3 mM; arginine, 9.3 mM; histidine, 9.3 mM; thiamine, 0.05 mM. E. coli cells were routinely grown at 37° C., respectively.

Genetically modified Corynebacteria are cultured in synthetic or natural growth media. A number of different growth media for Corynebacteria are both well-known and readily available (Lieb et al. (1989) Appl. Microbiol. Biotechnol., 32:205-210; von der Osten et al. (1998) Biotechnology Letters, 11:11-16; Pat. No. DE 4,120,867; Liebl (1992) “The Genus Corynebacterium, in: The Procaryotes, Volume II, Balows, A. et al., eds. Springer-Verlag). These media consist of one or more carbon sources, nitrogen sources, inorganic salts, vitamins and trace elements. Preferred carbon sources are sugars, such as mono-, di-, or polysaccharides. For example, glucose, fructose, mannose, galactose, ribose, sorbose, ribulose, lactose, maltose, sucrose, raffinose, starch or cellulose serve as very good carbon sources. It is also possible to supply sugar to the media via complex compounds such as molasses or other by-products from sugar refinement. It can also be advantageous to supply mixtures of different carbon sources. Other possible carbon sources are alcohols and organic acids, such as methanol, ethanol, acetic acid or lactic acid. Nitrogen sources are usually organic or inorganic nitrogen compounds, or materials which contain these compounds. Exemplary nitrogen sources include ammonia gas or ammonia salts, such as NH₄Cl or (NH₄)₂SO₄, NH₄OH, nitrates, urea, amino acids or complex nitrogen sources like corn steep liquor, soy bean flour, soy bean protein, yeast extract, meat extract and others.

The overproduction of sulfur containig amino acids like homocysteine and methionine was possible using different sulfur sources. Sulfates, thiosulfates, sulfites and also more reduced sulfur sources like H₂S and sulfides and derivatives can be used. Also organic sulfur sources like methyl mercaptan, thioglycolates, thiocyanates, thiourea, sulfur containing amino acids like cysteine and other sulfur containing compounds can be used to achieve homocysteine and methionine overproduction. Inorganic salt compounds which may be included in the media include the chloride-, phosphorous- or sulfate-salts of calcium, magnesium, sodium, cobalt, molybdenum, potassium, manganese, zinc, copper and iron. Chelating compounds can be added to the medium to keep the metal ions in solution. Particularly useful chelating compounds include dihydroxyphenols, like catechol or protocatechuate, or organic acids, such as citric acid. It is typical for the media to also contain other growth factors, such as vitamins or growth promoters, examples of which include biotin, riboflavin, thiamin, folic acid, nicotinic acid, pantothenate and pyridoxin. Growth factors and salts frequently originate from complex media components such as yeast extract, molasses, corn steep liquor and others. The exact composition of the media compounds depends strongly on the immediate experiment and is individually decided for each specific case. Information about media optimization is available in the textbook “Applied Microbiol. Physiology, A Practical Approach (eds. P. M. Rhodes, P. F. Stanbury, IRL Press (1997) pp. 53-73, ISBN 0 19 963577 3). It is also possible to select growth media from commercial suppliers, like standard 1 (Merck) or BHI (grain heart infusion, DIFCO) or others.

All medium components are sterilized, either by heat (20 minutes at 1.5 bar and 121° C.) or by sterile filtration. The components can either be sterilized together or, if necessary, separately. All media components can be present at the beginning of growth, or they can optionally be added continuously or batchwise.

Culture conditions are defined separately for each experiment. The temperature should be in a range between 15° C. and 45° C. The temperature can be kept constant or can be altered during the experiment. The pH of the medium should be in the range of 5 to 8.5, preferably around 7.0, and can be maintained by the addition of buffers to the media. An exemplary buffer for this purpose is a potassium phosphate buffer. Synthetic buffers such as MOPS, HEPES, ACES and others can alternatively or simultaneously be used. It is also possible to maintain a constant culture pH through the addition of NaOH or NH₄OH during growth. If complex medium components such as yeast extract are utilized, the necessity for additional buffers may be reduced, due to the fact that many complex compounds have high buffer capacities. If a fermentor is utilized for culturing the micro-organisms, the pH can also be controlled using gaseous ammonia.

The incubation time is usually in a range from several hours to several days. This time is selected in order to permit the maximal amount of product to accumulate in the broth. The disclosed growth experiments can be carried out in a variety of vessels, such as microtiter plates, glass tubes, glass flasks or glass or metal fermentors of different sizes. For screening a large number of clones, the microorganisms should be cultured in microtiter plates, glass tubes or shake flasks, either with or without baffles. Preferably 100 ml shake flasks are used, filled with 10% (by volume) of the required growth medium. The flasks should be shaken on a rotary shaker (amplitude 25 mm) using a speed-range of 100-300 rpm. Evaporation losses can be diminished by the maintenance of a humid atmosphere; alternatively, a mathematical correction for evaporation losses should be performed.

If genetically modified clones are tested, an unmodified control clone or a control clone containing the basic plasmid without any insert should also be tested. The medium is inoculated to an OD₆₀₀ of 0.5-1.5 using cells grown on agar plates, such as CM plates (10 g/l glucose, 2,5 g/l NaCl, 2 g/l urea, 10 g/l polypeptone, 5 g/l yeast extract, 5 g/l meat extract, 22 g/l NaCl, 2 g/l urea, 10 g/l polypeptone, 5 g/l yeast extract, 5 g/l meat extract, 22 g/l agar, pH 6.8 with 2M NaOH) that had been incubated at 30° C. Inoculation of the media is accomplished by either introduction of a saline suspension of C. glutamicum cells from CM plates or addition of a liquid preculture of this bacterium.

### EXAMPLE 8

### In vitro Analysis of the Function of Mutant Proteins

The determination of activities and kinetic parameters of enzymes is well established in the art. Experiments to determine the activity of any given altered enzyme must be tailored to the specific activity of the wild-type enzyme, which is well within the ability of one of ordinary skill in the art. Overviews about enzymes in general, as well as specific details concerning structure, kinetics, principles, methods, applications and examples for the determination of many enzyme activities may be found, for example, in the following references: Dixon, M., and Webb, E. C., (1979) Enzymes. Longmans: London; Fersht, (1985) Enzyme Structure and Mechanism. Freeman: New York; Walsh, (1979) Enzymatic Reaction Mechanisms. Freeman: San Francisco; Price, N. C., Stevens, L. (1982) Fundamentals of Enzymology. Oxford Univ. Press: Oxford; Boyer, P. D., ed. (1983) The Enzymes, 3^{rd} ed. Academic Press: New York; Bisswanger, H., (1994) Enzymkinetik, 2nd ed. VCH: Weinheim (ISBN 3527300325); Bergmeyer, H. U., Bergmeyer, J., Grail, M., eds. (1983-1986) Methods of Enzymatic Analysis, 3^{rd} ed., vol. I-XII, Verlag Chemie: Weinheim; and Ullmann's Encyclopedia of Industrial Chemistry (1987) vol. A9, “Enzymes”. VCH: Weinheim, p. 352-363.

The activity of proteins which bind to DNA can be measured by several well-established methods, such as DNA band-shift assays (also called gel retardation assays). The effect of such proteins on the expression of other molecules can be measured using reporter gene assays (such as that described in Kolmar, H. et al. (1995) EMBO J. 14: 3895-3904 and references cited therein). Reporter gene test systems are well known and established for applications in both pro- and eukaryotic cells, using enzymes such as beta-galactosidase, green fluorescent protein, and several others.

The determination of activity of membrane-transport proteins can be performed according to techniques such as those described in Gennis, R. B. (1989) “Pores, Channels and Transporters”, in Biomembranes, Molecular Structure and Function, Springer: Heidelberg, p. 85-137; 199-234; and 270-322.

### EXAMPLE 9

### Analysis of Impact of Mutant Protein on the Production of the Desired Product

The effect of the genetic modification in C. glutamicum on production of a desired compound (such as an amino acid) can be assessed by growing the modified microorganism under suitable conditions (such as those described above) and analyzing the medium and/or the cellular component for increased production of the desired product (i.e., an amino acid). Such analysis techniques are well known to one of ordinary skill in the art, and include spectroscopy, thin layer chromatography, staining methods of various kinds, enzymatic and microbiological methods, and analytical chromatography such as high performance liquid chromatography (see, for example, Ullman, Encyclopedia of Industrial Chemistry, vol. A2, p. 89-90 and p. 443-613, VCH: Weinheim (1985); Fallon, A. et al., (1987) “Applications of HPLC in Biochemistry” in: Laboratory Techniques in Biochemistry and Molecular Biology, vol. 17; Rehm et al. (1993) Biotechnology, vol. 3, Chapter III: “Product recovery and purification”, page 469-714, VCH: Weinheim; Belter, P. A. et al. (1988) Bioseparations: downstream processing for biotechnology, John Wiley and Sons; Kennedy, J. F. and Cabral, J. M. S. (1992) Recovery processes for biological materials, John Wiley and Sons; Shaeiwitz, J. A. and Henry, J. D. (1988) Biochemical separations, in: Ulmann's Encyclopedia of Industrial Chemistry, vol. B3, Chapter 11, page 1-27, VCH: Weinheim; and Dechow, F. J. (1989) Separation and purification techniques in biotechnology, Noyes Publications.)

In addition to the measurement of the final product of fermentation, it is also possible to analyze other components of the metabolic pathways utilized for the production of the desired compound, such as intermediates and side-products, to determine the overall efficiency of production of the compound. Analysis methods include measurements of nutrient levels in the medium (e.g., sugars, hydrocarbons, nitrogen sources, phosphate, and other ions), measurements of biomass composition and growth, analysis of the production of common metabolites of biosynthetic pathways, and measurement of gasses produced during fermentation. Standard methods for these measurements are outlined in Applied Microbial Physiology, A Practical Approach, P. M. Rhodes and P. F. Stanbury, eds., IRL Press, p. 103-129; 131-163; and 165-192 (ISBN: 0199635773) and references cited therein.

### EXAMPLE 10

### Purification of the Desired Product from C. glutamicum Culture

Recovery of the desired product from the C. glutamicum cells or supernatant of the above-described culture can be performed by various methods well known in the art. If the desired product is not secreted from the cells, the cells can be harvested from the culture by low-speed centrifugation, the cells can be lysed by standard techniques, such as mechanical force or sonication. The cellular debris is removed by centrifugation, and the supernatant fraction containing the soluble proteins is retained for further purification of the desired compound. If the product is secreted from the C. glutamicum cells, then the cells are removed from the culture by low-speed centrifugation, and the supernate fraction is retained for further purification.

The supernatant fraction from either purification method is subjected to chromatography with a suitable resin, in which the desired molecule is either retained on a chromatography resin while many of the impurities in the sample are not, or where the impurities are retained by the resin while the sample is not. Such chromatography steps may be repeated as necessary, using the same or different chromatography resins. One of ordinary skill in the art would be well-versed in the selection of appropriate chromatography resins and in their most efficacious application for a particular molecule to be purified. The purified product may be concentrated by filtration or ultrafiltration, and stored at a temperature at which the stability of the product is maximized.

There are a wide array of purification methods known to the art and the preceding method of purification is not meant to be limiting. Such purification techniques are described, for example, in Bailey, J. E. & Ollis, D. F. Biochemical Engineering Fundamentals, McGraw-Hill: New York (1986).

The identity and purity of the isolated compounds may be assessed by techniques standard in the art. These include high-performance liquid chromatography (HPLC), spectroscopic methods, staining methods, thin layer chromatography, NIRS, enzymatic assay, or microbiologically. Such analysis methods are reviewed in: Patek et al. (1994) Appl. Environ. Microbiol. 60: 133-140; Malakhova et al. (1996) Biotekhnologiya 11: 27-32; and Schmidt et al. (1998) Bioprocess Engineer. 19: 67-70. Ulmann's Encyclopedia of Industrial Chemistry, (1996) vol. A27, VCH: Weinheim, p. 89-90, p. 521-540, p. 540-547, p. 559-566, 575-581 and p. 581-587; Michal, G. (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, vol. 17.

### EXAMPLE 11

### Analysis of the Gene Sequences of the Invention

The comparison of sequences and determination of percent homology between two sequences are art-known techniques, and can be accomplished using a mathematical algorithm, such as the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to MP nucleic acid molecules of the invention. BLAST protein searches can be performed with the XBLAST program, score=50, wordlength=3 to obtain amino acid sequences homologous to MP protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, one of ordinary skill in the art will know how to optimize the parameters of the program (e.g., XBLAST and NBLAST) for the specific sequence being analyzed.

Another example of a mathematical algorithm utilized for the comparison of sequences is the algorithm of Meyers and Miller ((1988) Comput. Appl. Biosci. 4: 11-17). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art, and include ADVANCE and ADAM. described in Torelli and Robotti (1994) Comput. Appl. Biosci. 10:3-5; and FASTA, described in Pearson and Lipman (1988) P.N.A.S. 85:2444-8.

The percent homology between two amino acid sequences can also be accomplished using the GAP program in the GCG software package (available at http://www.gcg.com), using either a Blosum 62 matrix or a PAM250 matrix, and a gap weight of 12, 10, 8, 6, or 4 and a length weight of 2, 3, or 4. The percent homology between two nucleic acid sequences can be accomplished using the GAP program in the GCG software package, using standard parameters, such as a gap weight of 50 and a length weight of 3.

A comparative analysis of the gene sequences of the invention with those present in Genbank has been performed using techniques known in the art (see, e.g., Bexevanis and Ouellette, eds. (1998) Bioinformatics: A Practical Guide to the Analysis of Genes and Proteins. John Wiley and Sons: New York).

The gene sequences of the invention were compared on basis of their amino acid sequences to known genes by using the program CLUSTAL (Higgins et al. (1996) Using CLUSTAL for multiple sequence alignments, Methods in Enzymology 266, 383-402) using the standard parameters (PAIRWISE ALIGNMENT PARAMETERS: Gap penalty=3, K-tuple (word) size=1, No. of top diagonals=5, Window size=5; MULTIPLE ALIGNMENT PARAMETERS: Gap Opening Penalty=10.00, Gap Extension Penalty=0.05, Protein weight matrix=PAM250). Homology between two sequences is the function of the number of identical positions in all sequences (i.e. % homology=number of identical positions/total number of positions×100). The results of this analysis are set forth in Table 3.

### EXAMPLE 12

### Construction and Operation of DNA Microarrays

The sequences of the invention may additionally be used in the construction and application of DNA microarrays (the design, methodology, and uses of DNA arrays are well known in the art, and are described, for example, in Schena, M. et al. (1995) Science 270: 467-470; Wodicka, L. et al. (1997) Nature Biotechnology 15: 1359-1367; DeSaizieu, A. et al. (1998) Nature Biotechnology 16: 45-48; and DeRisi, J. L. et al. (1997) Science 278: 680-686).

DNA microarrays are solid or flexible supports consisting of nitrocellulose, nylon, glass, silicone, or other materials. Nucleic acid molecules may be attached to the surface in an ordered manner. After appropriate labeling, other nucleic acids or nucleic acid mixtures can be hybridized to the immobilized nucleic acid molecules, and the label may be used to monitor and measure the individual signal intensities of the hybridized molecules at defined regions. This methodology allows the simultaneous quantification of the relative or absolute amount of all or selected nucleic acids in the applied nucleic acid sample or mixture. DNA microarrays, therefore, permit an analysis of the expression of multiple (as many as 6800 or more) nucleic acids in parallel (see, e.g., Schena, M. (1996) BioEssays 18(5): 427-431).

The sequences of the invention may be used to design oligonucleotide primers which are able to amplify defined regions of one or more C. glutamicum genes by a nucleic acid amplification reaction such as the polymerase chain reaction. The choice and design of the 5′ or 3′ oligonucleotide primers or of appropriate linkers allows the covalent attachment of the resulting PCR products to the surface of a support medium described above (and also described, for example, Schena, M. et al. (1995) Science 270: 467-470).

Nucleic acid microarrays may also be constructed by in situ oligonucleotide synthesis as described by Wodicka, L. et al. (1997) Nature Biotechnology 15: 1359-1367. By photolithographic methods, precisely defined regions of the matrix are exposed to light. Protective groups which are photolabile are thereby activated and undergo nucleotide addition, whereas regions that are masked from light do not undergo any modification. Subsequent cycles of protection and light activation permit the synthesis of different oligonucleotides at defined positions. Small, defined regions of the genes of the invention may be synthesized on microarrays by solid phase oligonucleotide synthesis.

The nucleic acid molecules of the invention present in a sample or mixture of nucleotides may be hybridized to the microarrays. These nucleic acid molecules can be labeled according to standard methods. In brief, nucleic acid molecules (e.g., mRNA molecules or DNA molecules) are labeled by the incorporation of isotopically or fluorescently labeled nucleotides, e.g., during reverse transcription or DNA synthesis. Hybridization of labeled nucleic acids to microarrays is described (e.g., in Schena, M. et al. (1995) supra; Wodicka, L. et al. (1997), supra; and DeSaizieu A. et al. (1998), supra). The detection and quantification of the hybridized molecule are tailored to the specific incorporated label. Radioactive labels can be detected, for example, as described in Schena, M. et al. (1995) supra) and fluorescent labels may be detected, for example, by the method of Shalon et al. (1996) Genome Research 6: 639-645).

The application of the sequences of the invention to DNA microarray technology, as described above, permits comparative analyses of different strains of C. glutamicum or other Corynebacteria. For example, studies of inter-strain variations based on individual transcript profiles and the identification of genes that are important for specific and/or desired strain properties such as pathogenicity, productivity and stress tolerance are facilitated by nucleic acid array methodologies. Also, comparisons of the profile of expression of genes of the invention during the course of a fermentation reaction are possible using nucleic acid array technology.

### EXAMPLE 13

### Analysis of the Dynamics of Cellular Protein Populations (Proteomics)

The genes, compositions, and methods of the invention may be applied to study the interactions and dynamics of populations of proteins, termed ‘proteomics’. Protein populations of interest include, but are not limited to, the total protein population of C. glutamicum (e.g., in comparison with the protein populations of other organisms), those proteins which are active under specific environmental or metabolic conditions (e.g., during fermentation, at high or low temperature, or at high or low pH), or those proteins which are active during specific phases of growth and development.

Protein populations can be analyzed by various well-known techniques, such as gel electrophoresis. Cellular proteins may be obtained, for example, by lysis or extraction, and may be separated from one another using a variety of electrophoretic techniques. Sodium dodecyl sulfate polyacrylamide gel electrophoresis (SDS-PAGE) separates proteins largely on the basis of their molecular weight. Isoelectric focusing polyacrylamide gel electrophoresis (IEF-PAGE) separates proteins by their isoelectric point (which reflects not only the amino acid sequence but also posttranslational modifications of the protein). Another, more preferred method of protein analysis is the consecutive combination of both IEF-PAGE and SDS-PAGE, known as 2-D-gel electrophoresis (described, for example, in Hermann et al. (1998) Electrophoresis 19: 3217-3221; Fountoulakis et al. (1998) Electrophoresis 19: 1193-1202; Langen et al. (1997) Electrophoresis 18: 1184-1192; Antelmann et al. (1997) Electrophoresis 18: 1451-1463). Other separation techniques may also be utilized for protein separation, such as capillary gel electrophoresis; such techniques are well known in the art.

Proteins separated by these methodologies can be visualized by standard techniques, such as by staining or labeling. Suitable stains are known in the art, and include Coomassie Brilliant Blue, silver stain, or fluorescent dyes such as Sypro Ruby (Molecular Probes). The inclusion of radioactively labeled amino acids or other protein precursors (e.g., ³⁵S-methionine, ³⁵S-cysteine, ¹⁴C-labelled amino acids, ¹⁵N-amino acids, ¹⁵NO₃ or 15NH₄⁺ or ¹³C-labelled amino acids) in the medium of C. glutamicum permits the labeling of proteins from these cells prior to their separation. Similarly, fluorescent labels may be employed. These labeled proteins can be extracted, isolated and separated according to the previously described techniques.

Proteins visualized by these techniques can be further analyzed by measuring the amount of dye or label used. The amount of a given protein can be determined quantitatively using, for example, optical methods and can be compared to the amount of other proteins in the same gel or in other gels. Comparisons of proteins on gels can be made, for example, by optical comparison, by spectroscopy, by image scanning and analysis of gels, or through the use of photographic films and screens. Such techniques are well-known in the art.

To determine the identity of any given protein, direct sequencing or other standard techniques may be employed. For example, N- and/or C-terminal amino acid sequencing (such as Edman degradation) may be used, as may mass spectrometry (in particular MALDI or ESI techniques (see, e.g., Langen et al. (1997) Electrophoresis 18: 1184-1192)). The protein sequences provided herein can be used for the identification of C. glutamicum proteins by these techniques.

The information obtained by these methods can be used to compare patterns of protein presence, activity, or modification between different samples from various biological conditions (e.g., different organisms, time points of fermentation, media conditions, or different biotopes, among others). Data obtained from such experiments alone, or in combination with other techniques, can be used for various applications, such as to compare the behavior of various organisms in a given (e.g., metabolic) situation, to increase the productivity of strains which produce fine chemicals or to increase the efficiency of the production of fine chemicals.

Equivalents

Those of ordinary skill in the art will recognize, or will be able to ascertain using no more than routine experimentation, many equivalents to the specific embodiments of the invention described herein. Such equivalents are intended to be encompassed by the following claims.

1. An isolated Corynebacterium glutamicum nucleic acid molecule selected from the group consisting of those sequences set forth as odd-numbered SEQ ID NOs of the Sequence Listing, or a portion thereof, as set forth in Table 1.

2. An isolated nucleic acid molecule which encodes a polypeptide sequence selected from the group consisting of those sequences set forth as even-numbered SEQ ID NOs of the Sequence Listing, as set forth in Table 1.

3. An isolated nucleic acid molecule which encodes a naturally occurring allelic variant of a polypeptide selected from the group of amino acid sequences consisting of those sequences set forth as even-numbered SEQ ID NOs of the Sequence Listing, as set forth in Table 1.

4. An isolated nucleic acid molecule comprising a nucleotide sequence which is at least 63% homologous on basis of its amino acid sequence to a nucleotide sequence selected from the group consisting of those sequences which encode for an amino acid sequence as set forth as SEQ ID NO 2 of the Sequence Listing, or a portion thereof, or sequence which is at least 71% homologous on basis of its amino acid sequence to a nucleotide sequence selected from the group consisting of those sequences which encode for an amino acid sequence as set forth as SEQ ID NO 4 of the Sequence Listing, or a portion thereof.

5. An isolated nucleic acid molecule comprising a fragment of at least 15 nucleotides of a nucleic acid comprising a nucleotide sequence selected from the group consisting of those sequences set forth as odd-numbered SEQ ID NOs of the Sequence Listing, as set forth in Table 1.

6. An isolated nucleic acid molecule which hybridizes to the nucleic acid molecule of any one of claims 1-5 under stringent conditions.

7. An isolated nucleic acid molecule comprising the nucleic acid molecule of any one of claims 1-6 or a portion thereof and a nucleotide sequence encoding a heterologous polypeptide.

8. A DNA-construct comprising the nucleic acid molecule of any one of claims 1-7 and a regulatory sequence.

9. A vector comprising the nucleic acid molecule of any one of claims 1-7.

10. A vector of claim 9 comprising in addition one ore more copies of the same or different nucleic acid molecule of table 4 provided the nucleic acid molecule pertains methionine or of table 5 provided the nucleic acid molecule pertains trehalose.

11. The vector of any one of the claims 9 or 10, which is an expression vector.

12. A host cell transfected with the expression vector of claim 11.

13. The host cell of claim 12, wherein said cell is a microorganism.

14. The host cell of claim 13, wherein said cell belongs to the genus Corynebacterium or Brevibacterium.

15. The host cell of claim 12, wherein the expression of said nucleic acid molecule results in the modulation in production of a fine chemical from said cell.

16. The host cell of claim 15, wherein said fine chemical is selected from the group consisting of: organic acids, non-proteinogenic amino acids, purine and pyrimidine bases, nucleosides, nucleotides, lipids, saturated and unsaturated fatty acids, diols, carbohydrates, aromatic compounds, vitamins, cofactors, polyketides, and enzymes.

17. A method of producing a polypeptide comprising culturing the host cell of claim 12 in an appropriate culture medium to, thereby, produce the polypeptide.

18. An isolated polypeptide comprising an amino acid sequence selected from the group consisting of those sequences set forth as even-numbered SEQ ID NOs of the Sequence Listing, as set forth in Table 1.

19. An isolated polypeptide comprising a naturally occurring allelic variant of a polypeptide comprising an amino acid sequence selected from the group consisting of those sequences set forth as even-numbered SEQ ID NOs of the Sequence Listing, or a portion thereof, as set forth in Table 1.

20. The isolated polypeptide of any of claims 18 or 19, further comprising heterologous amino acid sequences.

21. An isolated polypeptide which is encoded by a nucleic acid molecule comprising a nucleotide sequence which is at least 63% homologous to a nucleic acid selected from the group consisting of those sequences set forth as odd-numbered SEQ ID NOs of the Sequence Listing, as set forth in Table 1.

22. An isolated polypeptide comprising an amino acid sequence which is at least 63% homologous to an amino acid sequence selected from the group consisting of those sequences set forth as even-numbered SEQ ID NOs of the Sequence Listing, as set forth in Table 1.

23. A method for producing a fine chemical, comprising culturing a cell containing a vector of claim 11 such that the fine chemical is produced.

24. The method of claim 23, wherein said method further comprises the step of recovering the fine chemical from said culture.

25. The method of claim 23, wherein said method further comprises the step of transfecting said cell with the vector of claim 11 to result in a cell containing said vector.

26. The method of claim 23, wherein said cell belongs to the genus Corynebacterium or Brevibacterium.

27. The method of claim 23, wherein said cell is selected from the group consisting of: Corynebacterium glutamicum, Corynebacterium herculis, Corynebacterium, lilium, Corynebacterium acetoacidophilum, Corynebacterium acetoglutamicum, Corynebacterium acetophilum, Corynebacterium ammoniagenes, Corynebacterium fujiokense, Corynebacterium nitrilophilus, Brevibacterium ammoniagenes, Brevibacterium butanicum, Brevibacterium divaricatum, Brevibacterium flavum, Brevibacterium healii, Brevibacterium ketoglutamicum, Brevibacterium ketosoreductum, Brevibacterium lactofermentum, Brevibacterium linens, Brevibacterium paraffinolyticum, and those strains set forth in Table 2.

28. The method of claim 23, wherein expression of the nucleic acid molecule from said vector results in modulation of production of said fine chemical.

29. The method of claim 23, wherein said fine chemical is selected from the group consisting of: organic acids, non-proteinogenic amino acids, purine and pyrimidine bases, nucleosides, nucleotides, lipids, saturated and unsaturated fatty acids, diols, carbohydrates, aromatic compounds, vitamins, cofactors, polyketides, and enzymes.

30. The method of claim 23, wherein said fine chemical is an amino acid or a carbohydrate.

31. The method of claim 30, wherein said amino acid carbohydrate is drawn from the group consisting of: methionine or trehalose.

32. A method for producing a fine chemical, comprising culturing a cell whose genomic DNA has been altered by the inclusion of a nucleic acid molecule of any one of claims 1-7.

33. A method for producing a fine chemical of claim 32 comprising in addition one ore more copies of the same or different nucleic acid molecule of table 4 provided the nucleic acid molecule pertains methionine or of table 5 provided the nucleic acid molecule pertains trehalose.

34. A method for diagnosing the presence or activity of Corynebacterium diphtheriae in a subject, comprising detecting the presence of one or more of SEQ ID NOs 1 through 4 of the Sequence Listing in the subject, thereby diagnosing the presence or activity of Corynebacterium diphtheriae in the subject.

35. A host cell comprising a nucleic acid molecule selected-from the group consisting of the nucleic acid molecules set forth as odd-numbered SEQ ID NOs of the Sequence Listing, wherein the nucleic acid molecule is disrupted.

36. A host cell comprising a nucleic acid molecule selected from the group consisting of the nucleic acid molecules set forth as odd-numbered SEQ ID NOs in the Sequence Listing, wherein the nucleic acid molecule comprises one or more nucleic acid modifications from the sequence set forth as odd-numbered SEQ ID NOs of the Sequence Listing.

37. A host cell comprising a nucleic acid molecule selected from the group consisting of the nucleic acid molecules set forth as odd-numbered SEQ ID NOs of the Sequence Listing, wherein the regulatory region of the nucleic acid molecule is modified relative to the wild-type regulatory region of the molecule.


SEQUENCE LISTING (46 sequences)

SEQ ID NO: 1 - DNA, 3793 bp, Corynebacterium glutamicum
  CDS (101)..(3763)
  Xaa(834)= Met or Ile
agactagtggcgctttgcctgtgttgcttaggcggcgttgaaaatgaactacgaatgaaaagttcgggaattgtctaatccgtactaagctgtctacacaatgtctacttcagttacttcaccagcccacaacaacgcacattcctccgaatttttggatgcgttggcaaaccatgtgttgatcggcgacggcgccatgggcacccagctccaaggctttgacctggacgtggaaaaggatttccttgatctggaggggtgtaatgagattctcaacgacacccgccctgatgtgttgaggcagattcaccgcgcctactttgaggcgggagctgacttggttgagaccaatacttttggttgcaacctgccgaacttggcggattatgacatcgctgatcgttgccgtgagcttgcctacaagggcactgcagtggctagggaagtggctgatgagatggggccgggccgaaacggcatgcggcgtttcgtggttggttccctgggacctggaacgaagcttccatcgctgggccatgcaccgtatgcagatttgcgtgggcactacaaggaagcagcgcttggcatcatcgacggtggtggcgatgcctttttgattgagactgctcaggacttgcttcaggtcaaggctgcggttcacggcgttcaagatgccatggctgaacttgatacattcttgcccattatttgccacgtcaccgtagagaccaccggcaccatgctcatgggttctgagatcggtgccgcgttgacagcgctgcagccactgggtatcgacatgattggtctgaactgcgccaccggcccagatgagatgagcgagcacctgcgttacctgtccaagcacgccgatattcctgtgtcggtgatgcctaacgcaggtcttcctgtcctgggtaaaaacggtgcagaatacccacttgaggctgaggatttggcgcaggcgctggctggattcgtctccgaatatggcctgtccatggtgggtggttgttgtggcaccacacctgagcacatccgtgcggtccgcgatgcggtggttggtgttccagagcaggaaacctccacactgaccaagatccctgcaggccctgttgagcaggcctcccgcgaggtggagaaagaggactccgtcgcgtcgctgtacacctcggtgccattgtcccaggaaaccggcatttccatgatcggtgagcgcaccaactccaacggttccaaggcattccgtgaggcaatgctgtctggcgattgggaaaagtgtgtggatattgccaagcagcaaacccgcgatggtgcacacatgctggatctttgtgtggattacgtgggacgagacggcaccgccgatatggcgaccttggcagcacttcttgctaccagctccactttgccaatcatgattgactccaccgagccagaggttattcgcacaggccttgagcacttgggtggacgaagcatcgttaactccgtcaactttgaagacggcgatggccctgagtcccgctaccagcgcatcatgaaactggtaaagcagcacggtgcggccgtggttgcgctgaccattgatgaggaaggccaggcacgtaccgctgagcacaaggtgcgcattgctaaacgactgattgacgatatcaccggcagctacggcctggatatcaaagacatcgttgtggactgcctgaccttcccgatctctactggccaggaagaaaccaggcgagatggcattgaaaccatcgaagccatccgcgagctgaagaagctctacccagaaatccacaccaccctgggtctgtccaatatttccttcggcctgaaccctgctgcacgccaggttcttaactctgtgttcctcaatgagtgcattgaggctggtctggactctgcgattgcgcacagctccaagattttgccgatgaaccgcattgatgatcgccagcgcgaagtggcgttggatatggtctatgatcgccgcaccgaggattacgatccgctgcaggaattcatgcagctgtttgagggcgtttctgctgccgatgccaaggatgctcgcgctgaacagctggccgctatgcctttgtttgagcgtttggcacagcgcatcatcgacggcgataagaatggccttgaggatgatctggaagcaggcatgaaggagaagtctcctattgcgatcatcaacgaggaccttctcaacggcatgaagaccgtgggtgagctgtttggttccggacagatgcagctgccattcgtgctgcaatcggcagaaaccatgaaaactgcggtggcctatttggaaccgttcatggaagaggaagcagaagctaccggatctgcgcaggcagagggcaagggcaaaatcgtcgtggccaccgtcaagggtgacgtgcacgatatcggcaagaacttggtggacatcattttgtccaacaacggttacgacgtggtgaacttgggcatcaagcagccactgtccgccatgttggaagcagcggaagaacacaaagcagacgtcatcggcatgtcgggacttcttgtgaagtccaccgtggtgatgaaggaaaaccttgaggagatkaacaacgccggcgcatccaattacccagtcattttgggtggcgctgcgctgacgcgtacctacgtggaaaacgatctcaacgaggtgtacaccggtgaggtgtactacgcccgtgatgctttcgagggcctgcgcctgatggatgaggtgatggcagaaaagcgtggtgaaggacttgatcccaactcaccagaagctattgagcaggcgaagaagaaggcggaacgtaaggctcgtaatgagcgttcccgcaagattgccgcggagcgtaaagctaatgcggctcccgtgattgttccggagcgttctgatgtctccaccgatactccaaccgcggcaccaccgttctggggaacccgcattgtcaagggtctgcccttggcggagttcttgggcaaccttgatgagcgcgccttgttcatggggcagtggggtctgaaatccacccgcggcaacgagggtccaagctatgaggatttggtggaaactgaaggccgaccacgcctgcgctactggctggatcgcctgaagtctgagggcattttggaccacgtggccttggtgtatggctacttcccagcggtcgcggaaggcgatgacgtggtgatcttggaatccccggatccacacgcagccgaacgcatgcgctttagcttcccacgccagcagcgcggcaggttcttgtgcatcgcggatttcattcgcccacgcgagcaagctgtcaaggacggccaagtggacgtcatgccattccagctggtcaccatgggtaatcctattgctgatttcgccaacgagttgttcgcagccaatgaataccgcgagtacttggaagttcacggcatcggcgtgcagctcaccgaagcattggccgagtactggcactcccgagtgcgcagcgaactcaagctgaacgacggtggatctgtcgctgattttgatccagaagacaagaccaagttcttcgacctggattaccgcggcgcccgcttctcctttggttacggttcttgccctgatctggaagaccgcgcaaagctggtggaattgctcgagccaggccgtatcggcgtggagttgtccgaggaactccagctgcacccagagcagtccacagacgcgtttgtgctctaccacccagaggcaaagtactttaacgtctaacacctttgagagggaaaactttcccgc

SEQ ID NO: 2 - protein, 1221 aa, Corynebacterium glutamicum
  CDS (1)..(1221)
  Xaa(834)= Met or Ile
MSTSVTSPAHNNAHSSEFLDALANHVLIGDGAMGTQLQGFDLDVEKDFLDLEGCNEILNDTRPDVLRQIHRAYFEAGADLVETNTFGCNLPNLADYDIADRCRELAYKGTAVAREVADEMGPGRNGMRRFVVGSLGPGTKLPSLGHAPYADLRGHYKEAALGIIDGGGDAFLIETAQDLLQVKAAVHGVQDAMAELDTFLPIICHVTVETTGTMLMGSEIGAALTALQPLGIDMIGLNCATGPDEMSEHLRYLSKHADIPVSVMPNAGLPVLGKNGAEYPLEAEDLAQALAGFVSEYGLSMVGGCCGTTPEHIRAVRDAVVGVPEQETSTLTKIPAGPVEQASREVEKEDSVASLYTSVPLSQETGISMIGERTNSNGSKAFREAMLSGDWEKCVDIAKQQTRDGAHMLDLCVDYVGRDGTADMATLAALLATSSTLPIMIDSTEPEVIRTGLEHLGGRSIVNSVNFEDGDGPESRYQRIMKLVKQHGAAVVALTIDEEGQARTAEHKVRIAKRLIDDITGSYGLDIKDIVVDCLTFPISTGQEETRRDGIETIEAIRELKKLYPEIHTTLGLSNISFGLNPAARQVLNSVFLNECIEAGLDSAIAHSSKILPMNRIDDRQREVALDMVYDRRTEDYDPLQEFMQLFEGVSAADAKDARAEQLAAMPLFERLAQRIIDGDKNGLEDDLEAGMKEKSPIAIINEDLLNGMKTVGELFGSGQMQLPFVLQSAETMKTAVAYLEPFMEEEAEATGSAQAEGKGKIVVATVKGDVHDIGKNLVDIILSNNGYDVVNLGIKQPLSAMLEAAEEHKADVIGMSGLLVKSTVVMKENLEEXNNAGASNYPVILGGAALTRTYVENDLNEVYTGEVYYARDAFEGLRLMDEVMAEKRGEGLDPNSPEAIEQAKKKAERKARNERSRKIAAERKANAAPVIVPERSDVSTDTPTAAPPFWGTRIVKGLPLAEFLGNLDERALFMGQWGLKSTRGNEGPSYEDLVETEGRPRLRYWLDRLKSEGILDHVALVYGYFPAVAEGDDVVILESPDPHAAERMRFSFPRQQRGRFLCIADFIRPREQAVKDGQVDVMPFQLVTMGNPIADFANELFAANEYREYLEVHGIGVQLTEALAEYWHSRVRSELKLNDGGSVADFDPEDKTKFFDLDYRGARFSFGYGSCPDLEDRAKLVELLEPGRIGVELSEELQLHPEQSTDAFVLYHPEAKYFNV

SEQ ID NO: 3 - DNA, 1981 bp, Corynebacterium glutamicum
  CDS (101)..(1951)
tcaatattccgaagaaaaccgcgcagctctctcactagtctcaggtgaggcgaaagtggtgaaagacccgctacgcatggtgcgcctggctttttagaatgtgctgcaaacctcctggcatttctctatcctggcaggcatgactgatacctctccgttgaattctcagccgagtgcagatcaccaccctgatcacgcggctcgcccagttcttgatgcccacggcttgatcgttgagcacgaatcggaagagtttccagtccccgcacccgctcccggtgaacagccctgggagaagaaaaaccgcgagtggtacaaagacgccgttttctacgaagtgctggttcgtgccttctacgatccagaaggcaacggagtcggatcgttgaaaggcctgaccgaaaaactggattacatccagtggctcggcgtggattgcatttggatcccaccgttttatgattccccactgcgcgacggcggttacgatatccgcaacttccgtgaaatcctgcccgaattcggcaccgtcgatgacttcgtggaactcgttgaccacgcccaccgccgtggcctgcgtgttatcaccgacttggtcatgaatcacacctccgaccagcacgcatggttccaagaatcccggcgcgacccaaccggcccctacggagatttctatgtgtggagcgatgatcccaccctgtacaacgaagcccgcatcatctttgtagatacagaagaatccaactggacctatgatccggtgcgtggccagtacttctggcaccgcttcttctcccaccaaccagacctcaactacgacaaccccgcagtccaagaggccatgctagatgtcttgcgtttctggctggacctgggacttgatggtttccgactagatgccgttccttatctttttgaacgcgaaggcaccaacggcgaaaacctcaaagaaacccacgatttcctcaaactgtgtcgctctgtcattgagaaggaataccccggccgaatcctgctcgcagaagccaaccaatggccccaagatgtggtcgaatacttcggtgaaaaagacaaaggcgatgaatgccacatggccttccacttccctttgatgccgcgcatcttcatgggagttcgccaaggttcacgcaccccgatcagtgagatcctggccaacaccccggagattcccaagactgcccaatggggtattttcctgcgtaatcatgatgagctcacccttgaaatggtctccgatgaggaacgcagctacatgtactcccaattcgcctccgaacctcgcatgcgcgccaacgtaggaatccgcaggcgcctttccccactgcttgaaggcgaccgcaaccagctggaactccttcacggtttgttgctgtctctacctggctcacccgtgttgtattacggtgatgaaattggcatgggcgacaatatctggctccacgaccgcgacggagtgcgcacccccatgcagtggtccaacgaccgcaacggtggtttctccaaagctgatcctgaacgcctgtaccttccagcgatccaaaatgatcaatacggctacgcccaagtaaacgtggaaagccaactcaaccgcgaaaactccctgctgcgctggctccgaaaccaaatccttatccgcaagcagtaccgcgcatttggtgccggaacctaccgtgaagtgtcctccaccaatgagtcagtgttgacatttttacgagaacacaagggccaaaccattttgtgtgtcaacaacatgagcaaatatcctcaggcagtctcgcttgatttgcgtgaatttgcaggacacacccctcgagagatgtcgggcgggcagctgttccctaccattgctgaacgggagtggattgtcactttagcccctcacggattcttctggtttgatctcaccgccgatgaaaaggacgatatggaatgagcattggccaacacatcatcaccgagc

SEQ ID NO: 4 - protein, 617 aa, Corynebacterium glutamicum
VLQTSWHFSILAGMTDTSPLNSQPSADHHPDHAARPVLDAHGLIVEHESEEFPVPAPAPGEQPWEKKNREWYKDAVFYEVLVRAFYDPEGNGVGSLKGLTEKLDYIQWLGVDCIWIPPFYDSPLRDGGYDIRNFREILPEFGTVDDFVELVDHAHRRGLRVITDLVMNHTSDQHAWFQESRRDPTGPYGDFYVWSDDPTLYNEARIIFVDTEESNWTYDPVRGQYFWHRFFSHQPDLNYDNPAVQEAMLDVLRFWLDLGLDGFRLDAVPYLFEREGTNGENLKETHDFLKLCRSVIEKEYPGRILLAEANQWPQDVVEYFGEKDKGDECHMAFHFPLMPRIFMGVRQGSRTPISEILANTPEIPKTAQWGIFLRNHDELTLEMVSDEERSYMYSQFASEPRMRANVGIRRRLSPLLEGDRNQLELLHGLLLSLPGSPVLYYGDEIGMGDNIWLHDRDGVRTPMQWSNDRNGGFSKADPERLYLPAIQNDQYGYAQVNVESQLNRENSLLRWLRNQILIRKQYRAFGAGTYREVSSTNESVLTFLREHKGQTILCVNNMSKYPQAVSLDLREFAGHTPREMSGGQLFPTIAEREWIVTLAPHGFFWFDLTADEKDDME

SEQ ID NO: 5 - DNA, 1840 bp, Corynebacterium glutamicum
  CDS (363)..(1676)
cagaaactgtgtgcagaaatgcatgcagaaaaaggaaagttcgggccaagatgggtgtttctgtatgccgatgatcggatctttgacagctgggtatgcgacaaatcaccgagagttgttaattcttaacaatggaaaagtaacattgagagatgatttataccatcctgcaccatttagagtggggctagtcatacccccataaccctagctgtacgcaatcgatttcaaatcagttggaaaaagtcaagaaaattacccgagaattaatttataccacacagtctattgcaatagaccaagctgttcagtagggtgcatgggagaagaatttcctaataaaaactcttaaggacctccaaatgccaaagtacgacaattccaatgctgaccagtggggctttgaaacccgctccattcacgcaggccagtcagtagacgcacagaccagcgcacgaaaccttccgatctaccaatccaccgctttcgtgttcgactccgctgagcacgccaagcagcgtttcgcacttgaggatctaggccctgtttactcccgcctcaccaacccaaccgttgaggctttggaaaaccgcatcgcttccctcgaaggtggcgtccacgctgtagcgttctcctccggacaggccgcaaccaccaacgccattttgaacctggcaggagcgggcgaccacatcgtcacctccccacgcctctacggtggcaccgagactctattccttatcactcttaaccgcctgggtatcgatgtttccttcgtggaaaaccccgacgaccctgagtcctggcaggcagccgttcagccaaacaccaaagcattcttcggcgagactttcgccaacccacaggcagacgtcctggatattcctgcggtggctgaagttgcgcaccgcaacagcgttccactgatcatcgacaacaccatcgctaccgcagcgctcgtgcgcccgctcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctagctttaaatagactcaccccagtgcttaaagcgctgggtttttctttttcagactcgtgagaatgcaaactagactagacagagctgtccatatacactggacgaagttttagtcttgtccacccagaacaggcggttattttcatgcccaccctcgcgccttca

SEQ ID NO: 6 - protein, 437 aa, Corynebacterium glutamicum
MPKYDNSNADQWGFETRSIHAGQSVDAQTSARNLPIYQSTAFVFDSAEHAKQRFALEDLGPVYSRLTNPTVEALENRIASLEGGVHAVAFSSGQAATTNAILNLAGAGDHIVTSPRLYGGTETLFLITLNRLGIDVSFVENPDDPESWQAAVQPNTKAFFGETFANPQADVLDIPAVAEVAHRNSVPLIIDNTIATAALVRPLELGADVVVASLTKFYTGNGSGLGGVLIDGGKFDWTVEKDGKPVFPYFVTPDAAYHGLKYADLGAPAFGLKVRVGLLRDTGSTLSAFNAWAAVQGIDTLSLRLERHNENAIKVAEFLNNHEKVEKVNFAGLKDSPWYATKEKLGLKYTGSVLTFEIKGGKDEAWAFIDALKLHSNLANIGDVRSLVVHPATTTHSQSDEAGLARAGVTQSTVRLSVGIETIDDIIADLEGGFAAI

SEQ ID NO: 7 - DNA, 1033 bp, Corynebacterium glutamicum
  CDS (101)..(1006)
gtgcggatcgggtatccgcgctacacttagaggtgttagagatcatgagtttccacgaactgtaacgcaggattcaccaatcaatgaaaggtcgaccgacatgagcactgaagacattgtcgtcgtagcagtagatggctcggacgcctcaaaacaagctgttcggtgggctgcaaataccgccaacaaacgtggcattccacttcgcttggcttccagctacaccatgcctcagttcctctacgcagagggaatggttccaccacaagagcttttcgatgacctccaggccgaagccctggaaaagattaacgaagcccgtgacatcgcccatgaggtagcgccagaaatcaagatcgggcacaccatcgctgaaggcagtcccatcgacatgctgttggaaatgtctcccgatgccacaatgatcgtcatgggttcccgcggactcggcggactctccggaatggtcatgggctccgtctccggtgcagtggtcagccacgcaaagtgtccagtcgttgttgtccgtgaagacagcgcagtcaacgaagacagcaagtacggcccagtcgtcgtcggtgtggatggctccgaagtctcccaacaggcaaccgaatacgcatttgcggaagctgaagctcgtggcgccgaactcgttgcagttcacacctggatggacatgcaggtacaggcatcacttgcaggtcttgcagctgctcaacagcagtgggatgaagtggaacgtcagcaaaccgacatgctgatcgaacgcctcgcaccactggtggaaaagtacccaagtgtaaccgtcaagaagatcatcacccgtgaccgcccagttcgcgcacttgcagaagcatctgaaaacgcgcagctcctagtcgttggttcccatggtcgtggcggatttaagggcatgctccttggctccacctcccgcgcactgctgcaatccgcaccgtgcccaatgatggtggttcgcccacctgagaagattaagaagtagtttcttttaagtttcgatgccccggtt

SEQ ID NO: 8 - protein, 301 aa, Corynebacterium glutamicum
MSTEDIVVVAVDGSDASKQAVRWAANTANKRGIPLRLASSYTMPQFLYAEGMVPPQELFDDLQAEALEKINEARDIAHEVAPEIKIGHTIAEGSPIDMLLEMSPDATMIVMGSRGLGGLSGMVMGSVSGAVVSHAKCPVVVVREDSAVNEDSKYGPVVVGVDGSEVSQQATEYAFAEAEARGAELVAVHTWMDMQVQASLAGLAAAQQQWDEVERQQTDMLIERLAPLVEKYPSVTVKKIITRDRPVRALAEASENAQLLVVGSHGRGGFKGMLLGSTSRALLQSAPCPMMVVRPPEKIKK

SEQ ID NO: 9 - DNA, 1527 bp, Corynebacterium glutamicum
  CDS (101)..(1504): RXS00315
ctcatggcatctgcgccgttcgcgttcttgccagtgttggttggtttcaccgcaaccaagcgtttcggcggcaatgagttcctgggcgccgcgtattggtatggcgatggtgttcccgagcttggtgaacggctacgacgtggccgccaccatggctgcgggcgaaatgccaatgtggtccctgtttggtttagatgttgcccaagccggttaccagggcaccgtgcttcctgtgctggtggtttcttggattctggcaacgatcgagaagttcctgcacaagcgactcaagggcactgcagacttcctgatcactccagtgctgacgttgctgctcaccggattccttacattcatcgccattggcccagcaatgcgctgggtgggcgatgtgctggcacacggtctacagggactttatgatttcggtggtccagtcggcggtctgctcttcggtctggtctactcaccaatcgtcatcactggtctgcaccagtccttcccgccaattgagctggagctgtttaaccagggtggatccttcatcttcgcaacggcatctatggctaatatcgcccagggtgcggcatgtttggcagtgttcttcctggcgaagagtgaaaagctcaagggccttgcaggtgcttcaggtgtctccgctgttcttggtattacggagcctgcgatcttcggtgtgaaccttcgcctgcgctggccgttcttcatcggtatcggtaccgcagctatcggtggcgctttgattgcactctttaatatcaaggcagttgcgttgggcgctgcaggtttcttgggtgttgtttctattgatgctccagatatggtcatgttcttggtgtgtgcagttgttaccttcttcatcgcattcggcgcagcgattgcttatggcctttacttggttcgccgcaacggcagcattgatccagatgcaaccgctgctccagtgcctgcaggaacgaccaaagccgaagcagaagcacccgcagaattttcaaacgattccaccatcatccaggcacctttgaccggtgaagctattgcactgagcagcgtcagcgatgccatgtttgccagcggaaagcttggctcgggcgttgccatcgtcccaaccaaggggcagttagtttctccggtgagtggaaagattgtggtggcattcccatctggccatgctttcgcagttcgcaccaaggctgaggatggttccaatgtggatatcttgatgcacattggtttcgacacagtaaacctcaacggcacgcactttaacccgctgaagaagcagggcgatgaagtcaaagcaggggagctgctgtgtgaattcgatattgatgccattaaggctgcaggttatgaggtaaccacgccgattgttgtttcgaattacaagaaaaccggacctgtaaacacttacggtttgggcgaaattgaagcgggagccaacctgctcaacgtcgcaaagaaagaagcggtgccagcaacaccataagttgaaaccttgagtgttcg

SEQ ID NO: 10 - protein, 468 aa, Corynebacterium glutamicum
MAMVFPSLVNGYDVAATMAAGEMPMWSLFGLDVAQAGYQGTVLPVLVVSWILATIEKFLHKRLKGTADFLITPVLTLLLTGFLTFIAIGPAMRWVGDVLAHGLQGLYDFGGPVGGLLFGLVYSPIVITGLHQSFPPIELELFNQGGSFIFATASMANIAQGAACLAVFFLAKSEKLKGLAGASGVSAVLGITEPAIFGVNLRLRWPFFIGIGTAAIGGALIALFNIKAVALGAAGFLGVVSIDAPDMVMFLVCAVVTFFIAFGAAIAYGLYLVRRNGSIDPDATAAPVPAGTTKAEAEAPAEFSNDSTIIQAPLTGEAIALSSVSDAMFASGKLGSGVAIVPTKGQLVSPVSGKIVVAFPSGHAFAVRTKAEDGSNVDILMHIGFDTVNLNGTHFNPLKKQGDEVKAGELLCEFDIDAIKAAGYEVTTPIVVSNYKKTGPVNTYGLGEIEAGANLLNVAKKEAVPATP

SEQ ID NO: 11 - DNA, 2187 bp, Corynebacterium glutamicum
  CDS (101)..(2164): RXN01299
cgactgcggcgtctcttcctggcactaccattcctcgtcctgaccaactcgccacagctggtgcaacggtcacccaagtcaaaggattgaaagaatcagcatgaatagcgtaaataattcctcgcttgtccggctggatgtcgatttcggcgactccaccacggatgtcatcaacaaccttgccactgttattttcgacgctggccgagcttcctccgccgacgcccttgccaaagacgcgctggatcgtgaagcaaagtccggcaccggcgttcctggtcaagttgctatcccccactgccgttccgaagccgtatctgtccctaccttgggctttgctcgcctgagcaagggtgtggacttcagcggacctgatggcgatgccaacttggtgttcctcattgcagcacctgctggcggcggcaaagagcacctgaagatcctgtccaagcttgctcgctccttggtgaagaaggatttcatcaaggctctgcaggaagccaccaccgagcaggaaatcgtcgacgttgtcgatgccgtgctcaacccagcaccaaaaaccaccgagccagctgcagctccggctgcggcggcggttgctgagagtggggcggcgtcgacaagcgttactcgtatcgtggcaatcaccgcatgcccaaccggtatcgcacacacctacatggctgcggattccctgacgcaaaacgcggaaggccgcgatgatgtggaactcgttgtggagactcagggctcttccgctgtcaccccagtcgatccgaagatcatcgaagctgccgacgccgtcatcttcgccaccgacgtgggagttaaagaccgcgagcgtttcgctggcaagccagtcattgaatccggcgtcaagcgcgcgatcaatgagccagccaagatgatcgacgaggccatcgcagcctccaagaacccaaacgcccgcaaggtttccggttccggtgtcgcggcatctgctgaaaccaccggcgagaagctcggctggggcaagcgcatccagcaggcagtcatgaccggcgtgtcctacatggttccattcgtagctgccggcggcctcctgttggctctcggcttcgcattcggtggatacgacatggcgaacggctggcaagcaatcgccacccagttctctctgaccaacctgccaggcaacaccgtcgatgttgacggcgtggccatgaccttcgagcgttcaggcttcctgttgtacttcggcgcagtcctgttcgccaccggccaagcagccatgggcttcatcgtggcagccctgtctggctacaccgcatacgcacttgctggacgcccaggcatcgcgccgggcttcgtcggtggcgccatctccgtcaccatcggcgctggcttcattggtggtctggttaccggtatcttggctggtctcattgccctgtggattggctcctggaaggtgccacgcgtggtgcagtcactgatgcctgtggtcatcatcccgctacttacctcagtggttgttggtctcgtcatgtacctcctgctgggtcgcccactcgcatccatcatgactggtttgcaggactggctatcgtcaatgtccggaagctccgccatcttgctgggtatcatcttgggcctcatgatgtgtttcgacctcggcggaccagtaaacaaggcagcctacctctttggtaccgcaggcctgtctaccggcgaccaagcttccatggaaatcatggccgcgatcatggcagctggcatggtcccaccaatcgcgttgtccattgctaccctgctgcgcaagaagctgttcaccccagcagagcaagaaaacggcaagtcttcctggctgcttggcctggcattcgtctccgaaggtgccatcccattcgccgcagctgacccattccgtgtgatcccagcaatgatggctggcggtgcaaccactggtgcaatctccatggcactgggcgtcggctctcgggctccacacggcggtatcttcgtggtctgggcaatcgaaccatggtggggctggctcatcgcacttgcagcaggcaccatcgtgtccaccatcgttgtcatcgcactgaagcagttctggccaaacaaggccgtcgctgcagaagtcgcgaagcaagaagcacaacaagcagctgtaaacgcataatcggaccttgacccgatgtc

SEQ ID NO: 12 - protein, 688 aa, Corynebacterium glutamicum
MNSVNNSSLVRLDVDFGDSTTDVINNLATVIFDAGRASSADALAKDALDREAKSGTGVPGQVAIPHCRSEAVSVPTLGFARLSKGVDFSGPDGDANLVFLIAAPAGGGKEHLKILSKLARSLVKKDFIKALQEATTEQEIVDVVDAVLNPAPKTTEPAAAPAAAAVAESGAASTSVTRIVAITACPTGIAHTYMAADSLTQNAEGRDDVELVVETQGSSAVTPVDPKIIEAADAVIFATDVGVKDRERFAGKPVIESGVKRAINEPAKMIDEAIAASKNPNARKVSGSGVAASAETTGEKLGWGKRIQQAVMTGVSYMVPFVAAGGLLLALGFAFGGYDMANGWQAIATQFSLTNLPGNTVDVDGVAMTFERSGFLLYFGAVLFATGQAAMGFIVAALSGYTAYALAGRPGIAPGFVGGAISVTIGAGFIGGLVTGILAGLIALWIGSWKVPRVVQSLMPVVIIPLLTSVVVGLVMYLLLGRPLASIMTGLQDWLSSMSGSSAILLGIILGLMMCFDLGGPVNKAAYLFGTAGLSTGDQASMEIMAAIMAAGMVPPIALSIATLLRKKLFTPAEQENGKSSWLLGLAFVSEGAIPFAAADPFRVIPAMMAGGATTGAISMALGVGSRAPHGGIFVVWAIEPWWGWLIALAAGTIVSTIVVIALKQFWPNKAVAAEVAKQEAQQAAVNA

SEQ ID NO: 13 - DNA, 416 bp, Corynebacterium glutamicum
  CDS (1)..(393): RXA00951
atccaagcaatcttagagaaggcagcagcgccggcgaagcagaaggctcctgctgtggctcctgctgtaacacccactgacgctcctgcagcctcagtccaatccaaaacccacgacaagatcctcaccgtctgtggcaacggcttgggtacctccctcttcctcaaaaacacccttgagcaagttttcgacacctggggttggggtccatacatgacggtggaggcaaccgacactatctccgccaagggcaaagccaaggaagctgatctcatcatgacctctggtgaaatcgcccgcacgttgggtgatgttggaatcccggttcacgtgatcaatgacttcacgagcaccgatgaaatcgatgctgcgcttcgtgaacgctacgacatctaactactttaaaaggacgaaaa

SEQ ID NO: 14 - protein, 131 aa, Corynebacterium glutamicum
IQAILEKAAAPAKQKAPAVAPAVTPTDAPAASVQSKTHDKILTVCGNGLGTSLFLKNTLEQVFDTWGWGPYMTVEATDTISAKGKAKEADLIMTSGEIARTLGDVGIPVHVINDFTSTDEIDAALRERYDI

SEQ ID NO: 15 - DNA, 1827 bp, Corynebacterium glutamicum
  CDS (101)..(1804): RXN01244
gatatgtgtttgtttgtcaatatccaaatgtttgaatagttgcacaactgttggttttgtggtgatcttgaggaaattaactcaatgattgtgaggatgggtggctactgtggctgatgtgaatcaagacactgtactgaagggcaccggcgttgtcggtggagtccgttatgcaagcgcggtgtggattaccccacgccccgaactaccccaagcaggcgaagtcgtcgccgaagaaaaccgtgaagcagagcaggagcgtttcgacgccgctgcagccacagtctcttctcgtttgcttgagcgctccgaagctgctgaaggaccagcagctgaggtgcttaaagctactgctggcatggtcaatgaccgtggctggcgtaaggctgtcatcaagggtgtcaagggtggtcaccctgcggaatacgccgtggttgcagcaacaaccaagttcatctccatgttcgaagccgcaggcggcctgatcgcggagcgcaccacagacttgcgcgacatccgcgaccgcgtcatcgcagaacttcgtggcgatgaagagccaggtctgccagctgtttccggacaggtcattctctttgcagatgacctctccccagcagacaccgcggcactagacacagatctctttgtgggacttgtcactgagctgggtggcccaacgagccacaccgcgatcatcgcacgccagctcaacgtgccttgcatcgtcgcatccggcgccggcatcaaggacatcaagtccggcgaaaaggtgcttatcgacggcagcctcggcaccattgaccgcaacgcggacgaagctgaagcaaccaagctcgtctccgagtccctcgagcgcgctgctcgcatcgccgagtggaagggtcctgcacaaaccaaggacggctaccgcgttcagctgttggccaacgtccaagacggcaactctgcacagcaggctgcacagaccgaagcagaaggcatcggcctgttccgcaccgaactgtgcttcctttccgccaccgaagagccaagcgttgatgagcaggctgcggtctactcaaaggtgcttgaagcattcccagagtccaaggtcgttgtccgctccctcgacgcaggttctgacaagccagttccattcgcatcgatggctgatgagatgaacccagcactgggtgttcgtggcctgcgtatcgcacgtggacaggttgatctgctgactcgccagctcgacgcaattgcgaaggccagcgaagaactcggccgtggcgacgacgccccaacctgggttatggctccaatggtggctaccgcttatgaagcaaagtggtttgctgacatgtgccgtgagcgtggcctaatcgccggcgccatgatcgaagttccagcagcatccctgatggcagacaagatcatgcctcacctggactttgtttccatcggtaccaacgacctgacccagtacaccatggcagcggaccgcatgtctcctgagcttgcctacctgaccgatccttggcagccagcagtcctgcgcctgatcaagcacacctgtgacgaaggtgctcgctttaacaccccggtcggtgtttgtggtgaagcagcagcagacccactgttggcaactgtcctcaccggtcttggcgtgaactccctgtccgcagcatccactgctctcgcagcagtcggtgcaaagctgtcagaggtcaccctggaaacctgtaagaaggcagcagaagcagcacttgacgctgaaggtgcaactgaagcacgcgatgctgtacgcgcagtgatcgacgcagcagtctaaaccactgttgagctaaaaag

SEQ ID NO: 16 - protein, 568 aa, Corynebacterium glutamicum
VATVADVNQDTVLKGTGVVGGVRYASAVWITPRPELPQAGEVVAEENREAEQERFDAAAATVSSRLLERSEAAEGPAAEVLKATAGMVNDRGWRKAVIKGVKGGHPAEYAVVAATTKFISMFEAAGGLIAERTTDLRDIRDRVIAELRGDEEPGLPAVSGQVILFADDLSPADTAALDTDLFVGLVTELGGPTSHTAIIARQLNVPCIVASGAGIKDIKSGEKVLIDGSLGTIDRNADEAEATKLVSESLERAARIAEWKGPAQTKDGYRVQLLANVQDGNSAQQAAQTEAEGIGLFRTELCFLSATEEPSVDEQAAVYSKVLEAFPESKVVVRSLDAGSDKPVPFASMADEMNPALGVRGLRIARGQVDLLTRQLDAIAKASEELGRGDDAPTWVMAPMVATAYEAKWFADMCRERGLIAGAMIEVPAASLMADKIMPHLDFVSIGTNDLTQYTMAADRMSPELAYLTDPWQPAVLRLIKHTCDEGARFNTPVGVCGEAAADPLLATVLTGLGVNSLSAASTALAAVGAKLSEVTLETCKKAAEAALDAEGATEARDAVRAVIDAAV

SEQ ID NO: 17 - DNA, 390 bp, Corynebacterium glutamicum
  CDS (101)..(367): RXA01300
gatcgacattaaatcccctcccttggggggtttaactaacaaatcgctgcgccctaatccgttcggattaacggcgtagcaacacgaaaggacactttccatggcttccaagactgtaaccgtcggttcctccgttggcctgcacgcacgtccagcatccatcatcgctgaagcggctgctgagtacgacgacgaaatcttgctgaccctggttggctccgatgatgacgaagagaccgacgcgtcctcttccctcatgatcatggcgctgggcgcagagcacggcaacgaagttaccgtcacctccgacaacgctgaagctgttgagaagatcgctgcgcttatcgcacaggaccttgacgctgagtaaacaacgctctgcttgttaaa

SEQ ID NO: 18 - protein, 89 aa, Corynebacterium glutamicum
MASKTVTVGSSVGLHARPASIIAEAAAEYDDEILLTLVGSDDDEETDASSSLMIMALGAEHGNEVTVTSDNAEAVEKIAALIAQDLDAE

SEQ ID NO: 19 - DNA, 508 bp, Corynebacterium glutamicum
  CDS (101)..(508): RXN03002
ggaacttcgaggtgtcttcgtggggcgtacggagatctagcaagtgtggctttatgtttgaccctatccgaatcaacatgcagtgaattaacatctacttatgtttgtactcaaagatctgctaaaggcagaacgcatagaactcgaccgcacggtcaccgattggcgtgaaggcatccgcgccgcaggtgtactcctagaaaagacaaacagcattgattccgcctacaccgatgccatgatcgccagcgtggaagaaaaaggcccctacattgtggtcgctccaggtttcgctttcgcgcacgcccgccccagcagagcagtccgcgagaccgctatgtcgtgggtgcgcctggcctcccctgtttccttcggtcacagtaagaatgatcccctcaatctcatcgttgctctcgctgccaaagatgccaccgcacatacccaagcgatggcggcattggctaaagctttaggaaaataccgaaaggatctcgacgaggcacaaagt

SEQ ID NO: 20 - protein, 136 aa, Corynebacterium glutamicum
MFVLKDLLKAERIELDRTVTDWREGIRAAGVLLEKTNSIDSAYTDAMIASVEEKGPYIVVAPGFAFAHARPSRAVRETAMSWVRLASPVSFGHSKNDPLNLIVALAAKDATAHTQAMAALAKALGKYRKDLDEAQS

SEQ ID NO: 21 - DNA, 789 bp, Corynebacterium glutamicum
  CDS (14)..(766)
cttgcattccccaatggcgccaccaacggtaggcaactacatcatgcagtccttcactcaaggtctgcagttcggcgttgcagttgccgtgattctctttggtgtccgcaccattcttggtgaactggtccccgcattccaaggtattgctgcgaaggttgttcccggagctatccccgcattggatgcaccgatcgtgttcccctacgcgcagaacgccgttctcattggtttcttgtcttccttcgtcggtggcttggttggcctgactgttcttgcatcgtggctgaacccagcttttggtgtcgcgttgattctgcctggtttggtcccccacttcttcactggtggcgcggcgggcgtttacggtaatgccacgggtggtcgtcgaggagcagtatttggcgcctttgccaacggtcttctgattaccttcctccctgctttcctgcttggtgtgcttggttccttcgggtcagagaacaccactttcggtgatgcggactttggttggttcggaatcgttgttggttctgcagccaaggtggaaggtgctggcgggctcatcttgttgctcatcatcgcagcggttcttctgggtggcgcgatggtcttccagaagcgcgtcgtgaatgggcactgggatccagctcccaaccgtgagcgcgtggagaaggcggaagctgatgccactccaacggctggggctcggacctaccctaagattgctcctccggcgggcgctcctaccccaccggctcgaagctaagatctccaaaaccctgagat

SEQ ID NO: 22 - protein, 251 aa, Corynebacterium glutamicum
MAPPTVGNYIMQSFTQGLQFGVAVAVILFGVRTILGELVPAFQGIAAKVVPGAIPALDAPIVFPYAQNAVLIGFLSSFVGGLVGLTVLASWLNPAFGVALILPGLVPHFFTGGAAGVYGNATGGRRGAVFGAFANGLLITFLPAFLLGVLGSFGSENTTFGDADFGWFGIVVGSAAKVEGAGGLILLLIIAAVLLGGAMVFQKRVVNGHWDPAPNRERVEKAEADATPTAGARTYPKIAPPAGAPTPPARS

SEQ ID NO: 23 - DNA, 553 bp, Corynebacterium glutamicum
  CDS (101)..(553): RXC03001
cccggttcacgtgatcaatgacttcacgagcaccgatgaaatcgatgctgcgcttcgtgaacgctacgacatctaactactttaaaaggacgaaaatattatggactggttaaccattcctcttttcctcgttaatgaaatccttgcggttccggctttcctcatcggtatcatcaccgccgtgggattgggtgccatggggcgttccgtcggtcaggttatcggtggagcaatcaaagcaacgttgggctttttgctcattggtgcgggtgccacgttggtcactgcctccctggagccactgggtgcgatgatcatgggtgccacaggcatgcgtggtgttgtcccaacgaatgaagccatcgccggaatcgcacaggctgaatacggcgcgcaggtggcgtggctgatgattctgggcttcgccatctctttggtgttggctcgtttcaccaacctgcgttatgtcttgctcaacggacaccacgtgctgttgatgtgcaccatgctcaccatggtcttggccaccggaagagttgatgcgtggatcttc

SEQ ID NO: 24 - protein, 151 aa, Corynebacterium glutamicum
MDWLTIPLFLVNEILAVPAFLIGIITAVGLGAMGRSVGQVIGGAIKATLGFLLIGAGATLVTASLEPLGAMIMGATGMRGVVPTNEAIAGIAQAEYGAQVAWLMILGFAISLVLARFTNLRYVLLNGHHVLLMCTMLTMVLATGRVDAWIF

SEQ ID NO: 25 - DNA, 2172 bp, Corynebacterium glutamicum
  CDS (101)..(2149): RXN01943
ccgattctttttcggcccaattcgtaacggcgatcctcttaagtggacaagaaagtctcttgcccgcgggagacagaccctacgtttagaaaggtttgacatggcgtccaaactgacgacgacatcgcaacatattctggaaaaccttggtggaccagacaatattacttcgatgactcactgtgcgactcgccttcgcttccaagtgaaggatcaatccattgttgatcaacaagaaattgactccgacccatcagttcttggcgtagtaccccaaggatccaccggtatgcaggtggtgatgggtggatctgttgcaaactattaccaagaaatcctcaaacttgatggaatgaagcacttcgccgacggtgaagctacagagagttcatccaagaaggaatacggcggagtccgtggcaagtactcgtggattgactacgccttcgagttcttgtctgatactttccgaccaatcctgtgggccctgcttggtgcctcactgattattaccttgttggttcttgcggatactttcggtttgcaagacttccgcgctccaatggatgagcagcctgatacttatgtattcctgcactccatgtggcgctcggtcttctacttcctgccaattatggttggtgccaccgcagctcgaaagctcggcgcaaacgagtggattggtgcagctattccagccgcacttcttactccagaattcttggcactgggttctgccggcgataccgtcacagtctttggcctgccaatggttctgaatgactactccggacaggtattcccaccgctgattgcagcaattggtctgtactgggtggaaaagggactgaagaagatcatccctgaagcagtccaaatggtgttcgtcccattcttctccctgctgattatgatcccagcgaccgcattcctgcttggacctttcggcatcggtgttggtaacggaatttccaacctgcttgaagcgattaacaacttcagcccatttattctttccatcgttatcccattgctctacccattcttggttccacttggattgcactggccactaaacgccatcatgatccagaacatcaacaccctgggttacgacttcattcagggaccaatgggtgcctggaacttcgcctgcttcggcctggtcaccggcgtgttcttgctctccattaaggaacgaaacaaggccatgcgtcaggtttccctgggtggcatgttggctggtttgctcggcggcatttccgagccttccctctacggtgttctgctccgattcaagaagacctacttccgcctcctgccgggttgtttggcaggcggtatcgtgatgggcatcttcgacatcaaggcgtacgctttcgtgttcacctccttgcttaccatcccagcaatggacccatggttgggctacaccattggtatcgcagttgcattcttcgtttccatgttccttgttctcgcactggactaccgttccaacgaagagcgcgatgaggcacgtgcaaaggttgctgctgacaagcaggcagaagaagatctgaaggcagaagctaatgcaactcctgcagctccagtagctgctgcaggtgcgggagccggtgcaggtgcaggagccgctgctggcgctgcaaccgccgtggcagctaagccgaagctggccgctggggaagtagtggacattgtttccccactcgaaggcaaggcaattccactttctgaagtacctgacccaatctttgcagcaggcaagcttggaccaggcattgcaatccaaccaactggaaacaccgttgttgctccagcagacgctactgtcatccttgtccagaaatctggacacgcagtggcattgcgcttagatagcggagttgaaatccttgtccacgttggattggacaccgtgcaattgggcggcgaaggcttcaccgttcacgttgagcgcaggcagcaagtcaaggcgggggatccactgatcacttttgacgctgacttcattcgatccaaggatctacctttgatcaccccagttgtggtgtctaacgccgcgaaattcggtgaaattgaaggtattcctgcagatcaggcaaattcttccacgactgtgatcaaggtcaacggcaagaacgagtaacctgggatccatgttgcgca

SEQ ID NO: 26 - protein, 683 aa, Corynebacterium glutamicum
MASKLTTTSQHILENLGGPDNITSMTHCATRLRFQVKDQSIVDQQEIDSDPSVLGVVPQGSTGMQVVMGGSVANYYQEILKLDGMKHFADGEATESSSKKEYGGVRGKYSWIDYAFEFLSDTFRPILWALLGASLIITLLVLADTFGLQDFRAPMDEQPDTYVFLHSMWRSVFYFLPIMVGATAARKLGANEWIGAAIPAALLTPEFLALGSAGDTVTVFGLPMVLNDYSGQVFPPLIAAIGLYWVEKGLKKIIPEAVQMVFVPFFSLLIMIPATAFLLGPFGIGVGNGISNLLEAINNFSPFILSIVIPLLYPFLVPLGLHWPLNAIMIQNINTLGYDFIQGPMGAWNFACFGLVTGVFLLSIKERNKAMRQVSLGGMLAGLLGGISEPSLYGVLLRFKKTYFRLLPGCLAGGIVMGIFDIKAYAFVFTSLLTIPAMDPWLGYTIGIAVAFFVSMFLVLALDYRSNEERDEARAKVAADKQAEEDLKAEANATPAAPVAAAGAGAGAGAGAAAGAATAVAAKPKLAAGEVVDIVSPLEGKAIPLSEVPDPIFAAGKLGPGIAIQPTGNTVVAPADATVILVQKSGHAVALRLDSGVEILVHVGLDTVQLGGEGFTVHVERRQQVKAGDPLITFDADFIRSKDLPLITPVVVSNAAKFGEIEGIPADQANSSTTVIKVNGKNE

SEQ ID NO: 27 - DNA, 372 bp, Corynebacterium glutamicum
  CDS (101)..(349): RXA01503
gtatcctcaaaggccttctagctgttgcagctgcagcgcactcggtggatacgacatccacgacctatcaaattctttatgctgcaggcgatgccttttcatgttcttggcagtcattttggcgattactgcggctcgtaaattcggtgccaatgtctttacatcagtcgcactcgctggtgcattgctgcacacacagcttcaggcagtaaccgtgttggttgacggtgaactccagtcgatgactctggtggctttccaaaaggctggtaatgacgtcaccttcctgggcattccagtggtgctgcagttggcgttgcatgtagcgagtttgatgaagttgtcgcgataagaggaggggcgtgtcggtct

SEQ ID NO: 28 - protein, 83 aa, Corynebacterium glutamicum
MFLAVILAITAARKFGANVFTSVALAGALLHTQLQAVTVLVDGELQSMTLVAFQKAGNDVTFLGIPVVLQLALHVASLMKLSR

SEQ ID NO: 29 - DNA, 1578 bp, Corynebacterium glutamicum
  CDS (101)..(1555): RXN00351
gaaggctgctgctaagaaaacgaccaagaagaccactaagaaaactactaaaaagaccaccgcaaagaagaccacaaagaagtcttaagccggatcttatatggatgattccaatagctttgtagttgttgctaaccgtctgccagtggatatgactgtccacccagatggtagctatagcatctcccccagccccggtggccttgtcacggggctttcccccgttctggaacaacatcgtggatgttgggtcggatggcctggaactgtagatgttgcacccgaaccatttcgaacagatacgggtgttttgctgcaccctgttgtcctcactgcaagtgactatgaaggcttctacgagggcttttcaaacgcaacgctgtggcctcttttccacgatctgattgttactccggtgtacaacaccgattggtggcatgcgtttcgggaggtaaacctcaagttcgctgaagccgtgagccaagtggcggcacacggtgccactgtgtgggtgcaggactatcagctgttgctggttcctggcattttgcgccagatgcgccctgatttgaagatcggtttcttcctccacattcccttcccttcccctgatctgttccgtcagctgccgtggcgtgaagagattgttcgaggcatgctgggcgcagatttggtgggattccatttggttcaaaacgcagaaaacttccttgcgttaacccagcaggttgccggcactgccgggtctcatgtgggtcagccggacaccttgcaggtcagtggtgaagcattggtgcgtgagattggcgctcatgttgaaaccgctgacggaaggcgagttagcgtcggggcgttcccgatctcgattgatgttgaaatgtttggggaggcgtcgaaaagcgccgttcttgatcttttaaaaacgctcgacgagccggaaaccgtattcctgggcgttgaccgactggactacaccaagggcattttgcagcgcctgcttgcgtttgaggaactgctggaatccggcgcgttggaggccgacaaagctgtgttgctgcaggtcgcgacgccttcgcgtgagcgcattgatcactatcgtgtgtcgcgttcgcaggtcgaggaagccgtcggccgtatcaatggtcgtttcggtcgcatggggcgtcccgtggtgcattatctacacaggtcattgagcaaaaatgatctccaggtgctgtataccgcagccgatgtcatgctggttacgccttttaaagacggtatgaacttggtggctaaagaattcgtggccaaccaccgcgacggcactggtgctttggtgctgtccgaatttgccggcgcggccactgagctgaccggtgcgtatttatgcaacccatttgatgtggaatccatcaaacggcaaatggtggcagctgtccatgatttgaagcacaatccggaatctgcggcaacgcgaatgaaaacgaacagcgagcaggtctatacccacgacgtcaacgtgtgggctaatagtttcctggattgtttggcacagtcgggagaaaactcatgaaccgcgcacgaatcgcgacc

SEQ ID NO: 30 - protein, 485 aa, Corynebacterium glutamicum
MDDSNSFVVVANRLPVDMTVHPDGSYSISPSPGGLVTGLSPVLEQHRGCWVGWPGTVDVAPEPFRTDTGVLLHPVVLTASDYEGFYEGFSNATLWPLFHDLIVTPVYNTDWWHAFREVNLKFAEAVSQVAAHGATVWVQDYQLLLVPGILRQMRPDLKIGFFLHIPFPSPDLFRQLPWREEIVRGMLGADLVGFHLVQNAENFLALTQQVAGTAGSHVGQPDTLQVSGEALVREIGAHVETADGRRVSVGAFPISIDVEMFGEASKSAVLDLLKTLDEPETVFLGVDRLDYTKGILQRLLAFEELLESGALEADKAVLLQVATPSRERIDHYRVSRSQVEEAVGRINGRFGRMGRPVVHYLHRSLSKNDLQVLYTAADVMLVTPFKDGMNLVAKEFVANHRDGTGALVLSEFAGAATELTGAYLCNPFDVESIKRQMVAAVHDLKHNPESAATRMKTNSEQVYTHDVNVWANSFLDCLAQSGENS

SEQ ID NO: 31 - DNA, 891 bp, Corynebacterium glutamicum
  CDS (101)..(868): RXA00347
tcggccagcaatccgcttggtgtcctggatcgcgccgacatcttaaggtgccagggctttaaagtgccaggggttctgtgggatccgtacactggttcccatgactttgactattgaggaaatcgccaagaccaaaaagcttttggttgtgtccgattttgatggaaccatcgcaggatttagcaaggacgcttacaacgttcctatcaaccagaaatccctcaaggcggtaaaagacctctcccaacaagcagacactgatgttgtcattttgtcgggacgtcacctggagggattgaagacggttcttgatcttggtcagtacgacatcaccatggtgggttcacacggttctgaggattcctcccgcccgcgtaccctcactcctgaagaggtagctcgcctcgccaagattgaagcagatctggaaaagatcgtcgacggcatcgaaggcgcattcgtggagatcaagcctttccaccgcgtgctgcacttcatccgtgtttccgacaaggacaaagtccaaggaatcctcgcccaagcagcacacgtagactcttccggcctgaaggttactaacggcaagagcatcatcgaatactccatcagctccaccaccaagggcacctggctgaaggaatacgttgaccgcaccgagcccactggtgtgattttcctcggcgatgacaccaccgatgagcacggtttcaaagctttagaaaacgatgatcgtgccctaaccgtcaaggttggcgaaggagacactgcagccaaaacccgcgtcgacgatgttgataatgtgggaattttcctagagaaactcgcctaccaccgcatgcagtatgcggaaagcgtgcgattggggatttaagagagcctaaacgcacgaaa

SEQ ID NO: 32 - protein, 256 aa, Corynebacterium glutamicum
MTLTIEEIAKTKKLLVVSDFDGTIAGFSKDAYNVPINQKSLKAVKDLSQQADTDVVILSGRHLEGLKTVLDLGQYDITMVGSHGSEDSSRPRTLTPEEVARLAKIEADLEKIVDGIEGAFVEIKPFHRVLHFIRVSDKDKVQGILAQAAHVDSSGLKVTNGKSIIEYSISSTTKGTWLKEYVDRTEPTGVIFLGDDTTDEHGFKALENDDRALTVKVGEGDTAAKTRVDDVDNVGIFLEKLAYHRMQYAESVRLGI

SEQ ID NO: 33 - DNA, 2556 bp, Corynebacterium glutamicum
  CDS (101)..(2533): RXN01239
gcacttgctgcgtaaatctttttcccacgccgggaatgcgtgaacactaagatcgaggacgtaccgcacgattttgcctaacttttaagggtgtttcatcatggcacgtccaatttccgcaacgtacaggcttcaaatgcgaggacctcaagcagatagcgccgggcgttcatttggttttgcgcaggccaaagcccagcttccctatctgaagaagctaggcatcagccacctgtacctctcccctatttttacggccatgccagattccaatcatggctacgatgtcattgatcccaccaccatcaatgaagagctcggtggcatggagggtcttcgagatcttgccgcagctacacacgagttgggcatgggcatcatcattgatattgttcccaaccatttaggtgttgccgttccacatttgaatccttggtggtgggatgttctaaaaaacggcaaagattccgcttttgagttctatttcgatattgactggcacgaagacaacggttctggtggcaagctgggcatgccgattctgggtgctgaaggcgatgaagacaagctggaattcgcggagcttgatggagagaaagtgctcaaatattttgaccacctcttcccaatcgcgcctggtaccgaagaagggacaccgcaagaagtctacaagcgccagcattaccgcctgcagttctggcgcgatggcgtgatcaacttccgtcgcttcttttccgtgaatacgttggctggcatcaggcaagaagatcccttagtgtttgaacatactcatcgtctgctgcgcgaattggtggcggaagacctcattgacggcgtgcgcgtcgatcaccccgacgggctttccgatccttttggatatctgcacagactccgcgacctcattggacctgaccgctggctgatcatcgaaaagatcttgagcgttgatgaaccactcgatccccgcctggccgttgatggcaccactggctacgacgccctccgtgaactcgacggcgtgtttatctcccgagaatctgaggacaaattctccatgctggcgctgacccacagtggatccacctgggatgaacgcgccctcaaatccacggaggaaagcctcaaacgagtcgtcgcccaacaagaactcgcagccgaaatcttaaggctcgcccgcgccatgcgccgcgataacttctccaccgcaggcaccaacgtcaccgaagacaaacttagcgaaaccatcatcgaattagtcgccgccatgcccgtctaccgcgccgactacatctccctctcacgcaccaccgccaccgtcatcgcggagatgtccaaacgcttcccctcccggcgtgacgcactcgacctcatcgcggccgccctacttggcaatggcgaggccaaaatccgcttcgctcaagtctgcggcgccgtcatggctaaaggtgtggaagacaccaccttctaccgcgcatctaggctcgttgcattgcaagaagtcggtggcgcgccggggagattcggcgtctccgctgcagaattccacttgctgcaggaagaacgcagcctgctgtggccacgcaccatgaccaccttgtccacgcatgacaccaaacgtggcgaagatacccgcgcccgcatcatctccctgtctgaagtccccgatatgtactccgagctggtcaatcgtgttttcgcggtgctccccgcgccagacggcgcaacgggcagtttcctcctacaaaacctgctgggcgtatggcccgccgacggcgtgatcaccgatgcgctgcgcgatcgattcagggaatacgccctaaaagctatccgcgaagcatccacaaaaaccacgtgggtggaccccaacgagtccttcgaggctgcggtctgcgattgggtggaagcgcttttcgacggaccctccacctcactaatcaccgaatttgtctcccacatcaaccgtggctctgtgcaaatctccttaggcaggaaactgctgcaaatggtgggcgctggaatccccgacacttaccaaggaactgagtttttagaagactccctggtagatcccgataaccgacgctttgttgattacaccgccagagaacaagtcctggagcgcctgcaaacctgggcttggacgcaggttaattcggtagaagacttggtggataacgccgacatcgccaaaatggccgtggtccataaatccctcgagttgcgtgctgaatttcgtgcaagctttgttggtggagatcatcaggcagtatttggcgaaggtcgcgcagaatcccacatcatgggcatcgcccgcggtacagaccgaaaccacctcaacatcattgctcttgctacccgtcgaccactgatcttggaagaccgtggcggatggtatgacaccaccgtcacgcttcctggtggacaatgggaagacaggctcaccgggcaacgcttcagtggtgttgtcccagccaccgatttgttctcacatctacccgtatctttgttggttttagtacccgatagtgagttttgatccctgcacaggaaagttag

SEQ ID NO: 34 - protein, 811 aa, Corynebacterium glutamicum
MARPISATYRLQMRGPQADSAGRSFGFAQAKAQLPYLKKLGISHLYLSPIFTAMPDSNHGYDVIDPTTINEELGGMEGLRDLAAATHELGMGIIIDIVPNHLGVAVPHLNPWWWDVLKNGKDSAFEFYFDIDWHEDNGSGGKLGMPILGAEGDEDKLEFAELDGEKVLKYFDHLFPIAPGTEEGTPQEVYKRQHYRLQFWRDGVINFRRFFSVNTLAGIRQEDPLVFEHTHRLLRELVAEDLIDGVRVDHPDGLSDPFGYLHRLRDLIGPDRWLIIEKILSVDEPLDPRLAVDGTTGYDALRELDGVFISRESEDKFSMLALTHSGSTWDERALKSTEESLKRVVAQQELAAEILRLARAMRRDNFSTAGTNVTEDKLSETIIELVAAMPVYRADYISLSRTTATVIAEMSKRFPSRRDALDLIAAALLGNGEAKIRFAQVCGAVMAKGVEDTTFYRASRLVALQEVGGAPGRFGVSAAEFHLLQEERSLLWPRTMTTLSTHDTKRGEDTRARIISLSEVPDMYSELVNRVFAVLPAPDGATGSFLLQNLLGVWPADGVITDALRDRFREYALKAIREASTKTTWVDPNESFEAAVCDWVEALFDGPSTSLITEFVSHINRGSVQISLGRKLLQMVGAGIPDTYQGTEFLEDSLVDPDNRRFVDYTAREQVLERLQTWAWTQVNSVEDLVDNADIAKMAVVHKSLELRAEFRASFVGGDHQAVFGEGRAESHIMGIARGTDRNHLNIIALATRRPLILEDRGGWYDTTVTLPGGQWEDRLTGQRFSGVVPATDLFSHLPVSLLVLVPDSEF

SEQ ID NO: 35 - DNA, 1953 bp, Corynebacterium glutamicum
  CDS (101)..(1930): RXA02645
gatacagctccttgatggagtgaataaattcgcgagcctgctcctgatcttgcacacgcgtgatataggtcagaaatcgcgagcgcttgatctctagttcatgctcaaagacttgaccggcctgagggagttggtattgcgtgagatgtgccatagcatctcacatcttagctcgccaaccggcagcattttcactagcctggtggccatgttgacctcgcaaagcttttcagtgtgggctccacttccccacgatgtacatctgatcctcaacggcgaaaccctccccatgcacaaaacggagggcagctggtggcgcgccgagatcgcgcccaaggccggcgatcgttacggtttttcgcttttcgacggctcctcctggtcaaaaaccctccccgatccccgctccacatctcaaccagacggggttcatggtttaagtgaagtctccgatgattcctatctgtggggtgaccagcagtggactggccgaattctccctggctcggtgttatatgagctgcatgtgggcacctttagtgaagatggaacgtttgagggagtcgtcgacaagcttccttatctgcgcgacctcggcgtgaccgccatcgaacttttacccgtgcagccctttggcggcaaccgcaattggggctacgacggggtgctgtggcacgccgtccatgcaggctacggcggtccggcgggcttgaaaaagcttatcgacgcctcccaccaggccggcatcgccgtctacttagacgtcgtgtacaaccacttcggccccgacggcaactacaacgggcaatttggcccctacacctctggcggcagcaccggctggggcgacgtggtcaacatcaacggccatgattcagatgaagtccgcaattatattctcgacgccgcacgccagtggttcgaagattttcacgttgatgggctccgcctcgatgcggtgcattctctcgatgatcgcggcgcctattccctacttgcgcagctgaccatggtggccgaggatgtctccgcacaaacaggcatcccacgctcattgattgcagaatctgaactcaatgaccccaagttcgttacctcccgcgaggccggcggttttggcctggatgcacagtgggttgacgatatccaccacgccctccatgccctcgtttctggcgaacgcaatggttattacagcgatttcggatctgtcgacacattagccaaaaccctgcgtgaagtatttgaacacaccggaaactactccacgtaccgcggacgcaaccacggccgccctgtgcaccccgatatcacccctgcctcgcgctttgtcacctacaccaccacccatgatcagaccggcaaccgcgcaatcggcgaccgtccttccacgactctcaccccggaacagcaggtgttgaaggcagccattatctacagctcgccgtataccccgatgttgtttatgggtgaagaattcggagccaccaccccattcgccttcttttgctcccacaccgaccccgagctcaaccggctaacctccgagggccgcaaacgggaattcgcacgccttggctggaacgccgacgacatcccctcccccgagctggaatccaccttcacctcctccaaactcgattgggagttcactgcggagcagcgccgcatcaacgacgcttacaagcagctgttgcacctgcggcacaccttgggcttctcccaaccaaacttgctcacactcgaggttgagcacggcgagaactggctatcgatggccaatggtcgcggccgaattctggcgaatttctccgacgacaccatcaccgtcccgcttggcggcgagctgatttacagcttcacttcccccaccgtcaccgacacctccacaacccttcagccgtggggctttgcgatcctgacccgaaactagaaaaaggccacctcgattga

SEQ ID NO: 36 - protein, 610 aa, Corynebacterium glutamicum
MLKDLTGLRELVLREMCHSISHLSSPTGSIFTSLVAMLTSQSFSVWAPLPHDVHLILNGETLPMHKTEGSWWRAEIAPKAGDRYGFSLFDGSSWSKTLPDPRSTSQPDGVHGLSEVSDDSYLWGDQQWTGRILPGSVLYELHVGTFSEDGTFEGVVDKLPYLRDLGVTAIELLPVQPFGGNRNWGYDGVLWHAVHAGYGGPAGLKKLIDASHQAGIAVYLDVVYNHFGPDGNYNGQFGPYTSGGSTGWGDVVNINGHDSDEVRNYILDAARQWFEDFHVDGLRLDAVHSLDDRGAYSLLAQLTMVAEDVSAQTGIPRSLIAESELNDPKFVTSREAGGFGLDAQWVDDIHHALHALVSGERNGYYSDFGSVDTLAKTLREVFEHTGNYSTYRGRNHGRPVHPDITPASRFVTYTTTHDQTGNRAIGDRPSTTLTPEQQVLKAAIIYSSPYTPMLFMGEEFGATTPFAFFCSHTDPELNRLTSEGRKREFARLGWNADDIPSPELESTFTSSKLDWEFTAEQRRINDAYKQLLHLRHTLGFSQPNLLTLEVEHGENWLSMANGRGRILANFSDDTITVPLGGELIYSFTSPTVTDTSTTLQPWGFAILTRN

SEQ ID NO: 37 - DNA, 832 bp, Corynebacterium glutamicum
  CDS (101)..(832): RXN02355
atttttgaccctccgggggtgatttaacctaaaattccacacaaacgtgttcgaggtcattagattgataagcatctgttgttaagaaaggtgacttcctatgtcctcgatttcccgcaagaccggcgcgtcacttgcagccaccacactgttggcagcgatcgcactggccggttgtagttcagactcaagctccgactccacagattccaccgctagcgaaggcgcagacagccgcggccccatcacctttgcgatgggcaaaaacgacaccgacaaagtcattccgatcatcgaccgctggaacgaagcccaccccgatgagcaggtaacgctcaacgaactcgccggtgaagccgacgcgcagcgcgaaaccctcgtgcaatccctgcaggccggcaactctgactacgacgtcatggcgctcgacgtcatctggaccgcagacttcgcggcaaaccaatggctcgcaccacttgaaggcgacctcgaggtagacacctccggactgctgcaatccaccgtggattccgcaacctacaacggcaccctctacgcactgccacagaacaccaacggccagctactgttccgcaacaccgaaatcatcccagaagcaccagcaaactgggctgacctcgtggaatcctgcacgcttgctgaagaagcaggcgttgattgcctgaccactcagctcaagcagtacgaaggcctttcagtgaacaccatcggcttcatcgaaggttggggaggcagcgtcctagacgatgacggcaaacgtcaccgtagacagcacgacggcaaggcaggccttcaagcgcttgtcgacggc

SEQ ID NO: 38 - protein, 244 aa, Corynebacterium glutamicum
MSSISRKTGASLAATTLLAAIALAGCSSDSSSDSTDSTASEGADSRGPITFAMGKNDTDKVIPIIDRWNEAHPDEQVTLNELAGEADAQRETLVQSLQAGNSDYDVMALDVIWTADFAANQWLAPLEGDLEVDTSGLLQSTVDSATYNGTLYALPQNTNGQLLFRNTEIIPEAPANWADLVESCTLAEEAGVDCLTTQLKQYEGLSVNTIGFIEGWGGSVLDDDGKRHRRQHDGKAGLQALVDG

SEQ ID NO: 39 - DNA, 609 bp, Corynebacterium glutamicum
  CDS (101)..(586): RXN02909
caacgcgaatgaaaacgaacagcgagcaggtctatacccacgacgtcaacgtgtgggctaatagtttcctggattgtttggcacagtcgggagaaaactcatgaaccgcgcacgaatcgcgaccataggcgttcttccgcttgctttactgctggcgtcctgtggttcagacaccgtggaaatgacagattccacctggttggtgaccaatatttacaccgatccagatgagtcgaattcgatcagtaatcttgtcatttcccagcccagcttagattttggcaattcttccctgtctggtttcactggctgtgtgccttttacggggcgtgcggaattcttccaaaatggtgagcaaagctctgttctggatgccgattatgtgaccttgtcttccctggatttcgataaacttcccgatgattgccaaggacaagaactcaaagttcataacgagctggttgatcttctgcctggttcttttgaaatctccaggacttctggttcagaaatcttgctgactagcgatgtcgatgaactcgatcggccagcaatccgcttggtgtcctggatcgcgccgacatcttaaggtgccagggctttaaagtg

SEQ ID NO: 40 - protein, 162 aa, Corynebacterium glutamicum
MNRARIATIGVLPLALLLASCGSDTVEMTDSTWLVTNIYTDPDESNSISNLVISQPSLDFGNSSLSGFTGCVPFTGRAEFFQNGEQSSVLDADYVTLSSLDFDKLPDDCQGQELKVHNELVDLLPGSFEISRTSGSEILLTSDVDELDRPAIRLVSWIAPTS

SEQ ID NO: 41 - DNA, 1590 bp, Corynebacterium glutamicum
  CDS (101)..(1567): RXS00349
tgtgtacatcacaatggaattcggggctagagtatctggtgaaccgtgcataaacgacctgtgattggactctttttccttgcaaaatgttttccagcggatgttgagttttgcgacccttcgtggccgcatttcaacagttgacgctgcaaaagccgcacctccgccatcgccactagccccgattgatctcactgaccatagtcaagtggccggtgtgatgaatttggctgcgagaattggcgatattttgctttcttcaggtacgtcaaatagtgacaccaaggtacaagttcgagcagtgacctctgcgtacggtttgtactacacgcacgtggatatcacgttgaatacgatcaccatcttcaccaacatcggtgtggagaggaagatgccggtcaacgtgtttcatgttgtaggcaagttggacaccaacttctccaaactgtctgaggttgaccgtttgatccgttccattcaggctggtgcgaccccgcctgaggttgccgagaaaatcctggacgagttggagcaatcccctgcgtcttatggtttccctgttgcgttgcttggctgggcaatgatgggtggtgctgttgctgtgctgttgggtggtggatggcaggtttccctaattgcttttattaccgcgttcacgatcattgccacgacgtcatttttgggaaagaagggtttgcctactttcttccaaaatgttgttggtggttttattgccacgctgcctgcatcgattgcttattctttggcgttgcaatttggtcttgagatcaaaccgagccagatcatcgcatctggaattgttgtgctgttggcaggtttgacactcgtgcaatctctgcaggacggcatcacgggcgctccggtgacagcaagtgcacgatttttcgaaacactcctgtttaccggcggcattgttgctggcgtgggtttgggcattcagctttctgaaatcttgcatgtcatgttgcctgccatggagtccgctgcagcacctaattattcgtctacattcgcccgcattatcgctggtggcgtcaccgcagcggccttcgcagtgggttgttacgcggagtggtcctcggtgattattgcggggcttactgcgctgatgggttctgcgttttattacctcttcgttgtttatttaggccccgtctctgccgctgcgattgctgcaacagcagttggtttcactggtggtttgcttgcccgtcgattcttgattccaccgttgattgtggcgattgccggcatcacaccaatgcttccaggtctagcaatttaccgcggaatgtacgccaccctgaatgatcaaacactcatgggtttcaccaacattgcggttgctttagccactgcttcatcacttgccgctggcgtggttttgggtgagtggattgcccgcaggctacgtcgtccaccacgcttcaacccataccgtgcatttaccaaggcgaatgagttctccttccaggaggaagctgagcagaatcagcgccggcagagaaaacgtccaaagactaatcagagattcggtaataaaaggtaaaaatcaacctgcttaggcgt

SEQ ID NO: 42 - protein, 489 aa, Corynebacterium glutamicum
MLSFATLRGRISTVDAAKAAPPPSPLAPIDLTDHSQVAGVMNLAARIGDILLSSGTSNSDTKVQVRAVTSAYGLYYTHVDITLNTITIFTNIGVERKMPVNVFHVVGKLDTNFSKLSEVDRLIRSIQAGATPPEVAEKILDELEQSPASYGFPVALLGWAMMGGAVAVLLGGGWQVSLIAFITAFTIIATTSFLGKKGLPTFFQNVVGGFIATLPASIAYSLALQFGLEIKPSQIIASGIVVLLAGLTLVQSLQDGITGAPVTASARFFETLLFTGGIVAGVGLGIQLSEILHVMLPAMESAAAPNYSSTFARIIAGGVTAAAFAVGCYAEWSSVIIAGLTALMGSAFYYLFVVYLGPVSAAAIAATAVGFTGGLLARRFLIPPLIVAIAGITPMLPGLAIYRGMYATLNDQTLMGFTNIAVALATASSLAAGVVLGEWIARRLRRPPRFNPYRAFTKANEFSFQEEAEQNQRRQRKRPKTNQRFGNKR

SEQ ID NO: 43 - DNA, 440 bp, Corynebacterium glutamicum
  CDS (1)..(417): RXS03183
gaagccgaagcaaccgcaggcaaattcgaagtacagcccctcgtaggtaaagacggcgtcggcgtatccacccttggtggctacaacaacggcatcaacgtcaactccgaaaacaaggcaaccgcccgcgacttcatcgaattcatcatcaacgaagagaaccaaacctggttcgcggacaactccttcccaccagttctggcatccatctacgatgatgagtcccttgttgagcagtacccatacctgccagcactgaaggaatccctggaaaacgcagcaccacgcccagtgtctcctttctacccagccatctccaaggcaatccaggacaacgcctacgcagcgcttaacggcaacgtcgacgttgaccaggcaaccaccgatatgaaggcagcgatcgaaaacgcttccagctagttcggtaatttagttcattc

SEQ ID NO: 44 - protein, 139 aa, Corynebacterium glutamicum
EAEATAGKFEVQPLVGKDGVGVSTLGGYNNGINVNSENKATARDFIEFIINEENQTWFADNSFPPVLASIYDDESLVEQYPYLPALKESLENAAPRPVSPFYPAISKAIQDNAYAALNGNVDVDQATTDMKAAIENASS

SEQ ID NO: 45 - DNA, 1212 bp, Corynebacterium glutamicum
  CDS (101)..(1189): RXC00874
agctgttccctaccattgctgaacgggagtggattgtcactttagcccctcacggattcttctggtttgatctcaccgccgatgaaaaggacgatatggaatgagcattggccaacacatcatcaccgagcgtttctacggcgccaagtcccacaccatcgacaacgtagatattgtgttgtcccgcgaatgtggcgagaacactttggctgtagtgcgcatcaacaatgcgctgtatcagttgttggtcaatgatgatggcaaagatgttctcaacgaccacgtagaagaggtcggtgcgagtttcggagcatggactggcagctctgcttttcccattggccctttcactccactcggcacagaacaatccaatagctctttcatcaccgccgacaataaagcgatcgtgaaatacttccgcaaattagaatccgggcaaaaccccgatgtggagctaatttctaaaatttcctcctgccccaacatcgcgcccatcctgggtttttcctccgctgagatctccggggctaactacaccctggtcatggcgcagcagtacgttccaggtttggatggctggtcacacgcgctgactactacctctggcagctttgcagaggatgcagaaaagatcggcgaagccacccgcaatgttcacactgctcttgcatcggccttccctactcgggtagttcccgtagaagcactcgccgatgcgctcactacccgccttaatgaactaatctcccaagcacccgaaatcgcccgcttcaaagaagcagccatcgacctctaccaatcgttggaaggcgaagcccacatccaacgcatccacggtgacctccacttggggcagctcatcaaaacccccgaacgctacatcctcatcgatttcgaaggcgaacctgcccgcccacttaatcaacgacgcctccccgactctcccctgaaagatctcgccggcatcatcagatccatcgactacgcagcctacttcgacggcgaacacacccaatgggccaacgaagccaccgcgctattcctcgacggctacggatcaattgaagaccaagaactcctcaatgcctacattctggacaaggcgttgtacgaggttgcctatgaaataaacaaccgccccgactgggtgaaaatcccactcgaggcggtcgaaaggcttctagactagttagttactctgcgtcaaac

SEQ ID NO: 46 - protein, 363 aa, Corynebacterium glutamicum
MSIGQHIITERFYGAKSHTIDNVDIVLSRECGENTLAVVRINNALYQLLVNDDGKDVLNDHVEEVGASFGAWTGSSAFPIGPFTPLGTEQSNSSFITADNKAIVKYFRKLESGQNPDVELISKISSCPNIAPILGFSSAEISGANYTLVMAQQYVPGLDGWSHALTTTSGSFAEDAEKIGEATRNVHTALASAFPTRVVPVEALADALTTRLNELISQAPEIARFKEAAIDLYQSLEGEAHIQRIHGDLHLGQLIKTPERYILIDFEGEPARPLNQRRLPDSPLKDLAGIIRSIDYAAYFDGEHTQWANEATALFLDGYGSIEDQELLNAYILDKALYEVAYEINNRPDWVKIPLEAVERLLD